(19) United States
(12) Patent Application Publication
(10) Pub. No.: US 2026/0355492 A1
(43) Pub. Date: Nov. 12, 2026

(54) SYSTEMS AND METHODS FOR AUTOMATION IN DIALYSIS UNDER STERILE CONDITIONS

(71) Applicant: JRC Medical, Inc., Dover, DE (US)

(72) Inventors: Jie Shi YAN, Moorpark, CA (US); Richard WU, Thousand Oaks, CA (US); Cody CARGILL, Tujunga, CA (US)

(73) Assignee: JRC Medical, Inc., Dover, DE (US)

(21) Appl. No.: 19/672,443

(22) Filed: May 8, 2026

Related U.S. Application Data
(60) Provisional application No. 63/803,349, filed on May 9, 2025.

(51) Int. Cl. A61M 39/18; A61M 1/14; A61M 1/28

(52) U.S. Cl. CPC A61M 39/18; A61M 1/154; A61M 1/159; A61M 1/282

(57) ABSTRACT

Provided herein are exemplary systems and methods for automatically connecting various dialysis line connector types of conventional, commercially available dialysis lines together under sterile conditions to complete a dialysis circuit. The automated dialysis line connection system may comprise a series of cases, each configured to receive and automatically connect a pair of line connectors to thereby fluidically couple the corresponding dialysis lines. The appropriate case may be selected based on the line connector types of the dialysis lines to be connected. The system further may include one or more cases for automated clamping of unused dialysis lines, as well as for automated sampling of effluent fluid for monitoring one or more solute concentration levels therein. Additionally, a compact system for automatically storing and dispensing dialysate bags for use during a dialysis treatment is provided.

### CROSS-REFERENCE TO RELATED APPLICATIONS

This application claims priority to U.S. Provisional Patent Application Ser. No. 63/803,349, filed May 9, 2025, the entire contents of which are incorporated herein by reference.

### TECHNICAL FIELD

This technology generally relates to automated systems for connecting dialysis tubing lines, dialysate bags, the drain bag, and the peritoneal dialysis catheter under sterile conditions, and methods of use thereof.

### BACKGROUND

Peritoneal dialysis (PD) is a type of dialysis that uses the peritoneum in a person's abdomen as the membrane through which fluid and dissolved substances are exchanged with the blood. During peritoneal dialysis, a sterile dialysis solution (dialysate) is instilled into the peritoneal cavity and later drained through a peritoneal dialysis catheter. A typical peritoneal dialysis treatment requires 4-5 cycles of fluid exchange. Patients on PD perform their own dialysis at home. The PD can be done manually, called Continuous Ambulatory Peritoneal Dialysis (CAPD), or using a cycler, called Automated Peritoneal Dialysis (APD) or Continuous Cycling Peritoneal Dialysis (CCPD). APD or CCPD uses a cycler to automatically perform the exchange. Dialysis patients that perform automated peritoneal dialysis (APD or CCPD) have to make manual connections between the peritoneal dialysis catheter and the cycler, dialysate bags and the cycler, and the drain bag and the cycler. Patients also need to manually clamp unused tubing lines. These connections must all be performed under sterile conditions. If not handled carefully, infections can result.

There are interface systems to facilitate sterile connections. The transfer set at the external end of the PD catheter outside the patient's abdomen, and the cassette on the cycler are such interface systems. The transfer set is a short, sterile, flexible tubing component that connects the PD catheter to the cycler. The proximal end of the flexible tubing attaches securely to the external end of the PD catheter via a locking mechanism. The distal end of the flexible tubing connects to a line from the cassette, often with a built-in manual slide or twist clamp to control fluid flow. The cassette on a cycler is a single use, disposable, sterile, plastic cartridge that serves as the central fluid management component of the cycler. The cassette interfaces with the cycler to control the flow of dialysis solution during automated exchanges, and contains multiple lines (e.g., a patient line, solution lines, and a drain line) and a membrane chamber. The patient line connects to the transfer set on the PD catheter. The solution line connects to the dialysate bag. The drain line connects to the drain bag. The membrane chamber is a soft, flexible chamber that the cycler compresses or releases to pump fluid.

The end of the administration port of the dialysate bag, the transfer set, and the lines of the cassette are all sealed with pull rings or caps that need to be removed during connecting. The distal end of the transfer set of the PD catheter is sealed with a protective cap that needs to be removed during connecting. The connection between the PD catheter and the cycler is achieved by connecting the transfer set with the patient line of the cassette on the cycler. The connection between the dialysate bag and the cycler is achieved by connecting the administration port of the dialysate bag with the solution line of the cassette. The connection between the drain bag and the cycler is achieved by connecting the drain bag and the drain line of the cassette. After the dialysate bag is connected to the solution line of the cassette, a frangible seal, e.g., a small and rigid plastic breakable barrier built into the administration port of the dialysate bag, needs to be broken to allow fluid flow. Additional challenges include the manual handling of multiple dialysate bags and the lack of real-time monitoring of effluent chemistry during therapy.

In view of the foregoing drawbacks of previously known systems and methods, there exists a need for an automated PD connector system that connects the PD catheter, dialysate bags, the drain bag, and lines of the cassette under sterile conditions to reduce the chances of infection.

### SUMMARY

Provided herein are improved systems and methods for automatically connecting dialysis tubing lines, dialysate bags, the drain bag, and the PD catheter under sterile conditions, increasing safety compliance, and decreasing risk of infection. In accordance with one aspect, a system for automatically connecting a pair of dialysis lines of a dialysis circuit is provided. The system may include a fixed platform comprising a first slot configured to receive a first connector of a first dialysis line, a movable platform configured to move in at least two degrees of freedom relative to the fixed platform, a drive gear coupled to the fixed platform or the movable platform at an incline angle relative to the first slot or the second slot, and a controller operatively coupled to one or more motors of the movable platform. The movable platform may comprise a second slot configured to receive a second connector of a second dialysis line, and the drive gear may be configured to be actuated to transmit rotary motion and linear motion to the first connector or the second connector. The controller may be configured to: cause the one or more motors to move the movable platform in a first direction along a first degree of freedom to coaxially align the second connector with the first connector; cause the one or more motors to move the movable platform in a first direction along a second degree of freedom to engage the second connector with the first connector; and actuate the drive gear to transmit rotary motion and linear motion to the first connector or the second connector to cause the first connector or the second connector to be coupled to the second connector or the first connector, respectively. The system further may comprise a first cover configured to be removably disposed over the first connector within the first slot, and a second cover configured to be removably disposed over the second connector within the second slot.

The system further may comprise a first slide table comprising a first stationary frame and first slide block configured to move relative to the first stationary frame along the first or second degree of freedom, the first slide block coupled to the movable platform, and a second slide table comprising a second stationary frame and second slide block configured to move relative to the second stationary frame along the second or first degree of freedom, the second slide block coupled to the first stationary frame. The second stationary frame may be fixed relative to the fixed platform. Moreover, each of the first and second slide tables may comprise a threaded axel extending longitudinally between first and second ends of the respective first and second stationary frames, the threaded axel operatively coupled the one or more motors of the movable platform, and one or more support rods extending longitudinally between the first and second ends of the respective first and second stationary frames. The first and second slide blocks may each comprise a threaded channel configured to slidably receive the respective threaded axel therethrough, such that rotation of the respective threaded axel via the one or more motors of the movable platform may cause the respective first and second slide blocks to slide along the respective threaded axel and one or more support rods. For example, the first slide table may be offset from the second slide table by an angle of 90 degrees.

The first connector may comprise a plurality of ridges, a lip, and a removable cap comprising a pull ring. The first slot may comprise a cavity configured to receive the lip in a manner that prevents axial movement between the first connector and the first slot, a plurality of grooves may be configured to receive at least a portion of the plurality of ridges in a manner that prevents rotation between the first connector and the first slot, and a channel may be configured to be aligned with a tubing of the first dialysis line when the first connector is received within the first slot. Moreover, the movable platform may comprise a hook configured to be wrapped by the pull ring of the removable cap of the first connector. Accordingly, the system further may comprise a clamp block operatively coupled to a clamp motor, the clamp motor configured to be actuated via the controller to move the clamp block through the channel between a first disengaged position where the clamp block is disengaged from the tubing of the first dialysis line, and a second engaged position where the clamp block engages the tubing of the first dialysis line to thereby prevent fluid flow between the first connector and the tubing of the first dialysis line.

The first connector may comprise a twistable lock, an indented retention portion, and a removable cap comprising one or more gripping portions. Accordingly, the system further may comprise a retention gear configured to be disposed over the indented retention portion in a manner that permits transmission of rotary motion from the retention gear to the first connector. The first slot may comprise a cavity configured to slidably receive the retainer gear therein, an opening may be disposed within the cavity, and a channel may be configured to be aligned with the twistable lock when the first connector is received within the first slot. Moreover, the movable platform may comprise a cap holder configured to releasably receive the one or more gripping portions of the removable cap of the first connector. Accordingly, the system further may comprise a lock block operatively coupled to a lock motor, the lock motor configured to be actuated via the controller to move the lock block through the channel between a first disengaged position where the lock block is disengaged from the twistable lock, and a second engaged position where the lock block engages the twistable lock to thereby inhibit movement between the twistable lock and the first slot. Additionally, the drive gear may be coupled to the fixed platform, such that the drive gear extends at least partially through the opening to contact the retainer gear when the first connector is received within the first slot to thereby transmit rotary motion to the first connector.

The second connector may comprise a flat retention portion, a lip, and a removable cap comprising a pull ring. Accordingly, the system further may comprise a retention gear configured to be disposed over the flat retention portion in a manner that permits transmission of rotary motion and linear motion from the retention gear to the second connector. The second slot may comprise a cavity configured to slidably receive the retainer gear therein, an opening may be disposed within the cavity, and a channel may be configured to be aligned with a tubing of the second dialysis line when the second connector is received within the second slot. Moreover, the fixed platform may comprise a hook configured to be wrapped by the pull ring of the removable cap of the second connector. Accordingly, the system further may comprise a clamp block operatively coupled to a clamp motor, the clamp motor configured to be actuated via the controller to move the clamp block through the channel between a first disengaged position where the clamp block is disengaged from the tubing of the second dialysis line, and a second engaged position where the clamp block engages the tubing of the second dialysis line to thereby prevent fluid flow between the second connector and the tubing of the second dialysis line. In addition, the drive gear may be coupled to the movable platform at the incline angle relative to the second slot, such that the drive gear extends at least partially through the opening to contact the retainer gear when the second connector is received within the second slot to thereby transmit rotary motion and linear motion to the second connector.

In some embodiments, the second connector may comprise a plurality of ridges, a lip, and a removable cap comprising a pull ring, and the second slot may comprise a cavity comprising plurality of grooves configured to receive at least a portion of the plurality of ridges in a manner that prevents rotation between the second connector and the second slot, and a channel configured to be aligned with a tubing of the second dialysis line when the second connector is received within the second slot. Moreover, the fixed platform may comprise a hook configured to be wrapped by the pull ring of the removable cap of the second connector. Accordingly, the system further may comprise a clamp block operatively coupled to a clamp motor, the clamp motor configured to be actuated via the controller to move the clamp block through the channel between a first disengaged position where the clamp block is disengaged from the tubing of the second dialysis line, and a second engaged position where the clamp block engages the tubing of the second dialysis line to thereby prevent fluid flow between the second connector and the tubing of the second dialysis line. The movable platform further may comprise a tube holder configured to releasably receive at least a portion of the tubing of the second dialysis line therein.

In accordance with another aspect of the present disclosure, an automated dialysis system is provided. The automated dialysis system may comprise a first case, a second case, and a third case, each case comprising the system for automatically connecting a pair of dialysis lines of a dialysis circuit described above. For example, the first dialysis line of the first case may comprise a bag line fluidically coupled to a dialysis bag comprising dialysate, the second dialysis line of the first case may comprise a cycler solution line operatively coupled to a cycler, the first dialysis line of the second case may comprise a transfer set configured to be fluidically coupled to a patient's body, the second dialysis line of the second case may comprise a cycler patient line operatively coupled to the cycler, the first dialysis line of the third case may comprise a drain bag line fluidically coupled to a drain bag, and the second dialysis line of the third case may comprise a second cycler drain line operatively coupled to the cycler.

Accordingly, the controller may be configured to: cause the first case to fluidically couple the bag line with the cycler solution line and cause the third case to fluidically couple the drain bag line with the cycler drain line; cause the cycler to complete a priming process; and cause, upon completion of the priming process, the second case to fluidically couple the transfer set with the cycler solution line. The controller may be configured to receive a signal from the cycler indicative of the completion of the priming process. In some embodiments, the system further may comprise a sensor configured to measure a fluid level within the cycler patient line, such that the controller may be configured to determine that the priming process is complete based on the fluid level within the cycler patient line. Additionally, or alternatively, the system may comprise a sensor operatively coupled to the third case and configured to detect a velocity of fluid flow through the drain bag line. Accordingly, the controller may be configured to cause the cycler to cease draining of fluid from the patient's body when the velocity of fluid flow through the drain bag line falls below a predetermined threshold.

The controller may further be configured to: calculate an amount of fluid drained from the patient's body at the end of a first infusion cycle based at least partially on the velocity of fluid flow through the drain bag line; estimate an amount of fluid remaining within the patient's body at the end of the first infusion cycle based on the amount of fluid drained from the patient's body at the end of a first infusion cycle; determine an adjusted amount of fluid to be infused into the patient's body via the transfer set based on the estimated amount of fluid remaining within the patient's body at the end of the first infusion cycle; and cause, during a subsequent infusion cycle, the cycler to infuse the adjusted amount of fluid into the patient's body via the transfer set to thereby avoid overfilling the patient's body. In some embodiments, the system further may comprise a sensor configured to measure at least one of a glucose, blood urea nitrogen (BUN), or creatinine concentration of fluid exiting the patient's body, such that the controller may be configured to cause the cycler to initiate drainage of fluid from the patient's body via the drain bag line when at least one of the glucose, BUN, or creatinine concentration of the fluid exiting the patient's body is within a predetermined range.

The system further may comprise an automated clamping system for clamping one or more unused dialysis lines. For example, the automated clamping system may comprise a third slot comprising a cavity configured to receive at least a portion of a third connector of a third dialysis line, and a channel configured to be aligned with a tubing of the third dialysis line when the third connector is received within the third slot. In addition, the automated clamping system may comprise a clamp block operatively coupled to a clamp motor, the clamp motor configured to be actuated via the controller to move the clamp block through the channel between a first disengaged position where the clamp block is disengaged from the tubing of the third dialysis line, and a second engaged position where the clamp block engages the tubing of the third dialysis line to thereby prevent fluid flow between the third connector and the tubing of the third dialysis line.

In accordance with another aspect of the present disclosure, a system for automatically sampling effluent fluid of a dialysis circuit is provided. The system may comprise at least one test strip holder configured to receive a set of test strips therein, the set of test strips configured to generate electrical signals indicative of at least one solute concentration level of effluent fluid, an interposer configured to be coupled to the at least one test strip holder, the interposer comprising a plurality of test strip contacts configured to electrically contact the set of test strips within the at least one test strip holder, and a fluid collection chamber comprising at least one sampling well. The at least one sampling well may comprise a collection zone disposed therein, a first inlet in fluid communication with the collection zone and sized and shaped to receive effluent fluid therethrough, and a second inlet in fluid communication with the collection zone and sized and shaped to receive the at least one test strip holder therethrough. Additionally, the system may comprise an effluent tube having an inlet configured to be fluidically coupled to a drain line of the dialysis circuit, an outlet configured to be fluidically coupled to a drain bag of the dialysis circuit or a drain line extension tube, and a sampling port configured to be aligned with and fluidically sealed against the first inlet of the at least one sampling well.

The system further may comprise a sensor configured to generate data indicative of the at least one solute concentration level of the effluent fluid based on the electrical signals received from the plurality of test strip contacts, and at least one motor operatively coupled to the fluid collection chamber and the at least one test strip holder. Further, the system may comprise a controller operatively coupled to the sensor and the at least one motor, the controller configured to: cause the at least one motor to move the fluid collection chamber to a first position relative to the effluent tube where the first inlet of the at least one sampling well is aligned with the sampling port of the effluent tube to thereby permit effluent fluid to flow from the effluent tube into the collection zone; cause the at least one motor to move the at least one test strip holder through the second inlet of the at least one sampling well to a second position, such that the set of test strips are at least partially submerged in the effluent fluid within the collection zone; and generate information indicative of the at least one solute concentration level of the effluent fluid based on the data generated by the sensor.

For example, the set of test strips may comprise at least one of a glucose test strip, creatinine test strip, or a blood urea nitrogen (BUN) test strip. The at least one test strip holder may comprise a dedicated, insulated receptacle for each test strip of the set of test strips. Moreover, the plurality of test strip contacts of the interposer may comprise a plurality of spring fingers configured to press against the set of test strips within the at least one test strip holder to maintain electrical contact therebetween. In some embodiments, the at least one test strip holder may include four test strip holders equally spaced apart, and the fluid collection chamber may comprise four sampling wells. In addition, the interposer may comprise a contact pad in electrical communication with the plurality of test strip contacts, the contact pad configured to be electrically coupled to the sensor to transmit the electrical signals received from the plurality of test strip contacts thereto. Moreover, the sensor may comprise an array of pogo-pins configured to be in electrical contact with the contact pad to facilitate transmission of electrical signals therebetween. The array of pogo-pins may be spring-loaded to thereby facilitate consistent electrical contact between the array of pogo-pins and the contact pad. Further, the controller may be configured to cause the at least one motor to move the at least one test holder and the interposer relative to the fluid collection chamber to a third position, such that the contact pad is in electrical contact with the array of pogo-pins.

The interposer may comprise a L-shaped profile configured to facilitate coupling of the interposer to the at least one test strip holder. Additionally, the collection zone may be sized and shaped to receive a predetermined maximum volume of effluent fluid therein. Moreover, the at least one sampling well further may comprise a waste reservoir in fluid communication with the collection zone via an overflow channel, the overflow channel configured to permit effluent fluid in excess of the predetermined maximum volume within the collection zone to flow therethrough into the waste reservoir. For example, the collection zone may be at least partially defined by a vertical extending overflow weir, the overflow weir defining an inlet of the overflow channel. The fluid collection chamber may comprise a cylindrical profile, such that the effluent tube may comprise a curved portion configured to wrap at least partially around a sidewall of the fluid collection chamber. Accordingly, the sampling port may be disposed on the curved portion of the fluid collection chamber. In addition, the controller may be configured to cause the at least one motor to rotate the fluid collection chamber relative to the effluent tube to the first position.

Moreover, the fluid collection chamber, the interposer, and the at least one test strip holder may be coaxially locked together, such that the fluid collection chamber, the interposer, and the at least one test strip holder are configured to synchronously rotate together upon actuation of the at least one motor. Further, the sampling port may comprise a rectangular profile having a long axis that extends parallel to a longitudinal axis of the effluent tube. The at least one motor may be operatively coupled to the at least one test strip holder via a central column of the at least one test strip holder, the central column extending at least partially through a central channel of the fluid collection chamber. Additionally, the controller may be configured to maintain the fluid collection chamber in the first position for a predetermined time period. The predetermined time period may be selected to permit at least a predetermined volume of effluent fluid to flow from the effluent tube into the collection zone. In addition, the controller may be configured to maintain the at least one test strip holder in the second position for at least a predetermined dwell time period. The predetermined dwell time period may be selected to permit the set of test strips to complete a chemical reaction with the effluent fluid.

The controller further may be configured to cause the at least one motor to move the fluid collection chamber to a third position relative to the effluent tube where the sampling port of the effluent tube is not aligned with the first inlet of the at least one sampling well, such that effluent fluid is not permitted to flow from the effluent tube into the collection zone of the at least one sampling well. In addition, the system further may include a case configured to house the at least one test strip holder, the interposer, the fluid collection chamber, the effluent tube, and the sensor therein. The case may include a lid. Accordingly, the sensor may be disposed on an underside of the lid within the case. Moreover, the system may comprise a sliding lock configured to be transitioned between an unlocked state where the sliding lock is disengaged from the fluid collection chamber, and a locked state where the sliding lock applies a force against the fluid collection chamber to thereby facilitate sealing of the effluent tube against the fluid collection chamber. For example, the sliding lock may comprise a curved profile corresponding to a curvature of a sidewall of the fluid collection chamber. Additionally, the sliding lock may be attached to a spring configured to apply a spring force to facilitate sealing of the effluent tube against fluid collection chamber in the locked state. Further, the spring may be attached to a crossbar extending between longitudinally extending portions of the effluent tube adjacent to the inlet and the outlet of the effluent tube.

In accordance with another aspect, a system for automated loading and dispensing of dialysate bags for a dialysis treatment is provided. The system may comprise at least one mobile column configured to receive a plurality of dialysate bags therein, the at least one mobile column comprising a loading shelf operatively coupled to a first motor and sized and shaped to hold the plurality of dialysate bags thereon, and the loading shelf configured to be linearly movable between a first vertical position at a lower portion of the at least one mobile column and a second vertical position at an upper portion of the at least one mobile column via the first motor. In addition, the system may comprise a transfer module disposed above the at least one mobile column, the transfer module comprising an access window and a dispensing platform operatively coupled to a second motor. The dispensing platform may be configured to be linearly movable between a first loading position at a first end of the transfer module and a second dispensing position at a second end of the transfer module opposite the first end via the second motor. Moreover, the system may comprise a controller operatively coupled to the first and second motors, the controller configured to: cause the first motor to move the loading shelf to a third vertical position between the first and second vertical positions, such that, in the third vertical position, a top-most dialysate bag of the plurality of dialysate bags disposed on the loading shelf is horizontally aligned with the dispensing platform; and cause, when the top-most dialysate bag is linearly aligned with the access window, the second motor to move the dispensing platform from the first loading position to the second dispensing position, to thereby move the top-most dialysate bag through the access window.

At least one mobile column may be configured to be removed from underneath the transfer module to facilitate loading of the plurality of dialysate bags therein. For example, the at least one mobile column may comprise a plurality of wheels configured to provide mobility to the at least one mobile column. In some embodiments, the at least one mobile column may comprise three mobile columns. Moreover, the at least one mobile column may comprise a controller configured to receive user input indicative of at least one of dialysate concentrations of the plurality of dialysate bags loaded within the at least one mobile column or a quantity of dialysate bags loaded within the at least one mobile column. In addition, the at least one mobile column may comprise a sensor, e.g., an infrared sensor, configured to detect a presence of a dialysate bag within a top-most slot within the at least one mobile column.

The first motor may be operatively coupled to the loading shelf via a nut mounted on the loading shelf, the nut threadably and slidably coupled to a ball screw shaft extending vertically from and operatively coupled to the first motor. Accordingly, actuation of the first motor to cause rotation of the ball screw shaft in a first rotational direction may cause linear movement of the nut and the loading shelf in a first vertical direction and actuation of the first motor to cause rotation of the ball screw shaft in a second rotational direction opposite the first rotational direction may cause linear movement of the nut and the loading shelf in a second vertical direction opposite the first vertical direction. In addition, the transfer module further may include at least one sensor configured to detect a presence of a dialysate bag within the transfer module. The at least one sensor may comprise at least one infrared sensor.

In some embodiments, the access window may be disposed on a front lateral surface of the transfer module. Moreover, the dispensing platform may comprise at least one push arm, the at least one push arm configured to be linearly movable between the first loading position at a first horizontal rear end of the transfer module and the second dispensing position at a second horizontal front end of the transfer module via the second motor. Accordingly, the controller may be configured to cause, when the top-most dialysate bag is horizontally aligned with the access window, the second motor to move the at least one push arm from the first loading position to the second dispensing position, such that the top-most dialysate bag is advanced through the access window. In addition, the second motor may be operatively coupled to the at least one push arm via at least one nut mounted on the at least one push arm, the at least one nut threadably and slidably coupled to at least one ball screw shaft extending horizontally from and operatively coupled to the second motor. Accordingly, actuation of the second motor to cause rotation of the at least one ball screw shaft in a first rotational direction may cause linear movement of the at least one nut and the at least one push arm in a first horizontal direction and actuation of the second motor to cause rotation of the at least one ball screw shaft in a second rotational direction opposite the first rotational direction may cause linear movement of the at least one nut and the at least one push arm in a second horizontal direction opposite the first horizontal direction.

The system further may include a retractable shelf configured to transition between a retracted state where the retractable shelf defines a bottom surface of the transfer module on a rear side of the access window, and an extended state where the retractable shelf is disposed on a front side of the access window for receiving the top-most dialysate bag via the at least one push arm. Moreover, a rear end of the retractable shelf may comprise at least one roller configured to facilitate transfer of the top-most dialysate bag onto the retractable shelf via the at least one push arm. Additionally, the system may comprise at least one load cell disposed on the retractable shelf, the at least one load cell configured to detect a weight of a dialysate bag thereon.

Alternatively, in some embodiments, the access window may be disposed on a top surface of the transfer module, and the dispensing platform may comprise a dispensing shelf. The dispensing shelf may be configured to be linearly movable between the first loading position where the dispensing shelf is horizontally aligned with the second vertical position of the loading shelf and the second dispensing position where the dispensing shelf is horizontally aligned with the top surface via the second motor. In addition, the dispensing shelf may be configured to be linearly movable between a first horizontal position at a first side of the transfer module and a second horizontal position at a second side of the transfer module opposite the first side via a third motor operatively coupled to the dispensing shelf and the controller. Accordingly, the controller may be configured to cause, when the top-most dialysate bag is disposed on the dispensing shelf and the dispensing shelf is vertically aligned with the access window, the second motor to move the dispensing shelf from the first loading position to the second dispensing position, such that the top-most dialysate bag is advanced through the access window.

For example, the second motor may be operatively coupled to the dispensing shelf via a first nut mounted on the dispensing shelf, the first nut threadably and slidably coupled to a first ball screw shaft extending vertically from and operatively coupled to the second motor. Accordingly, actuation of the second motor to cause rotation of the first ball screw shaft in a first rotational direction may cause linear movement of the first nut and the dispensing shelf in a first vertical direction and actuation of the second motor to cause rotation of the first ball screw shaft in a second rotational direction opposite the first rotational direction may cause linear movement of the first nut and the dispensing shelf in a second vertical direction opposite the first vertical direction. Moreover, the third motor may be operatively coupled to the dispensing shelf via a second nut mounted on at least one of the dispensing shelf or the second motor, the second nut threadably and slidably coupled to a second ball screw shaft extending horizontally from and operatively coupled to the third motor. Accordingly, actuation of the third motor to cause rotation of the second ball screw shaft in a third rotational direction may cause linear movement of the second nut and the dispensing shelf in a first horizontal direction and actuation of the third motor to cause rotation of the second ball screw shaft in a fourth rotational direction opposite the third rotational direction may cause linear movement of the second nut and the dispensing shelf in a second horizontal direction opposite the first horizontal direction.

In addition, the transfer module further may comprise a first push arm operatively coupled to a fourth motor, the first push arm configured to be linearly movable between a third horizontal position at the first side of the transfer module and a fourth horizontal position adjacent to a first side of the access window, and a second push arm operatively coupled to a fifth motor, the second push arm configured to be linearly movable between a fifth horizontal position at the second side of the transfer module and a sixth horizontal position adjacent to the first side of the access window. Accordingly, the first and second push arms may be configured to be moved between respective horizontal positions to facilitate transfer of the top-most dialysate bag onto the dispensing shelf. Moreover, the first push arm may be configured to prevent linear movement of the top-most dialysate bag in a horizontal direction towards the first side of the transfer module beyond the first push arm, and the second push arm may be configured to prevent linear movement of the top-most dialysate bag in a horizontal direction towards the second side of the transfer module beyond the second push arm.

Additionally, the fourth motor may be operatively coupled to the first push arm via a first nut mounted on the first push arm, the first nut threadably and slidably coupled to a first ball screw shaft extending horizontally from and operatively coupled to the fourth motor. Accordingly, actuation of the fourth motor to cause rotation of the first ball screw shaft in a first rotational direction may cause linear movement of the first nut and the first push arm in a first horizontal direction and actuation of the fourth motor to cause rotation of the first ball screw shaft in a second rotational direction opposite the first rotational direction may cause linear movement of the first nut and the first push arm in a second horizontal direction opposite the first horizontal direction. Further, the fifth motor may be operatively coupled to the second push arm via a second nut mounted on the second push arm, the second nut threadably and slidably coupled to a second ball screw shaft extending horizontally from and operatively coupled to the fifth motor. Accordingly, actuation of the fifth motor to cause rotation of the second ball screw shaft in a third rotational direction may cause linear movement of the second nut and the second push arm in the first horizontal direction and actuation of the fifth motor to cause rotation of the second ball screw shaft in a fourth rotational direction opposite the third rotational direction may cause linear movement of the second nut and the second push arm in the second horizontal direction opposite the first horizontal direction.

In addition, the top surface of the transfer module may comprise a first top surface adjacent the first side of the transfer module, and a second top surface adjacent the second side of the transfer module. Accordingly, the system further may include a first load cell disposed on the first top surface, the first load cell configured to detect a weight of a dialysate bag thereon, and a second load cell disposed on the second top surface, the second load cell configured to detect a weight of a dialysate bag thereon. Moreover, the top surface may comprise a retractable cover configured to transition between a closed state where the retractable cover obstructs the access window and an open state where the retractable cover does not obstruct the access window. Additionally, the system may include a graphical user interface operatively coupled to controller, the graphical user interface comprising a display. Accordingly, the controller may be configured to cause display to display information associated with the at least one mobile column. For example, the information associated with the at least one mobile column may comprise at least one of dialysate concentrations of the plurality of dialysate bags loaded within the at least one mobile column or a quantity of dialysate bags loaded within the at least one mobile column.

### BRIEF DESCRIPTION OF THE DRAWINGS

FIG. 1 illustrates an exemplary dialysis circuit in accordance with some embodiments.

FIGS. 2A to 2D illustrate various exemplary connector types.

FIG. 3 illustrates an exemplary automated connector system in accordance with some embodiments.

FIGS. 4A to 4C illustrate an exemplary case for automated connection of a dialysate bag and a solution line.

FIGS. 4D to 4F illustrate an exemplary moving platform of the case of FIGS. 4A to 4C.

FIG. 4G illustrates an exemplary slide table in accordance with some embodiments.

FIGS. 5A to 5K illustrate exemplary method steps for automated connection via the case of FIGS. 4A to 4C.

FIGS. 6A to 6C illustrate an exemplary case for automated connection of a transfer set and a patient line.

FIGS. 6D to 6F illustrate an exemplary moving platform of the case of FIGS. 6A to 6C.

FIGS. 7A to 7O illustrate exemplary method steps for automated connection via the case of FIGS. 6A to 6C.

FIGS. 8A and 8B illustrate an exemplary case for automated clamping of unconnected solution lines.

FIGS. 9A to 9E illustrate exemplary method steps for automated clamping via the case of FIGS. 8A and 8B.

FIG. 10A illustrates an exemplary case for automated connection of alternative solution lines connector types in accordance with some embodiments.

FIGS. 10B and 10C illustrate an exemplary moving platform of the case of FIG. 10A.

FIGS. 11A to 11L illustrate exemplary method steps for automated connection via the case of FIG. 10A.

FIGS. 12A to 12D illustrate an exemplary case for automated effluent sampling.

FIGS. 12E to 12H illustrate an exemplary effluent sampling system of the case of FIGS. 12A to 12D.

FIGS. 12I and 12J illustrate an exemplary interposer of the effluent sampling system of FIGS. 12E to 12H.

FIGS. 12K and 12L illustrate an exemplary test strip holder of the effluent sampling system of FIGS. 12E to 12H.

FIGS. 13A to 13E illustrate exemplary method steps for automated effluent sampling via the case of FIGS. 12A to 12L.

FIGS. 14A to 14C illustrate an exemplary compact dispensing system for automated storage and dispensing of dialysate bags in accordance with some embodiments.

FIGS. 14D to 14F illustrate an exemplary magazine cart of the compact dispensing system of FIGS. 14A to 14C.

FIGS. 14G to 14I illustrate an exemplary transfer module of the compact dispensing system of FIGS. 14A to 14C.

FIGS. 15A to 15O illustrate exemplary method steps for automated storage and dispensing of dialysate bags via the compact dispensing system of FIGS. 14A to 14I.

FIG. 16A illustrates an alternative exemplary compact dispensing system in accordance with some embodiments.

FIGS. 16B to 16E illustrate an exemplary transfer module of the compact dispensing system of FIG. 16A.

FIGS. 17A to 17D illustrate exemplary method steps for automated dispensing of dialysate bags via the compact dispensing system of FIG. 16A.

### DETAILED DESCRIPTION

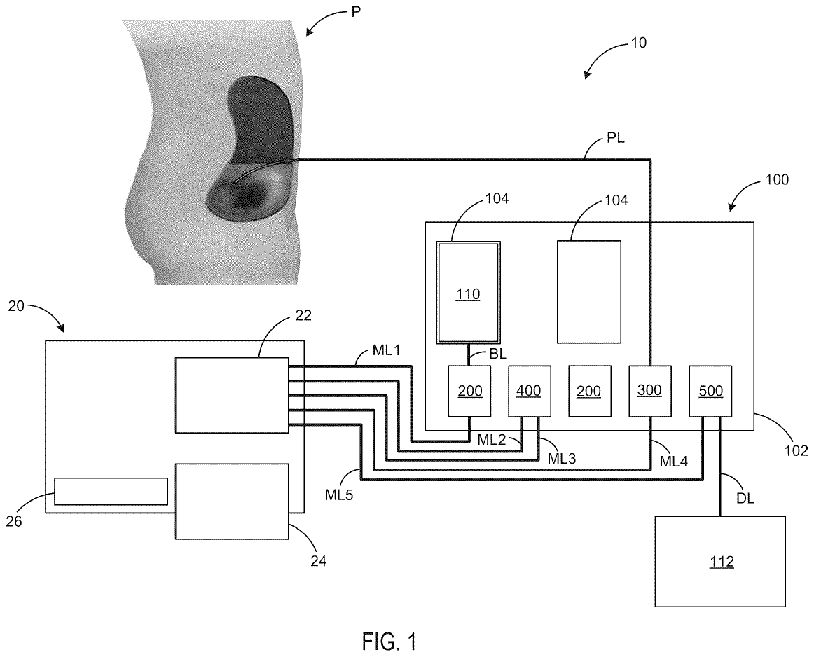

Embodiments of this technology are directed to exemplary systems and methods for automatically connecting various dialysis line connector types of conventional, commercially available dialysis lines, e.g., soft plastic tubes, together under sterile conditions to complete a dialysis circuit. As shown in FIG. 1, dialysis circuit 10 may include automated dialysis line connection system 100 comprising a series of cases, e.g., cases 200, 400, 500, each sized and shaped to receive at least the line connector portions of two dialysis lines and including a mechanism for automatically connecting the line connector portions to thereby fluidically couple the two dialysis lines together. The system further may include one or more cases, e.g., case 300, for automated clamping of unused lines, e.g., the lines of extra cycler solution lines of the cassette. The appropriate case may be selected based on the line connector types of the dialysis lines to be connected, as described in further detail below. In addition, the motors described herein may be a stepper or servo motor, and the cases may be battery powered, e.g., by rechargeable lithium-ion or lithium polymer batteries, and/or electrically via an electric power outlet.

As shown in FIG. 1, the various dialysis lines of dialysis circuit 10 may include, for example, the dialysis bag administration port BL fluidically coupled to a source of dialysate, e.g., dialysis bag 110, the transfer set PL fluidically coupled to and extending from the body of patient P, e.g., the peritoneal/abdominal cavity, the drain bag line DL fluidically coupled to an external reservoir for collecting fluid drained from the patient's body, e.g., drain bag 112, as well as lines operatively coupled pump 22 of cycler 20 for pumping fluid throughout dialysis circuit 10 such as, for example, one or more cycler solution lines ML1, ML2, ML3, for fluidically coupling cycler 20 to one or more dialysis bags 110, e.g., via the associated dialysis bag administration port BL, cycler patient line ML4 for fluidically coupled cycler 20 to the patient's body, e.g., via transfer set PL, and cycler drain line ML5 for fluidically coupled cycler 20 to drain bag 112, e.g., via drain bag line DL.

The free ends of each of the cycler solution lines ML1, ML2, ML3, cycler patient line ML4, and cycler drain line ML5 may be removably coupled to cassette 24 of cycler 20, prior to insertion within the respective case by the user. As will be understood by a person having ordinary skill in the art, cycler 20 may include less or more than three cycler solution lines, as shown in FIG. 1. Cycler 20 may be a conventional cycler configured to be operatively coupled to automated dialysis line connection system 100, e.g., to transmit electrical signals therebetween, or, alternatively, may be custom built and integrated with automated dialysis line connection system 100. Moreover, the operations of cycler 20 and automated dialysis line connection system 100 may be controlled by the user via user interface 26 of cycler 20.

Each of the various conventional dialysis lines may include a unique line connector type for coupling the dialysis line to another dialysis line having a different or same line connector type, which may be selected based on the conventional dialysis line manufacturer. For example, each of the dialysis lines may include any one of line connector types C1-C4, as described in further detail below, such that the appropriate case for automated line connection may be selected based on the line connector type rather than the specific type of dialysis line. Thus, while the specific dialysis lines may be described herein has having a specific line connector type, e.g., for brevity to illustrate a specific automated connection mechanism, as will be understood by a person having ordinary skill in the art, the same case may be used to connect different dialysis lines than described herein based on the line connector type of the lines to be connected.

As described herein, for illustrative purposes, case 200 may be configured for automatically fluidically coupling a cycler solution line, e.g., any one of cycler solution lines ML1, ML2, ML3, and dialysis bag administration port BL, case 300 may be configured for automatically fluidically coupling cycler patient line ML4 with transfer set PL, case 400 may be configured for automatically clamping any unused, extra cycler solution lines, e.g., cycler solution lines ML2, ML3, and case 500 may be configured for automatically fluidically coupling cycler drain line ML5 with drain bag line DL. As will be understood by a person having ordinary skill in the art, each of the cases described above may be used for automatically connecting different dialysis lines based on the connector type of the dialysis line and, thus, one or more of the cases may be optional based on the connector type available. In addition, while the cycler solution lines are referred to herein as “solution lines,” the same lines, which are fluidically coupled to transfer set PL, are used for bidirectional flow between cycler 20 and the patient's body, e.g., for transferring fluid into the patient and for draining fluid out of the patient, as described in further detail below.

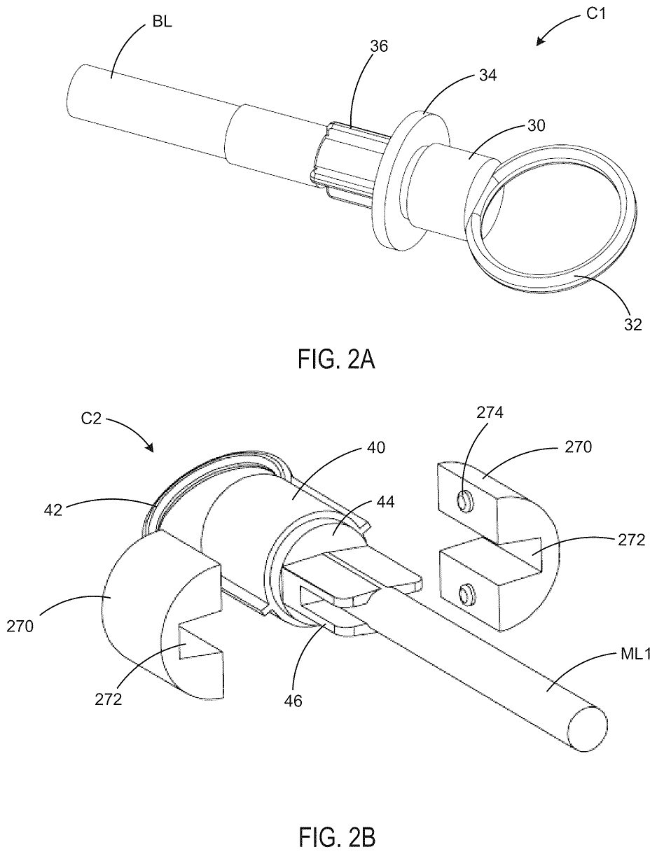

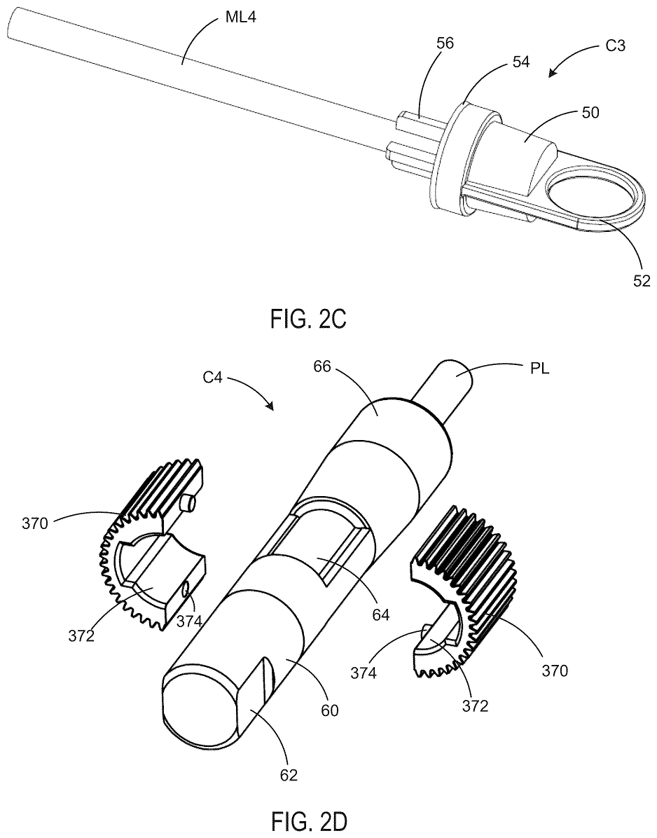

Referring now to FIGS. 2A to 2D, various line connector types C1-C4 are described. FIG. 2A illustrates connector type C1. As described above, any of the other dialysis lines described herein may include connector type C1; however, for illustrative purposes, FIG. 2A illustrates bag administration port BL having connector type C1. As shown in FIG. 2A, connector C1 is disposed at the free end of bag administration port BL and may include removable cap 30 having pull ring 32 sized and shaped to facilitate removal of cap 30, e.g., via a pulling force. Connector C1 further may include lip 34 extending circumferentially and radially outward from a longitudinal axis of connector C1, as well as one or more ridges 36 extending longitudinally and radially outward along the circumference of connector C1, proximal to lip 34.

FIG. 2B illustrates connector type C2. As described above, any of the other dialysis lines described herein may include connector type C2; however, for illustrative purposes, FIG. 2B illustrates a cycler solution line, e.g., cycler solution line ML1, having connector type C2. As shown in FIG. 2B, connector C2 is disposed at the free end of cycler solution line ML1 and may include removable cap 40 having pull ring 42 sized and shaped to facilitate removal of cap 40, e.g., via a pulling force. Connector C2 further may include lip 44 extending circumferentially and radially outward from a longitudinal axis of connector C2, as well as flat retention portion 46 extending longitudinally along a length of connector C2, proximal to lip 44. Automated dialysis line connection system 100 further may include retainer gear 270 configured to releasably grip flat retention portion 46, to thereby transmit rotary and linear motion from retainer gear 270 to connector C2. For example, as shown in FIG. 2B, retainer gear 270 may be formed of two gear halves configured to be releasably coupled together, e.g., via engagers 274, each of the two gear halves having inner cavity 272 sized and shaped to receive a portion of flat retention portion 46 therein. Engagers 274 may be configured to releasably couple the two gear halves of retainer gear 270 via, e.g., interference fit, magnetic connection, etc. Moreover, the outer surface of retainer gear 270 may comprise a textured surface, e.g., teeth, configured to provide additional friction to transmit rotary motion to connector C2, as described in further detail below.

FIG. 2C illustrates connector type C3. As described above, any of the other dialysis lines described herein may include connector type C3; however, for illustrative purposes, FIG. 2C illustrates cycler patient line ML4 having connector type C3. As shown in FIG. 2C, connector C3 is disposed at the free end of cycler patient line ML4 and may include removable cap 50 having pull ring 52 sized and shaped to facilitate removal of cap 50, e.g., via a pulling force. Connector C3 further may include lip 54 extending circumferentially and radially outward from a longitudinal axis of connector C3, as well as one or more ridges 56 extending longitudinally and radially outward along the circumference of connector C3, proximal to lip 54.

FIG. 2D illustrates connector type C4. As described above, any of the other dialysis lines described herein may include connector type C4; however, for illustrative purposes, FIG. 2D illustrates transfer set PL having connector type C4. As shown in FIG. 2D, connector C4 is disposed at the free end of transfer set PL and may include cap portion 60 having one or more gripping portions 62 sized and shaped to be facilitate gripping of cap portion 60. For example, gripping portions 62 may include a flat surface disposed on and indented within the circumferential outer surface of cap portion 60. In addition, connector C4 may include retention portion 64 rotatably coupled to cap portion 60, and having an indented surface extending radially inward from the outer surface of connector C4 and longitudinally along a length of connector C4, proximal to cap portion 60. Automated dialysis line connection system 100 further may include retainer gear 370 configured to releasably grip the indented surface of retention portion 64, to thereby transmit rotary and linear motion from retainer gear 370 to retention portion 64 of connector C4. For example, as shown in FIG. 2D, retainer gear 370 may be formed of two gear halves configured to be releasably coupled together, e.g., via engagers 374, each of the two gear halves having inner cavity 372 sized and shaped to receive a portion of the indented surface of retention portion 64 therein. Engagers 374 may be configured to releasably couple the two gear halves of retainer gear 370 via, e.g., interference fit, magnetic connection, etc. Moreover, the outer surface of retainer gear 370 may comprise a textured surface, e.g., teeth, configured to provide additional friction to transmit rotary motion to connector C4, as described in further detail below. As shown in FIG. 2D, connector C4 further may include lock portion 66 rotatably coupled to retention portion 64 between a locked state where the twist lock of connector C4 prevents fluid flow therethrough and an unlocked state where the twist lock of connector C4 permits fluid flow therethrough.

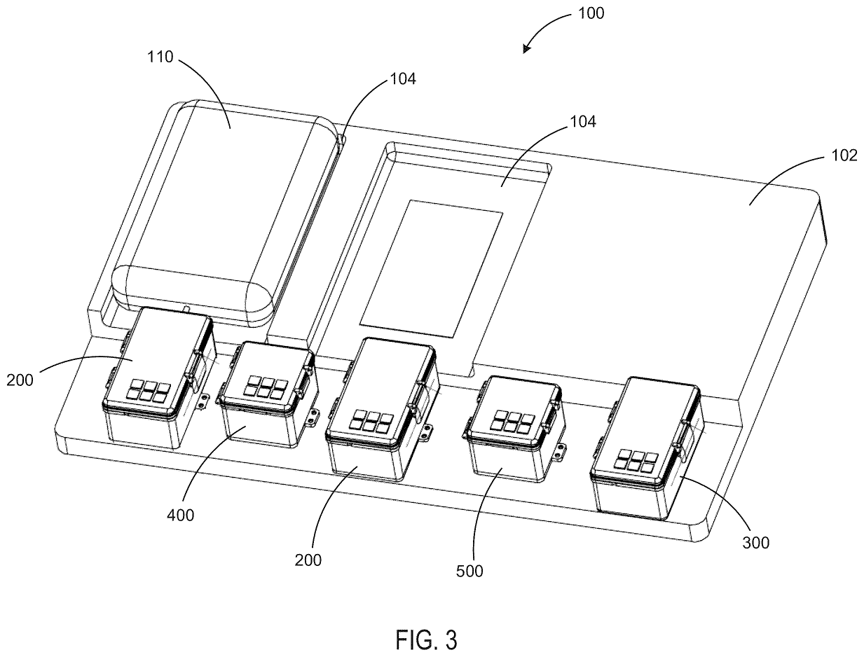

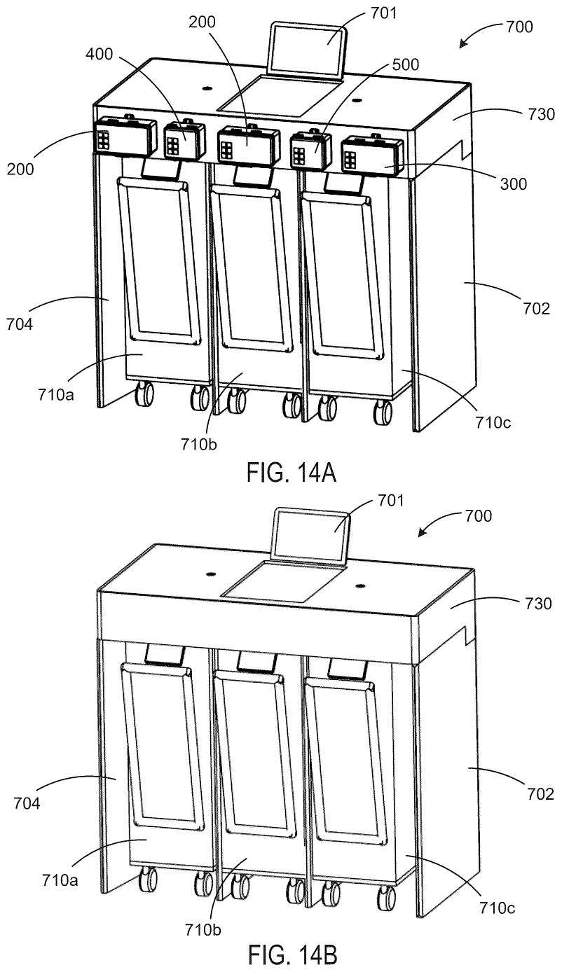

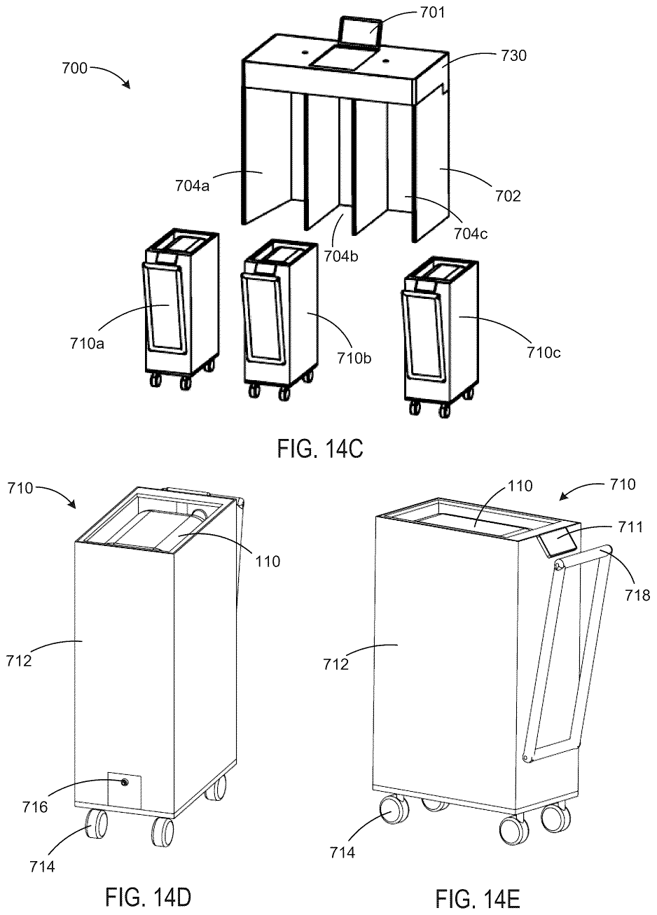

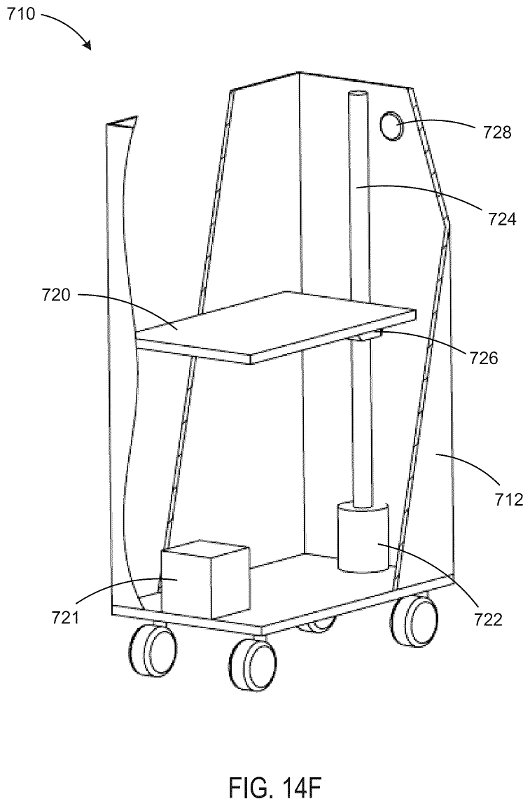

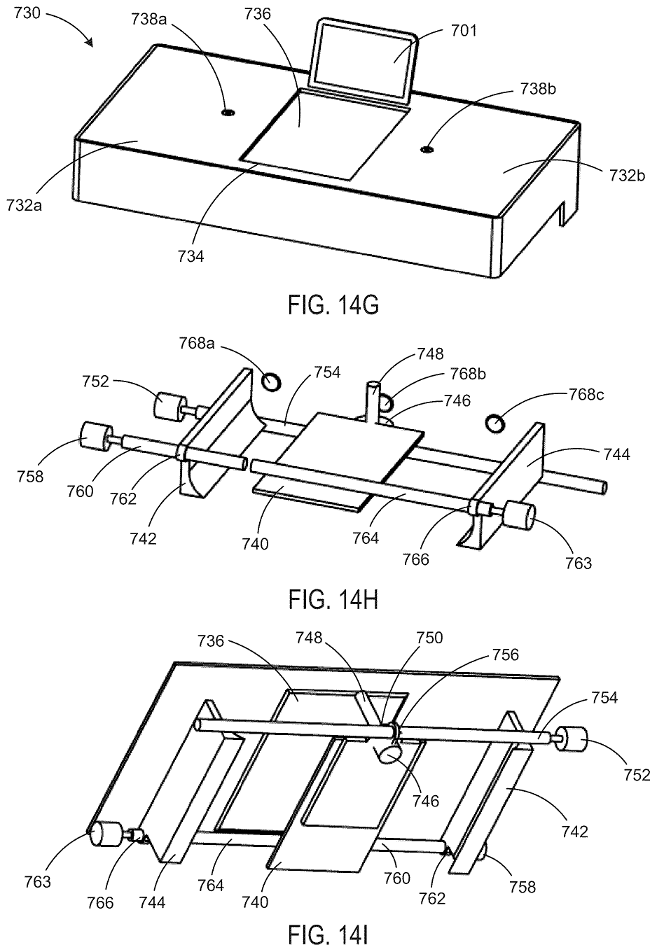

Referring now to FIG. 3, automated dialysis line connection system 100 is provided. As shown in FIG. 3, automated dialysis line connection system 100 may include platform 102 configured to support one or more of the cases described herein, e.g., cases 200, 300, 400, 500. Platform 102 further may include one or more bag slots 104, each sized and shaped to receive a dialysis bag, e.g., dialysis bag 110, comprising dialysate. Accordingly, an unused dialysis bag may be disposed in a bag slot with its corresponding bag line uncoupled from the dialysis circuit until it is needed. As will be understood by a person having ordinary skill in the art, while FIG. 3 illustrates platform 102 having two bag slots 104, platform 102 may include less or more than two bag slots. In addition, while FIG. 3 illustrates a single platform 102, automated dialysis line connection system 100 may comprise more than one platform. For example, multiple platforms may be stacked vertically, such that one or more designated cases may be disposed on a different platform, e.g., case 500 for fluidically coupling cycler drain line ML5 with drain bag line DL may be disposed on a lower platform, which may include a bag slot sized and shaped to receive drain bag 112. Additionally, or alternatively, the platform for supporting dialysate bags and the cases described herein may be integrated with a modular loading and dispensing system, as described in further detail below with regard to FIGS. 14A to 17D.

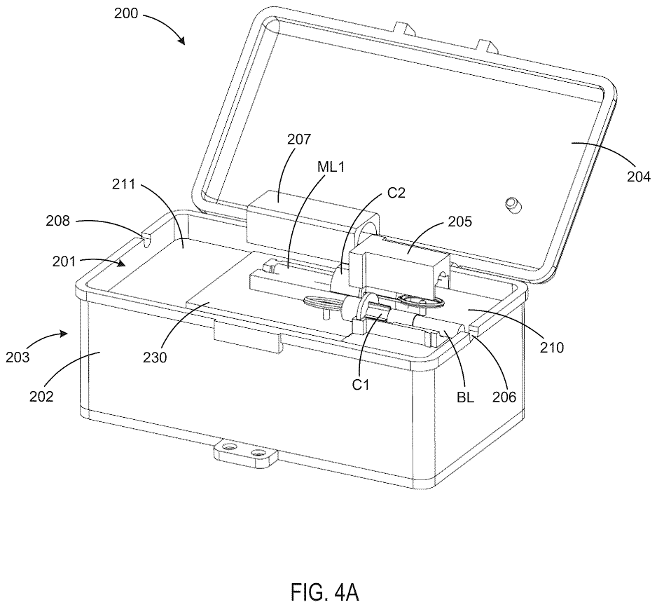

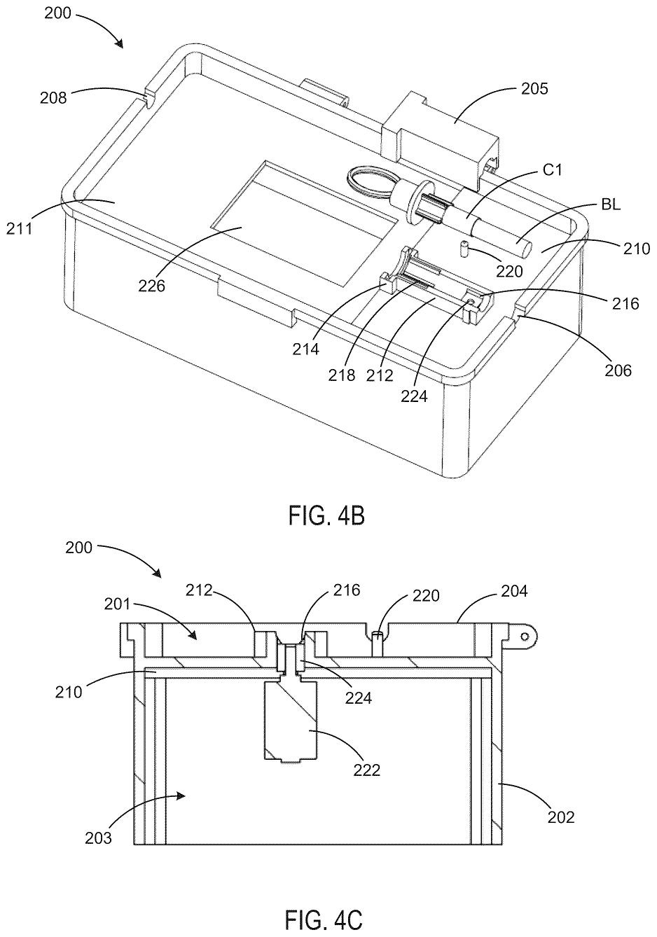

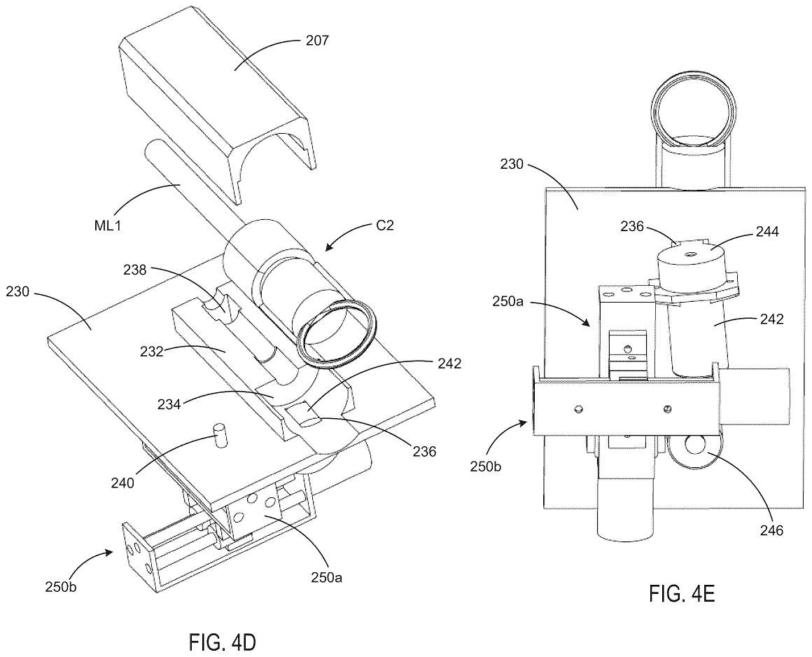

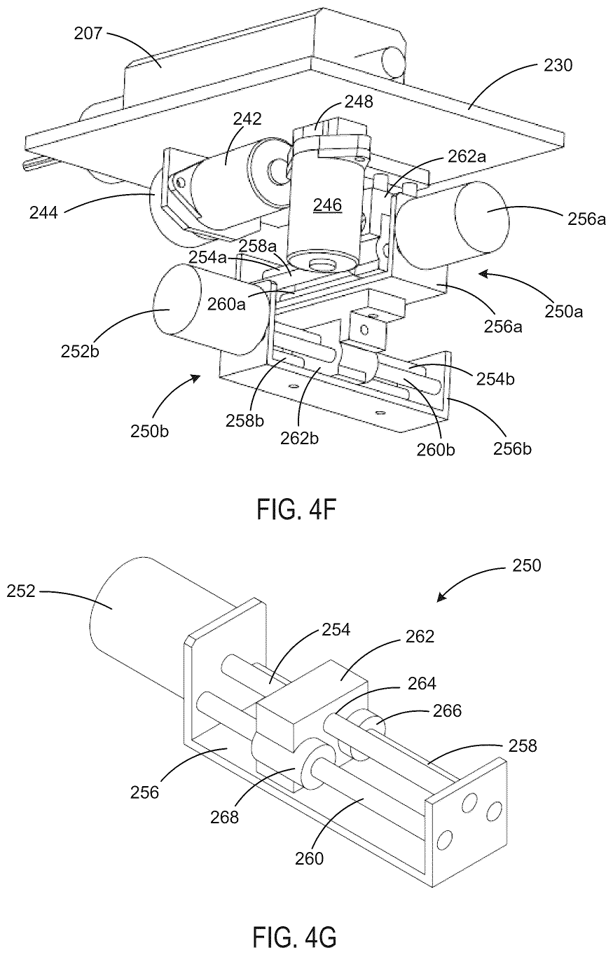

Referring now to FIGS. 4A to 4F, an exemplary case for automated connection of a dialysis line having connector type C1, e.g., bag administration port BL, with a dialysis line having connector type C2, e.g., cycler solution line ML1, is provided. Accordingly, case 200 may be selected for automatically coupling connector type C1 to connector type C2 to thereby fluidically couple bag administration port BL with cycler solution line ML1, and accordingly, dialysis bag 110 with cycler 20 to permit fluid flow therebetween. As shown in FIGS. 4A to 4C, case 200 may include housing 202, and lid 204 configured to enclose housing 202. Housing 202 may include first window 206 and second window 208, e.g., along the upper edge of housing 202, each sized and shaped to receive the tubing of a respective dialysis line therein in a manner that does not disrupt fluid flow therethrough, such that upon closure of lid 204, the respective dialysis lines will be maintained within first window 206 and second window 208. Closing lid 204 will block pathogens from contacting the dialysis lines and connectors, thereby maintaining sterility.

Housing 202 may include horizontally extending platform 211 disposed therein that divides the interior of housing 202 upper chamber 201 and lower chamber 203. For example, the connectors and associated dialysis lines may be positioned within upper chamber 201, while the moving mechanisms and electronic controls of case 200 may be disposed within lower chamber 203. Platform 211 may include fixed platform 210 that is fixed relative to housing 202, and opening 226 sized and shaped to receive at least a portion of movable platform 230 therethrough, such that movable platform 230 may be actuated to move relative to platform 211, and accordingly, fixed platform 210. The upper surface of fixed platform 210 may include slot 212 configured to releasably receive connector C1 therein, and further may include cover 205 configured to be removably disposed over slot 212 to thereby maintain and protect connector C1 within slot 212 during operation of case 200. Preferably, the connector of bag administration port BL is positioned on the fixed platform as the length of the tubing of bag lines are typically relatively shorter than other dialysis lines and should remain stationary during automated connection. Slot 212 may include lip cavity 214 sized and shaped to receive lip 34 of connector C1 therein in a manner that prevents axial movement of connector C1 relative to slot 212, and accordingly, fixed platform 210. In addition, slot 212 may include plurality of grooves 218 having a geometry that corresponds with the geometry of ridges 36 of connector C1, such that ridges 36 may engage with plurality of grooves 218 when connector C1 in inserted within slot 212, to thereby prevent rotation of connector C1 relative to slot 212.

As shown in FIG. 4B, slot 212 further may include channel 216 sized and shaped to permit break block 224 to pass therethrough for engagement with the seal of bag administration port BL. As shown in FIG. 4C, break block 224 may be operatively coupled to motor 222, such that upon actuation of motor 222, break block 224 may be configured to move vertically between a first position where break block 224 is not engaged with the seal of bag administration port BL, and a second position where break block 224 is engaged with the seal of bag administration port BL, to thereby break the frangible seal and permit fluid flow between bag administration port BL and connector C1. For example, the interior of break block 224 may include a threaded surface configured to envelop the threaded axel of motor 222, such that upon rotation of the threaded axel via motor 222, break block 224 may slide along the threaded axel within channel 216 between the first and second positions. Accordingly, channel 216 may be positioned along slot 212 such that channel 216, and accordingly break block 224, are aligned with the seal of bag administration port BL when connector C1 is disposed within slot 212. In addition, fixed platform 210 may include cap hook 220 protruding upwardly from the upper surface of fixed platform 210, and configured to be received within the pull ring of the connector configured to be coupled to movable platform 230, e.g., pull ring 42 of removable cap 40 of connector C2.

Referring now to FIGS. 4D to 4G, movable platform 230 is provided. As shown in FIGS. 4D to 4F, the upper surface of movable platform 230 may include slot 232 configured to releasably receive connector C2 therein, and further may include cover 207 configured to be removably disposed over slot 232 to thereby maintain and protect connector C2 within slot 232 during operation of case 200. Slot 232 may include cavity 234 sized and shaped to receive lip 44 of connector C2 and retainer gear 270 therein in a manner that prevents axial movement of connector C2 relative to slot 232, and accordingly, movable platform 230. For example, the proximal wall of cavity 234 may abut against the proximal side of lip 44 to thereby prevent proximal movement of connector C2 relative to slot 232.

In addition, as shown in FIG. 4D, slot 232 further may include opening 236 sized and shaped to permit at least a portion of angled, rotatable drive gear 244 to extend therethrough for engagement with retainer gear 270. As shown in FIG. 4E, drive gear 244 may be operatively coupled to motor 242, such that upon actuation of motor 242, drive gear 244 may be configured to rotate about its longitudinal axis, at least partially through opening 236 of slot 232. Moreover, as shown in FIG. 4E, motor 242 and drive gear 244 may be coupled to the bottom surface of movable platform 230 at angle relative to slot 232, such that drive gear 244 contacts retainer gear 270 through opening 236 at an incline angle. Accordingly, upon rotation of drive gear 244 via motor 242, the engagement between drive gear 244 and retaining gear 270 causes rotational and linear movement of retainer gear 270, and accordingly, connector C2 relative to slot 232. Thus, rotation of drive gear 244 in a first direction (e.g., clockwise) causes rotation of retainer gear 270 in a second direction (e.g., counter-clockwise) opposite the first direction and linear movement retainer gear 270 in a distal direction relative to slot 232, and rotation of drive gear 244 in the second direction (e.g., counter-clockwise) causes rotation of retainer gear 270 in the first direction (e.g., clockwise) opposite the second direction and linear movement retainer gear 270 in a proximal direction relative to slot 232.

Referring again to FIG. 4D, slot 232 further may include channel 238 sized and shaped to permit clamp block 248 to pass therethrough for engagement with the tubing of cycler solution line ML1. As shown in FIG. 4F, clamp block 248 may be operatively coupled to motor 246, such that upon actuation of motor 246, clamp block 248 may be configured to move vertically between a first position where clamp block 248 is not engaged with the tubing of cycler solution line ML1, and a second position where clamp block 248 is engaged with the tubing of cycler solution line ML1, to thereby clamp the tubing and prevent fluid flow between cycler solution line ML1 and connector C2. For example, the interior of clamp block 248 may include a threaded surface configured to envelop the threaded axel of motor 246, such that upon rotation of the threaded axel via motor 246, clamp block 248 may slide along the threaded axel within channel 238 between the first and second positions. Accordingly, channel 238 may be positioned along slot 232 such that channel 238, and accordingly clamp block 248, are aligned with tubing of cycler solution line ML1 when connector C2 is disposed within slot 232. In addition, moveable platform 230 may include cap hook 240 protruding upwardly from the upper surface of moveable platform 230, and configured to be received within the pull ring of the connector configured to be coupled to fixed platform 210, e.g., pull ring 32 of removable cap 30 of connector C1.

Movable platform 230 may be configured to be movable relative to platform 211, and accordingly, fixed platform 210, e.g., along the horizontal X-axis and horizontal Y-axis, via first and second slide tables 250a, 250b. For example, first slide table 250a may be configured to move movable platform 230 along the X-axis, and second slide table 250b may be configured to move movable platform 230 along the Y-axis. First slide table 250a and second slide table 250b may be substantially structurally identical and, thus, for brevity, will be referred to collectively herein as slide table 250 with regard to FIG. 4G. As shown in FIG. 4G, slide table 250 may include frame 256 having one or more smooth support rods, e.g., support rods 258, 260, fixed thereto and extending longitudinally along the length of frame 256, motor 252 operatively coupled to threaded axel 254, e.g., a lead screw, which also extends longitudinally along the length of frame 256, parallel to support rods 258, 260, and slide block 262 threadably and slidably engaged with threaded axel 254 via threaded channel 264 and slidably engaged with support rods 258, 260 via non-threaded channels 266, 268, respectively.

For example, the inner surface of threaded channel 264 of slide block 262 may include a threaded surface configured to envelop threaded axel 254 of motor 252, such that upon rotation of threaded axel 254 via motor 252, the engagement between threaded channel 264 of slide block 262 and threaded axel 254, as well as the slidable engagement between non-threaded channels 266, 268 and support rods 258, 260, causes slide block 262 to move along threaded axel 254 and support rods 258, 260. As shown in FIG. 4F, the bottom surface of frame 256a of slide table 250a may be fixedly coupled to the upper surface of slide block 262b of slide table 250b, such that movement of slide block 262b along threaded axel 254b and support rods 258b, 260b causes a corresponding movement of slide table 250a relative to slide table 250b. Moreover, frame 256b of slide table 250b may be fixedly coupled to housing 202 of case 200 and slide block 262a of slide table 250a may be fixedly coupled to moveable platform 230, such that movement of slide block 262a along threaded axel 254a and support rods 258a, 260a causes a corresponding movement of movable platform 230 relative to frame 256a, and accordingly, slide table 250b, and movement of slide block 262b along threaded axel 254b and support rods 258b, 260b causes a corresponding movement of slide table 250a, and accordingly, moveable platform 230 relative to frame 256b, and accordingly, housing 202.

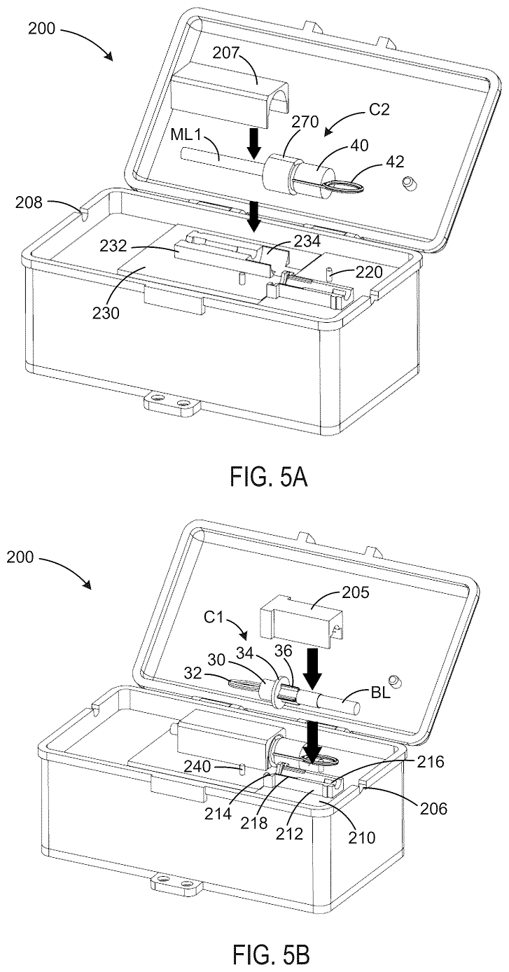

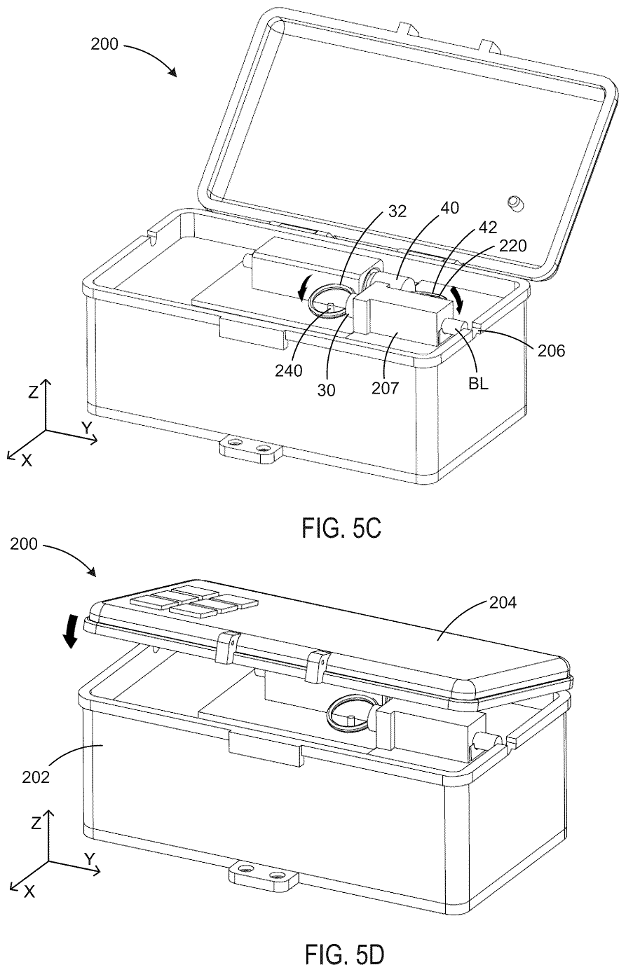

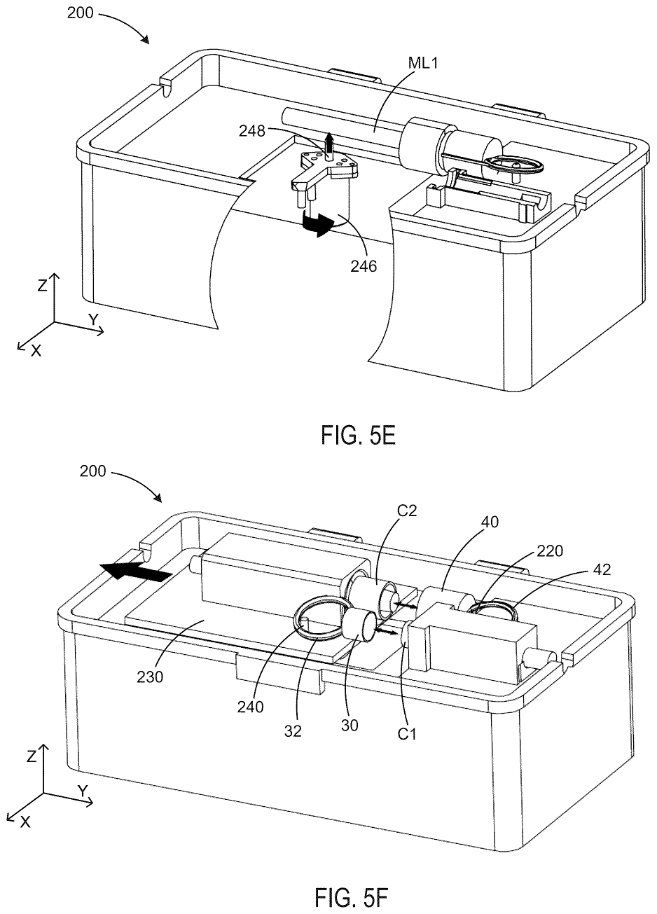

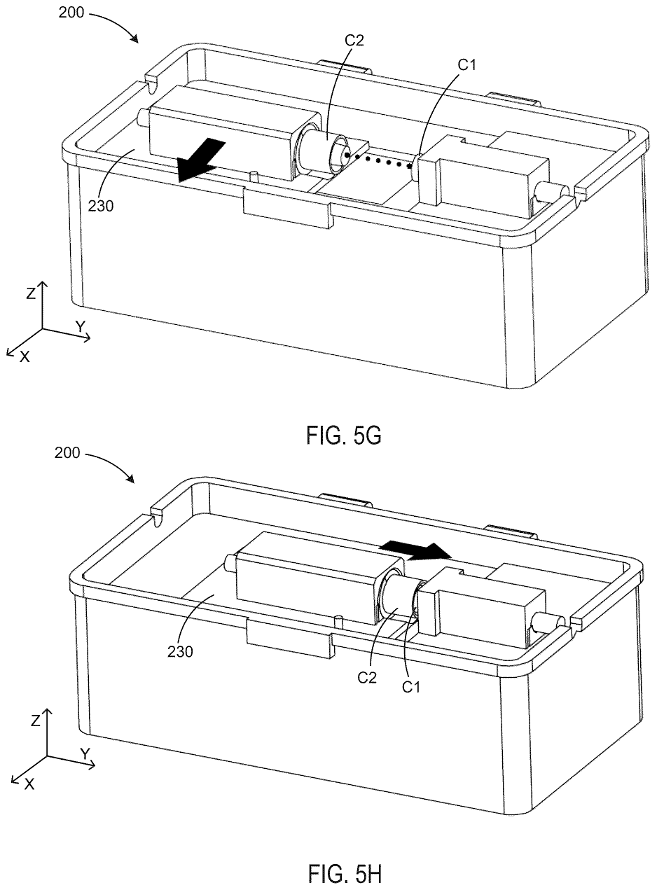

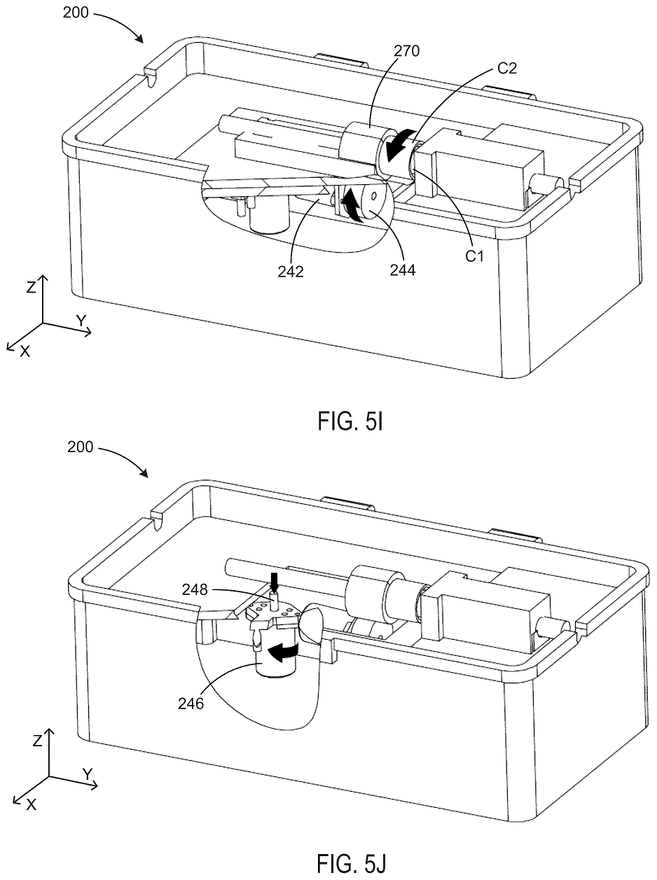

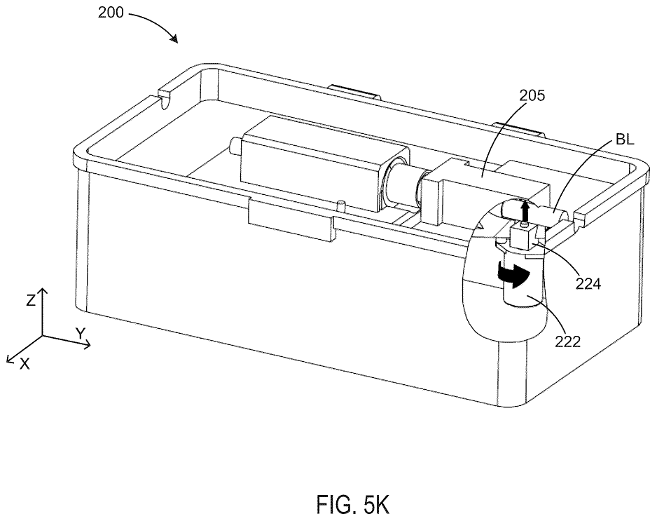

Referring now to FIGS. 5A to 5K, exemplary method steps for automated connection of connector type C1 and connector type C2 via case 200 to thereby fluidically couple bag administration port BL and cycler solution line ML1 is provided. As shown in FIG. 5A, the user may insert connector C2 into slot 232 of movable platform 230 such that retainer gear 270 is disposed within cavity 234, and may position cover 207 over connector C2 within slot 232. In addition, the user may position the tubing of cycler solution line ML1 within window 208. As shown in FIG. 5B, the user may insert connector C1 into slot 212 of fixed platform 210 such that lip 34 is disposed within cavity 214 and ridges 36 are disposed within grooves 218 of slot 212, and may position cover 205 over connector C1 within slot 212. In addition, the user may position tubing of bag administration port BL within window 206. The user may then wrap pull ring 42 of cap 40 and pull ring 32 of cap 30 over hook 220 and hook 240, respectively, such that hook 220 and hook 240 are received within and extend upwardly through pull ring 42 and pull ring 32, as shown in FIG. 5C. The user may then close lid 204 of case 200, as shown in FIG. 5D. No further actions by the user are required for the automated connection of connector C1 and connector C2, and accordingly, bag administration port BL and cycler solution line ML1 via case 200 beyond this stage.

Accordingly, upon actuation of case 200, e.g., via a controller of automated dialysis line connection system 100, as shown in FIG. 5E, motor 246 may be automatically actuated in a first direction to cause clamp block 248 to slide from its first disengaged position along the vertical Z-axis within channel 238 to its second engaged position where it clamps the tubing of cycler solution line ML1 to thereby prevent fluid flow between cycler solution line ML1 and connector C2. Next, as shown in FIG. 5F, movable platform 230 may then be automatically moved in a first direction from a first position along the horizontal Y-axis to a second position along the horizontal Y-axis away from fixed platform 210 (e.g., via slide table 250a), such that the engagement between hook 220 and pull ring 42 and the engagement between hook 240 and pull ring 32 causes hook 220 to apply a force against pull ring 42 and hook 240 to apply a force against pull ring 32, respectively, as movable platform 230, and accordingly, connector C2 moves away from fixed platform 210, thereby pulling cap 40 off of connector C2 and pulling cap 30 off of connector C1.

With the respective caps removed, movable platform 230 may then be automatically moved in a first direction from a first position along the horizontal X-axis to a second position along the horizontal X-axis (e.g., via slide table 250b), as shown in FIG. 5G, to thereby align connector C2 coaxially with connector C1. As shown in FIG. 5H, movable platform 230 may then be automatically moved in a second direction from the second position along the horizontal Y-axis to a third position along the horizontal Y-axis toward fixed platform 210 (e.g., via slide table 250a) such that connector C1 is engaged with connector C2. In some embodiments, the third position along the horizontal Y-axis may be the same as the first position along the horizontal Y-axis. Next, as shown in FIG. 5I, motor 242 may then be automatically actuated in a first direction to cause drive gear 244 to rotate in the first direction, such that the engagement between drive gear 244 and retainer gear 270 at the incline angle causes retainer gear 270, and accordingly, connector C2 to rotate in a second direction opposite the first direction of rotation of drive gear 244, while simultaneously moving linearly along the Y-axis towards fixed platform 210, thereby tightly connecting connector C2 to connector C1, e.g., via a screw type engagement between connector C2 to connector C1. In some embodiments, connector type C1 and connector type C2 may both include double-start threads, such that each may only need to rotate 180 degrees for full attachment and/or removal.

Upon connection of connector C2 to connector C1, as shown in FIG. 5J, motor 246 may then be automatically actuated in a second direction to cause clamp block 248 to slide from its second engaged position along the vertical Z-axis within channel 238 to its first disengaged position where it unclamps the tubing of cycler solution line ML1 to thereby permit fluid flow between cycler solution line ML1 and connector C2. Next, as shown in FIG. 5K, motor 222 may then be then automatically actuated in a first direction to cause break block 224 to slide from its first disengaged position along the vertical Z-axis within channel 216 to its second engaged position where it engages with and breaks the seal of bag administration port BL to thereby permit fluid flow between bag administration port BL and connector C1. Motor 222 may then be automatically actuated in a second direction to cause break block 224 to slide from its second engaged position along the vertical Z-axis within channel 216 to its first disengaged position.

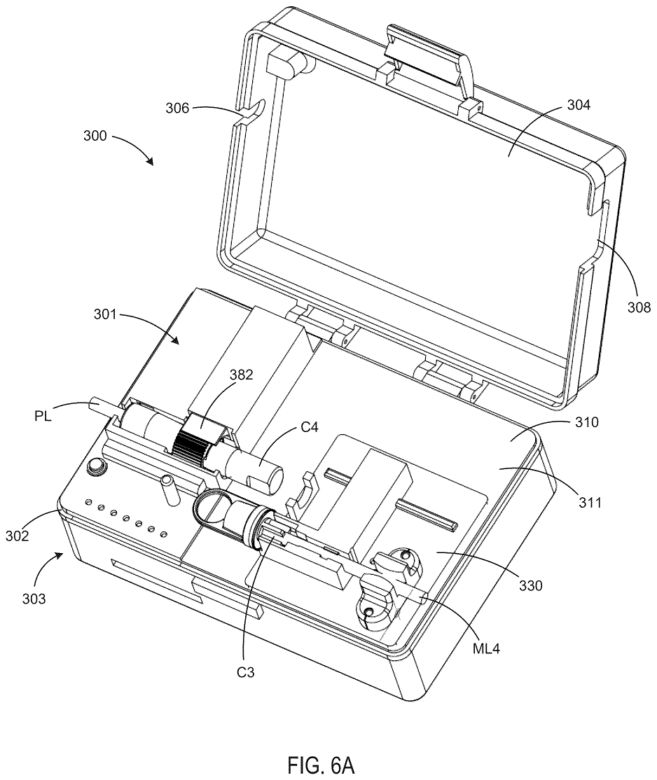

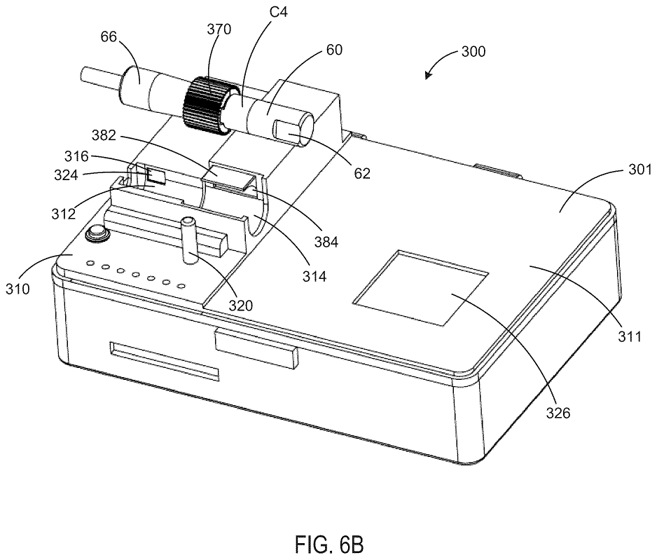

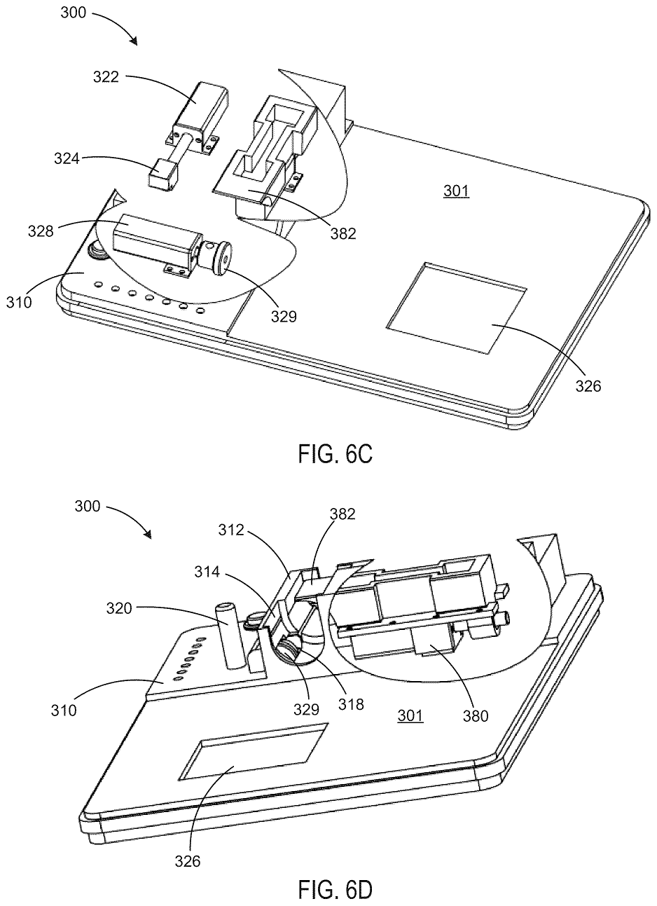

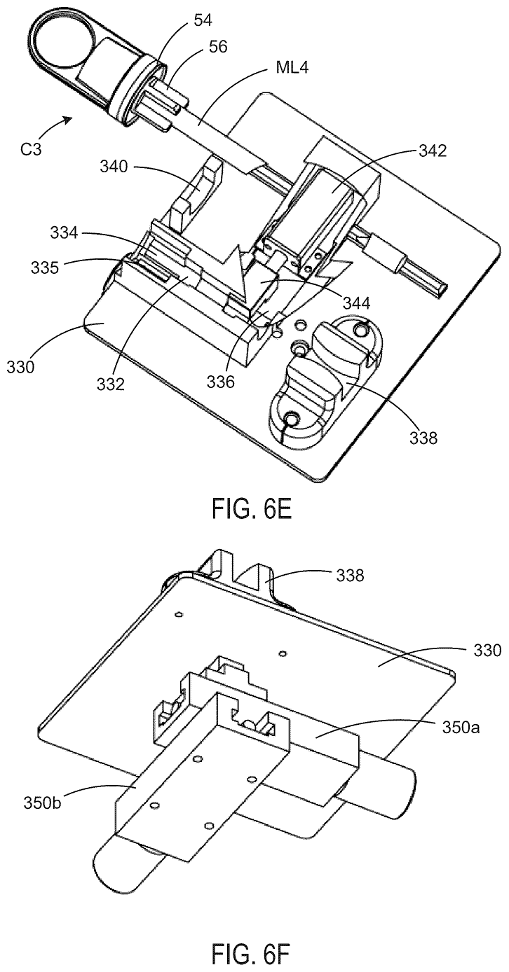

Referring now to FIGS. 6A to 6E, an exemplary case for automated connection of a dialysis line having connector type C3, e.g., cycler patient line ML4, with a dialysis line having connector type C4, e.g., transfer set PL, is provided. Accordingly, case 300 may be selected for automatically coupling connector type C4 to connector type C3 to thereby fluidically couple cycler patient line ML4 with transfer set PL, and accordingly, cycler 20 with patient body P to permit fluid flow therebetween. As shown in FIGS. 6A to 6C, case 300 may include housing 302, and lid 304 configured to enclose housing 302. Lid 304 may include first window 306 and second window 308, e.g., along the walls of lid 304, each sized and shaped to receive the tubing of a respective dialysis line therein in a manner that does not disrupt fluid flow therethrough, such that upon closure of lid 304, the respective dialysis lines will be maintained within first window 306 and second window 308.

Housing 302 may include horizontally extending platform 311 disposed therein that divides the interior of housing 302 upper chamber 301 and lower chamber 303, to thereby maintain a barrier that prevents pathogens from entering the upper chamber (which contains the dialysis lines) from the lower chamber. For example, the connectors and associated dialysis lines may be positioned within upper chamber 301, while the moving mechanisms and electronic controls of case 300 may be disposed within lower chamber 303. Platform 311 may include fixed platform 310 that is fixed relative to housing 302, and opening 326 sized and shaped to receive at least a portion of movable platform 330 therethrough, such that movable platform 330 may be actuated to move relative to platform 311, and accordingly, fixed platform 310. The upper surface of fixed platform 310 may include slot 312 configured to releasably receive connector C4 therein in a manner that prevents axial movement of connector C4 relative to slot 312, and accordingly, fixed platform 310. For example, the distal wall of slot 312 may abut against the distal side of lock portion 66 to thereby prevent distal movement of connector C4 relative to slot 312. Slot 312 may include cavity 314 sized and shaped to receive retainer gear 370 of connector C4 therein, and fixed platform 310 further may include lock cover 382 configured to slide over retainer gear 370 to thereby maintain connector C4 within slot 314 during operation of case 300.

As shown in FIG. 6B, slot 312 further may include channel 316 sized and shaped to permit lock block 324 to pass therethrough for engagement with lock portion 66 of connector C4. As shown in FIG. 6C, lock block 324 may be operatively coupled to motor 322, such that upon actuation of motor 322, lock block 324 may be configured to move horizontally between a first position where lock block 324 is not engaged with lock portion 66 of connector C4, and a second position where lock block 324 is engaged with lock portion 66 of connector C4, to thereby maintain the angular orientation of lock portion 66 relative to slot 312. For example, the interior of lock block 324 may include a threaded surface configured to envelop the threaded axel of motor 322, such that upon rotation of the threaded axel via motor 322, lock block 324 may slide along the threaded axel within channel 316 between the first and second positions. Accordingly, channel 316 may be positioned along slot 312 such that channel 316 and, accordingly, lock block 324, are aligned with lock portion 66 of connector C4 when connector C4 is disposed within slot 312.

In addition, as shown in FIG. 6D, slot 312 further may include opening 318 sized and shaped to permit at least a portion of rotatable drive gear 329 to extend therethrough for engagement with retainer gear 370. As shown in FIG. 6C, drive gear 329 may be operatively coupled to motor 328, such that upon actuation of motor 328, drive gear 329 may be configured to rotate about its longitudinal axis, at least partially through opening 318 of slot 312. Accordingly, upon rotation of drive gear 329 via motor 328, the engagement between drive gear 329 and retaining gear 370 causes rotational movement of retainer gear 370, and accordingly, connector C4 relative to slot 312. Thus, rotation of drive gear 329 in a first direction (e.g., clockwise) causes rotation of retainer gear 370 in a second direction (e.g., counter-clockwise) opposite the first direction, and rotation of drive gear 329 in the second direction (e.g., counter-clockwise) causes rotation of retainer gear 370 in the first direction (e.g., clockwise) opposite the second direction. In addition, as shown in FIG. 6B, fixed platform 310 may include cap hook 320 protruding upwardly from the upper surface of fixed platform 310, and configured to be received within the pull ring of the connector configured to be coupled to movable platform 330, e.g., pull ring 52 of removable cap 50 of connector C3.

As shown in FIG. 6B, the upper surface of fixed platform 310 may include channel 384 sized and shaped to permit lock cover 382 to pass therethrough for engagement with retainer gear 370. As shown in FIG. 6D, lock cover 382 may be operatively coupled to motor 380, such that upon actuation of motor 380, lock cover 382 may be configured to move horizontally between a first position where lock cover 382 is not engaged with retainer gear 370, and a second position where lock cover 382 is engaged with retainer gear 370, to thereby prevent vertical movement of retainer gear 370 relative to slot 312 and, accordingly, connector C4. For example, the interior of lock cover 382 may include a threaded surface configured to envelop the threaded axel of motor 380, such that upon rotation of the threaded axel via motor 380, lock cover 382 may slide along the threaded axel within channel 384 between the first and second positions. Accordingly, channel 384 may be positioned along slot 312 such that channel 384 and, accordingly, cover lock 382, are aligned with retainer gear 370 when connector C4 is disposed within slot 312.

Referring now to FIGS. 6E and 6F, movable platform 330 is provided. As shown in FIG. 6E, the upper surface of movable platform 330 may include slot 332 configured to releasably receive connector C3 therein. Slot 332 may include cavity 334 having plurality of grooves 335 having a geometry that corresponds with the geometry of ridges 56 of connector C3, such that ridges 56 may engage with plurality of grooves 335 when connector C3 in inserted within slot 332, to thereby prevent rotation of connector C3 relative to slot 332. Moreover, the proximal wall defining cavity 334 may abut against the distal side of lip 54 when connector C3 is disposed within slot 332 to thereby prevent proximal movement of connector C3 relative to slot 332.

As shown in FIG. 6E, slot 332 further may include channel 336 sized and shaped to permit clamp block 344 to pass therethrough for engagement with the tubing of cycler patient line ML4. As shown in FIG. 6E, clamp block 344 may be operatively coupled to motor 342, such that upon actuation of motor 342, clamp block 344 may be configured to move horizontally between a first position where clamp block 344 is not engaged with the tubing of cycler patient line ML4, and a second position where clamp block 344 is engaged with the tubing of cycler patient line ML4, to thereby clamp the tubing and prevent fluid flow between cycler patient line ML4 and connector C3. For example, the interior of clamp block 344 may include a threaded surface configured to envelop the threaded axel of motor 342, such that upon rotation of the threaded axel via motor 342, clamp block 344 may slide along the threaded axel within channel 336 between the first and second positions. Accordingly, channel 336 may be positioned along slot 332 such that channel 336 and, accordingly, clamp block 344, are aligned with tubing of cycler patient line ML4 when connector C3 is disposed within slot 332.

In addition, moveable platform 330 may include holder 338 distal to slot 332 and having a groove sized and shaped to receive the tubing of cycler patient line ML4. While FIG. 6E illustrates holder 338 spaced apart from slot 332, in some embodiments, holder 338 may be integrally formed with slot 332. Additionally, or alternatively, holder 338 may also include a sensor configured to detect chemical levels of the fluid such as glucose. Further, moveable platform 330 may include cap holder 340 having a cavity having a geometry sized and shaped to receive gripping portions 62 of cap portion 60 of connector C4 in a manner such that, when gripping portions 62 is received within cap holder 340, rotation between cap portion 60 and cap holder 340 is inhibited. As shown in FIG. 6F, movable platform 330 may be configured to be movable relative to platform 311 and, accordingly, fixed platform 310, e.g., along the horizontal X-axis and horizontal Y-axis, via first and second slide tables 350a, 350b. First and second slide tables 350a, 350b may be constructed similar to first and second slide tables 250a, 250b of case 200.

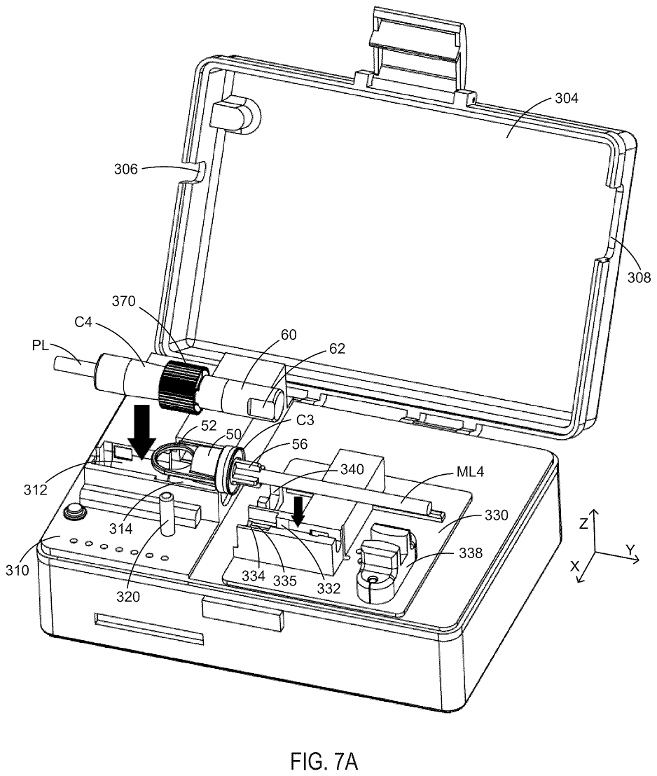

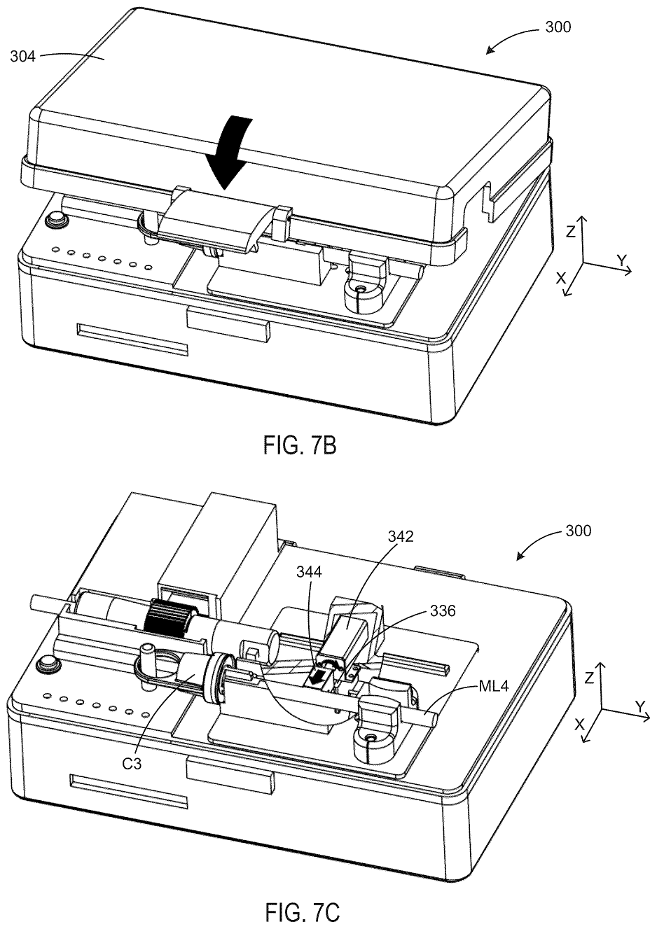

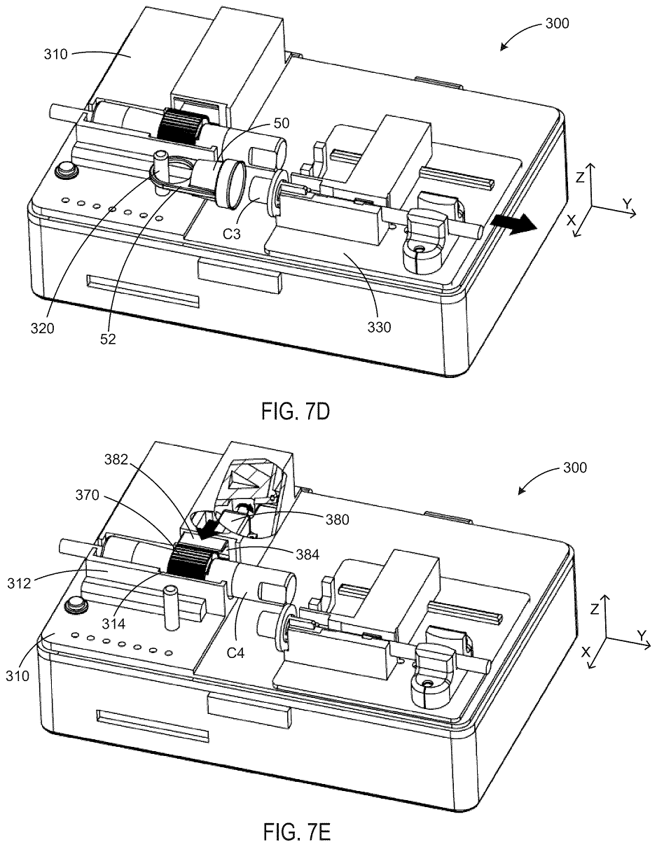

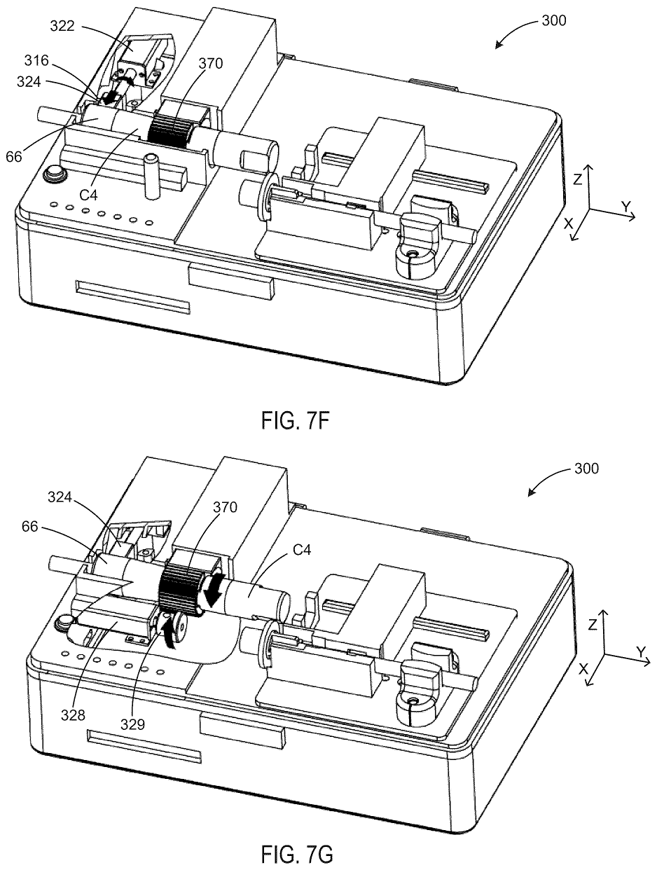

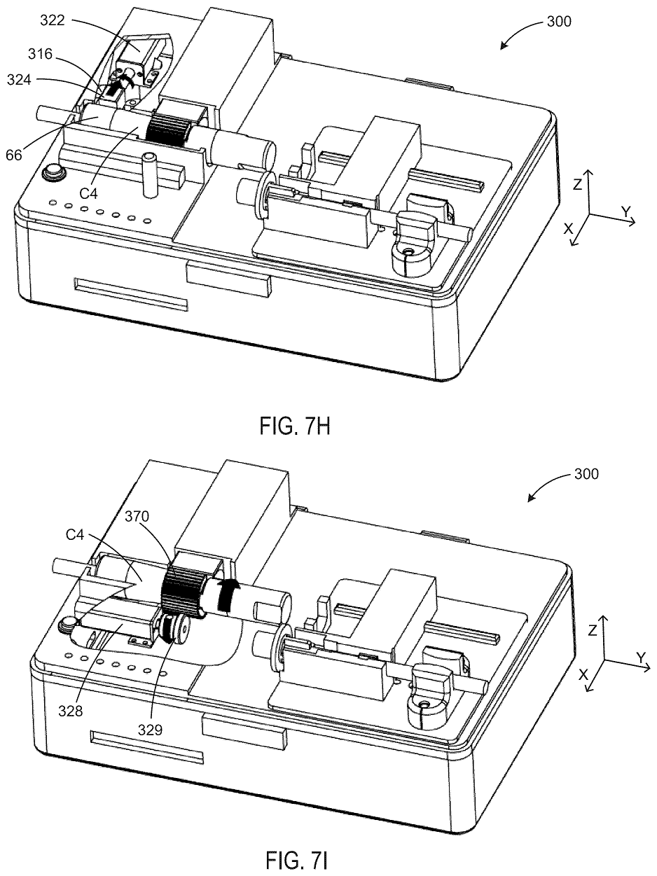

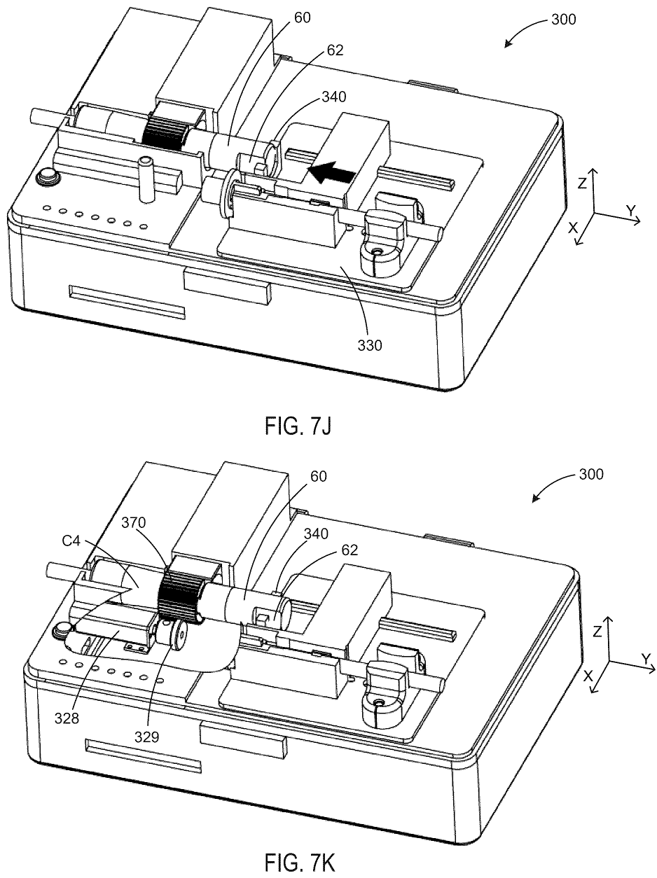

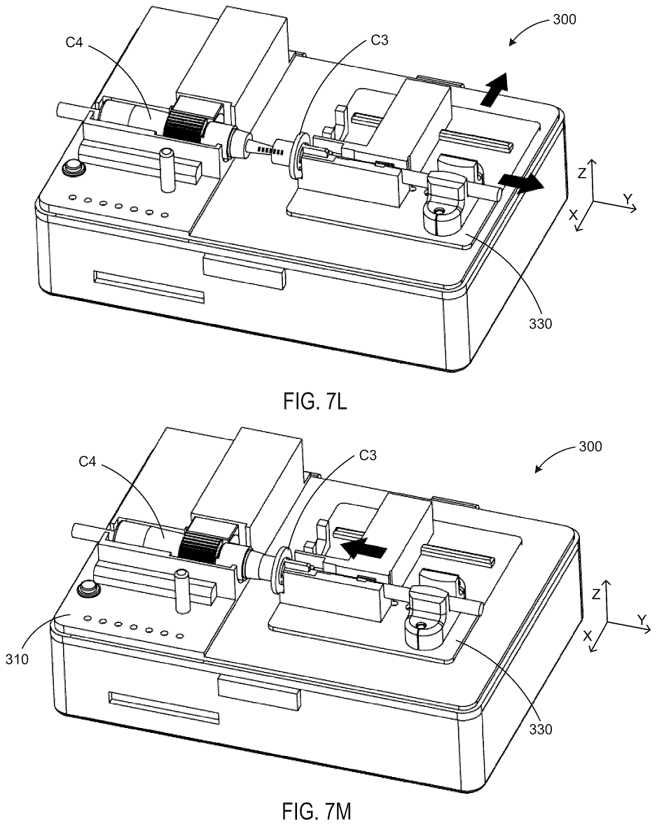

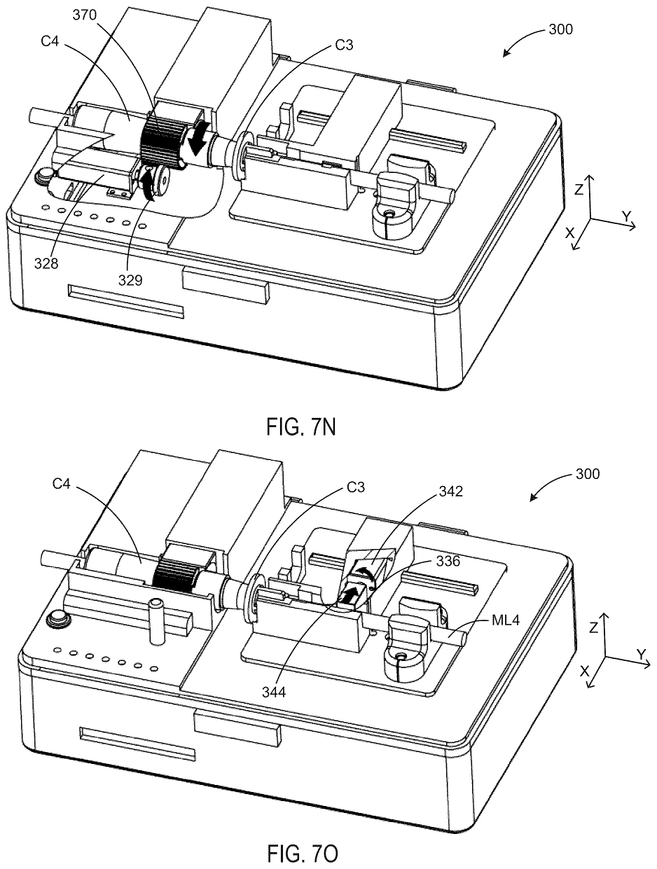

Referring now to FIGS. 7A to 7O, exemplary method steps for automated connection of connector type C3 and connector type C4 via case 300 to thereby fluidically couple transfer set PL and cycler patient line ML4 is provided. As shown in FIG. 7A, the user may insert connector C3 into slot 332 of movable platform 330 such that ridges 56 are disposed within grooves 335 within cavity 334 of slot 332. Moreover, the user may insert a portion of the tubing of cycler patient line ML4 within the channel of holder 338, and position another portion of the tubing of cycler patient line ML4 within window 308 (e.g., when lid 304 is closed). Next, the user may insert connector C4 into slot 312 of fixed platform 310 such that retainer gear 370 is disposed within cavity 314 and gripping portions 62 of cap portion 60 is disposed within cap holder 340 of movable platform 330. In addition, the user may position the tubing of transfer set PL within window 306 (e.g., when lid 304 is closed). The user may then wrap pull ring 52 of cap 50 over hook 320, such that hook 320 is received within and extend upwardly through pull ring 52. The user may then close lid 304 of case 300, as shown in FIG. 7B. No further actions by the user are required for the automated connection of connector C3 and connector C4 and, accordingly, cycler patient line ML4 and transfer set PL via case 300 beyond this stage.

Accordingly, upon actuation of case 300, e.g., via a controller of automated dialysis line connection system 100, as shown in FIG. 7C, motor 342 may be automatically actuated in a first direction to cause clamp block 344 to slide from its first disengaged position along the horizontal X-axis within channel 336 to its second engaged position where it clamps the tubing of cycler patient line ML4 to thereby prevent fluid flow between cycler patient line ML4 and connector C3. Next, as shown in FIG. 7D, movable platform 330 may be automatically moved in a first direction from a first position along the horizontal Y-axis to a second position along the horizontal Y-axis away from fixed platform 310 (e.g., via slide table 350a), such that the engagement between hook 320 and pull ring 52 causes hook 320 to apply a force against pull ring 52 as movable platform 330 and, accordingly, connector C3 moves away from fixed platform 310, thereby pulling cap 50 off of connector C3.

Next, as shown in FIG. 7E, motor 380 may be automatically actuated in a first direction to cause lock cover 382 to slide from its first disengaged position along the horizontal X-axis within channel 384 to its second engaged position where it engages retainer gear 370 such that retainer gear 370 and, accordingly, connector C4, is prevented from vertical Z-axis movement relative to slot 312, such that connector C4 is not rotatable relative to lock block 324 (not shown), e.g., upon rotation of retainer gear 370.

Next, as shown in FIG. 7F, motor 322 may be automatically actuated in a first direction to cause lock block 324 to slide from its first disengaged position along the horizonal X-axis within channel 316 to its second engaged position where it engages lock portion 66 of connector C4 with sufficient friction such that lock portion 66 is not rotatable relative to lock block 324, e.g., upon rotation of retainer gear 370. As shown in FIG. 7G, motor 328 may then be automatically actuated in a first direction to cause drive gear 329 to rotate in the first direction, such that the engagement between drive gear 329 and retainer gear 370 causes retainer gear 370 and, accordingly, connector C4 to rotate in a second direction opposite the first direction of rotation of drive gear 329 while lock portion 66 remains stationary due to its engagement with lock block 324, to thereby cause lock portion 66 to transition from its locked state to its unlocked state where the twist lock of connector C4 permits fluid flow therethrough. As shown in FIG. 7H, motor 322 may then be automatically actuated in a second direction to cause lock block 324 to slide from its second engaged position along the horizontal X-axis within channel 316 to its first disengaged position where it disengages lock portion 66 of connector C4.

As shown in FIG. 7I, motor 328 may then be automatically actuated in the second direction to cause drive gear 329 to rotate in the second direction, such that the engagement between drive gear 329 and retainer gear 370 causes retainer gear 370 and, accordingly, connector C4 to rotate in the first direction opposite the second direction of rotation of drive gear 329. Movable platform 330 may then be automatically moved in the second direction from the second position along the horizontal Y-axis back to the first position along the horizontal Y-axis (e.g., via slide table 350a), as shown in FIG. 7J, such that gripping portions 62 of cap portion 60 is re-inserted into cap holder 340 of movable platform 330, as shown in FIG. 7K. As shown in FIG. 7K, motor 328 may then be automatically actuated again in the second direction to cause drive gear 329 to rotate in the second direction, such that the engagement between drive gear 329 and retainer gear 370 causes retainer gear 370 and, accordingly, connector C4 to rotate in the first direction opposite the second direction of rotation of drive gear 329, while cap portion 60 remains stationary due to the engagement between gripping portions 62 and cap holder 340, thereby unscrewing and removing cap portion 60 from connector C4.

Next, as shown in FIG. 7L, with the respective caps removed, movable platform 330 may then be automatically moved in the first direction from the first position along the horizontal Y-axis back to the second position along the horizontal Y-axis (e.g., via slide table 350a), and then automatically moved in a first direction from a first position along the horizontal X-axis to a second position along the horizontal X-axis (e.g., via slide table 350b), to thereby align connector C3 coaxially with connector C4. As shown in FIG. 7M, movable platform 330 may then be automatically moved in the second direction from the second position along the horizontal Y-axis to a third position along the horizontal Y-axis toward fixed platform 310 (e.g., via slide table 350a) such that connector C3 is engaged with connector C4. In some embodiments, the third position along the horizontal Y-axis may be the same as the first position along the horizontal Y-axis.

Next, as shown in FIG. 7N, motor 328 may then be automatically actuated in the first direction to cause drive gear 329 to rotate in the first direction, such that the engagement between drive gear 329 and retainer gear 370 causes retainer gear 370 and, accordingly, connector C4 to rotate in the second direction opposite the first direction of rotation of drive gear 329, thereby tightly connecting connector C4 to connector C3, e.g., via a screw type engagement between connector C4 and connector C3. In some embodiments, connector type C3 and connector type C4 may both include double-start threads, such that each may only need to rotate 180 degrees for full attachment and/or removal. Upon connection of connector C4 to connector C3, as shown in FIG. 7O, motor 342 may then be automatically actuated in a second direction to cause clamp block 344 to slide from its second engaged position along the horizonal X-axis within channel 336 to its first disengaged position where it unclamps the tubing of cycler patient line ML4 to thereby permit fluid flow between cycler patient line ML4 and connector C3.

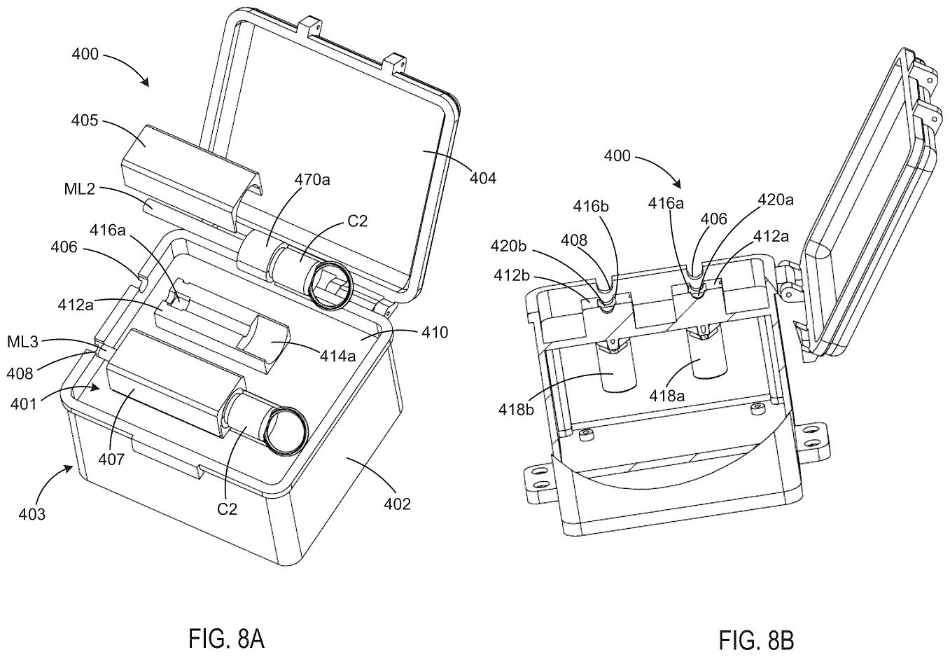

Referring now to FIGS. 8A and 8B, an exemplary case for automated clamping of unused dialysis lines is provided. As described above, dialysis circuit 20 may include a plurality of cycler solution lines, e.g., cycler solution lines ML1, ML2, ML3, for fluidic connection with one or more dialysis bags. When only one or two dialysis bags are used, e.g., fluidically coupled to cycler solution line ML1, any unused cycler solution lines, e.g., cycler solution lines ML2 and/or ML3, may clamped to prevent fluid flow therethrough, at least temporarily, until they are required for fluidically coupling one or more dialysis bags. Accordingly, shown in FIG. 8A, case 400 may be selected for automatically clamping unused cycler solution lines, e.g., cycler solution lines ML2, ML3, having connector type C2 to thereby prevent fluid flow therethrough. For illustrative purposes, FIG. 8A illustrates cycler solution lines ML2 having connector type C2; however, any unused dialysis lines having connector type C2 may be automatically clamped via case 400.

As shown in FIGS. 8A and 8B, case 400 may include housing 402, and lid 404 configured to enclose housing 402. Housing 402 may include first window 406 and second window 408, e.g., along the upper edge of housing 402, each sized and shaped to receive the tubing of a respective dialysis line therein in a manner that does not disrupt fluid flow therethrough, such that upon closure of lid 404, the respective dialysis lines will be maintained within first window 406 and second window 408. As will be understood by a person having ordinary skill in the art, while FIGS. 8A and 8B illustrate first window 406 and second window 408 disposed on the same side of housing 402, they may be disposed on opposite sides of housing 402, depending on the position/orientation of the slots for receiving the line connectors, as described in further detail below.

Housing 402 may include horizontally extending fixed platform 410 disposed therein and fixed thereto that divides the interior of housing 402 upper chamber 401 and lower chamber 403, to thereby block pathogens from the lower chamber from entering the upper chamber which holds the dialysis lines. For example, the connectors and associated dialysis lines may be positioned within upper chamber 401, while the moving mechanisms and electronic controls of case 200 may be disposed within lower chamber 403. The upper surface of fixed platform 410 may include one or more slots, e.g., slot 412a, 412b, each configured to releasably receive connector type C2 therein, and further may include one or more covers, e.g., cover 405, 407, configured to be removably disposed over slots 412a, 412b, respectively, to thereby maintain and protect the line connectors disposed therein during operation of case 200. Slot 412a and slot 412b may be structurally identical to each other, and may be constructed similar to slot 232 of case 200, except without an opening sized and shaped to permit at least a portion of an angled, rotatable drive gear to extend therethrough. For example, slots 412a, 412b may include cavities 414a, 414b, respectively, each sized and shaped to receive lip 44 of connector type C2 and retainer gears 470a, 470b, therein in a manner that prevents axial movement of the line connectors relative to slots 412a, 412b, and accordingly, fixed platform 410. For example, the proximal wall of cavities 414a, 414b may abut against the proximal side of lips 44 of the respective line connector to thereby prevent proximal movement of the line connector relative to slots 412a, 412b. Retainer gears 470a, 470b may be constructed similar to retainer gear 270, as described above.

In addition, slots 412a, 412b further may include channels 416a, 416b sized and shaped to permit clamp blocks 420a, 420b to pass therethrough for engagement with the tubings of the respective cycler solution lines ML2, ML3. As shown in FIG. 8B, clamp blocks 420a, 420b may be operatively coupled to motors 416a, 416b, respectively, such that upon actuation of motors 416a, 416b, clamp blocks 420a, 420b may be configured to move vertically between a first position where clamp blocks 420a, 420b is not engaged with the respective tubing of the cycler solution line, and a second position where clamp blocks 420a, 420b is engaged with the respective tubing of the cycler solution line, to thereby clamp the respective tubing and prevent fluid flow between cycler solution lines ML2, ML3 and the respective connectors C2. For example, the interior of clamp blocks 420a, 420b may include a threaded surface configured to envelop the respective threaded axel of motors 416a, 416b, such that upon rotation of the threaded axels via motors 416a, 416b, clamp blocks 420a, 420b may slide along the respective threaded axel within channels 416a, 416b, respectively, between the first and second positions. Accordingly, channels 416a, 416b may be positioned along slots 412a, 412b such that channels 416a, 416b, and accordingly clamp blocks 420a, 420b, are aligned with tubings of cycler solution lines ML2, ML3 when the line connectors are disposed within slots 412a, 412b. As will be understood by a person having ordinary skill in the art, while FIGS. 8A and 8B illustrate identical slots 412a, 412b, case 400 may include different type slots for clamping dialysis lines having different connector types. For example, instead of slot 412a and/or slot 412b, case 400 may include one or more slots configured for receiving any one of connector types C1, C3, or C4, as described above, and including a channel sized and shaped to permit the clamp block to pass therethrough, to thereby automatically clamp the dialysis line connected thereto.

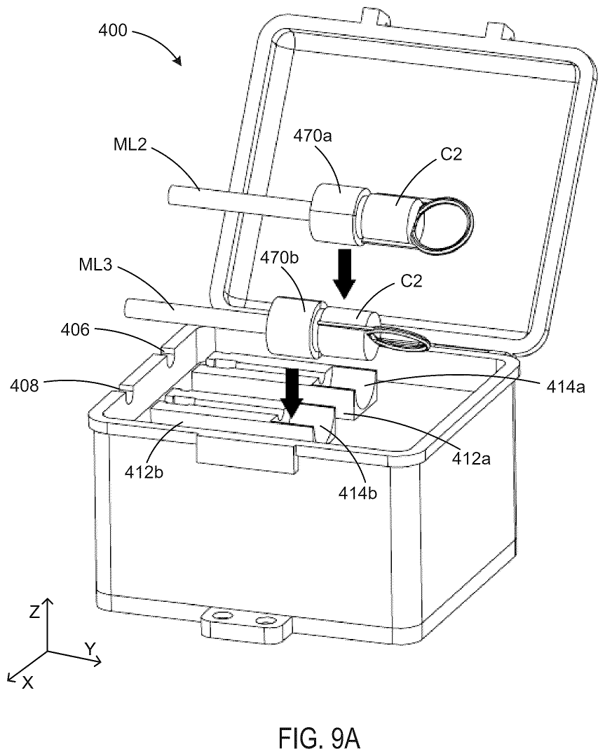

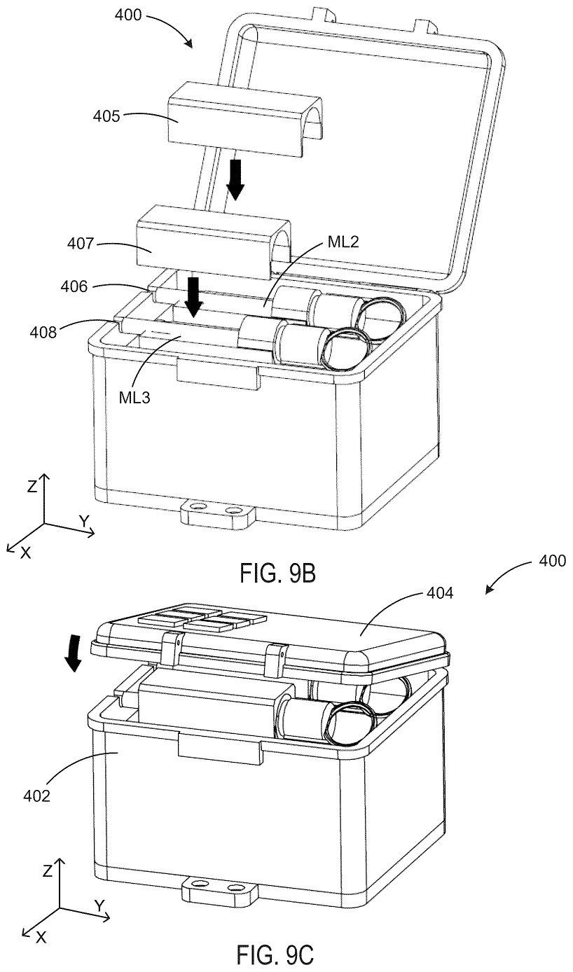

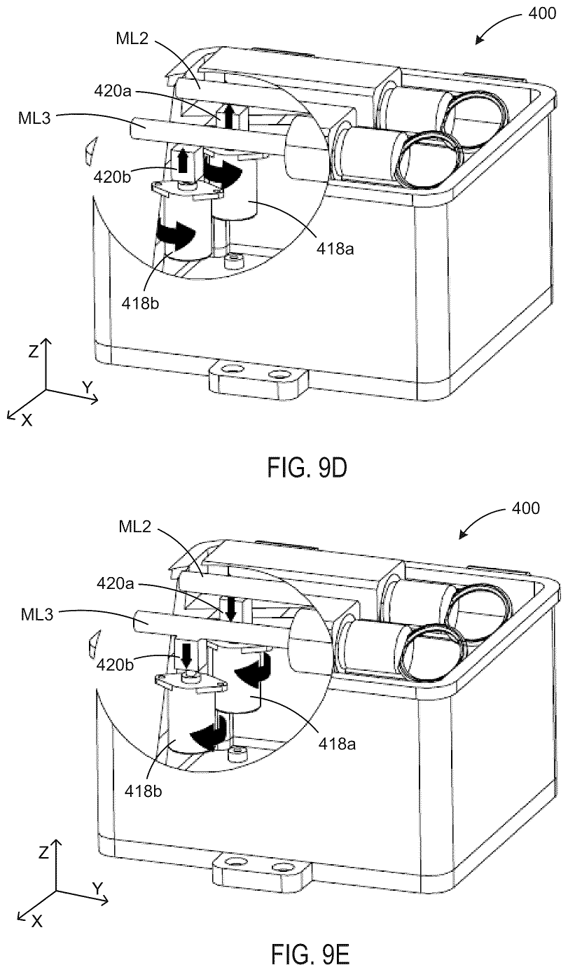

Referring now to FIGS. 9A to 9E, exemplary method steps for automated clamping of one or more unused dialysis lines having connector type C2 via case 400 is provided. As shown in FIG. 9A, the user may insert connector C2 of cycler solution line ML2 into slot 412a of fixed platform 410 and connector C2 of cycler solution line ML3 into slot 412b of fixed platform 410, such that retainer gears 470a, 470b are disposed within cavities 414a, 414b, respectively. As shown in FIG. 9B, the user may position covers 405, 407 over the line connectors within slots 412a, 412b, and further may position the tubing of cycler solution line ML2 within window 406 and the tubing of cycler solution line ML3 within window 408. The user may then close lid 404 of case 400, as shown in FIG. 9C. No further actions by the user are required for the automated clamping of cycler solution lines ML2, ML3 via case 400 beyond this stage.

As shown in FIG. 9D, motors 416a, 416b may be automatically actuated in a first direction to cause clamp blocks 420a, 420b to slide from their first disengaged position along the vertical Z-axis within channels 416a, 416b to their second engaged position where they clamp the respective tubings of cycler solution lines ML2, ML3 to thereby prevent fluid flow between cycler solution lines ML2, ML3 and the respective connector C2. When cycler solution lines ML2, ML3 are ready for use, e.g., coupling with a respective bag line, as shown in FIG. 9E, motors 416a, 416b may be automatically actuated in a second direction to cause clamp blocks 420a, 420b to slide from their second engaged position along the vertical Z-axis within channels 416a, 416b to their first disengaged position where they unclamp the respective tubings of cycler solution lines ML2, ML3.

As will be understood by a person having ordinary skill in the art, each of the cases described herein may be configured to fluidically connect any two of the connector types described herein, based on the specific connector type and the type of dialysis line coupled thereto. For example, for dialysis lines that may require clamping, the respective slot may include a channel for slidably receiving a clamp block therethrough, for dialysis lines/connector types that may require rotation, e.g., for unlocking, unscrewing, screw type coupling, etc., the respective slots may include an opening for receiving the angled drive gear therethrough, for dialysis lines that may require unlocking, the respective slot may include a channel for slidably receiving a lock block therethrough, for connector types that have removable caps with a pull ring, the case may include a hook for engaging the pull ring and pulling off the cap, for connector types that have a cap having a gripping portion, the case may include a cap holder having the geometry for tightly receiving the gripping portion of the cap, etc. Accordingly, case 500 of FIG. 1 may be configured to fluidically couple cycler drain line ML5 having any connector type C1-C4 with drain bag line DL having any connector type C1-C4.

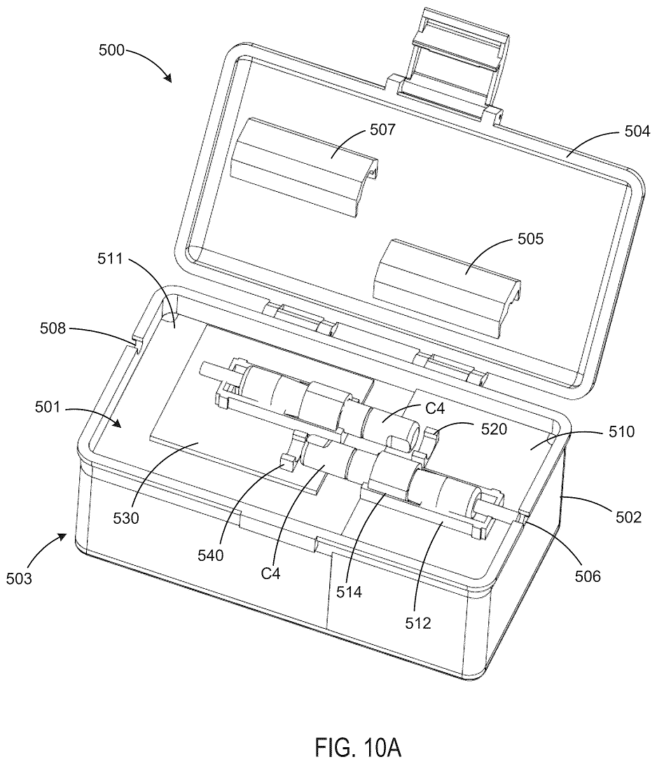

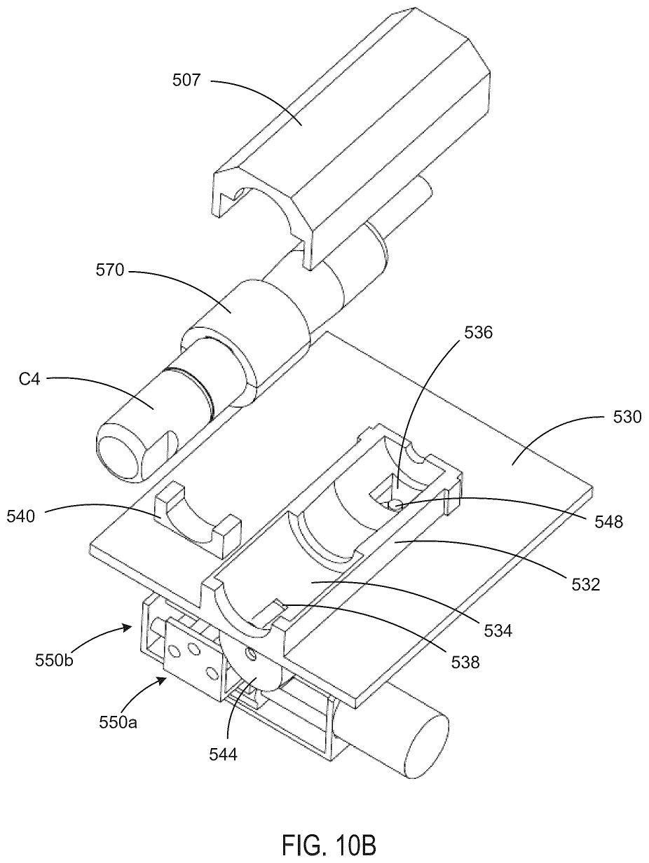

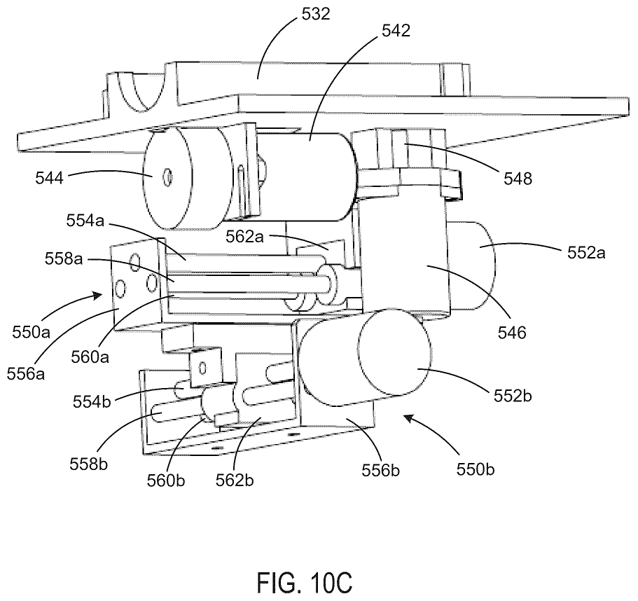

For example, referring now to FIGS. 10A to 10C, an exemplary case for automated connection of two dialysis lines, each having connector type C4 is provided. For example, one of the dialysis lines may be a cycler drain line, e.g., cycler drain line ML5, and the other dialysis line may a drain bag line, e.g., drain bag line DL, coupled to a drain bag, e.g., drain bag 112. Accordingly, case 500 may be selected for automatically coupling connector type C4 to connector type C4 to thereby fluidically couple the cycler drain line with the drain bag line, and accordingly, cycler 20 with drain bag 112 to permit fluid flow therebetween. As shown in FIG. 10A, case 500 may include housing 502, and lid 504 configured to enclose housing 502. Housing 502 may include first window 506 and second window 508, e.g., along the upper edge of housing 502, each sized and shaped to receive the tubing of a respective dialysis line therein in a manner that does not disrupt fluid flow therethrough, such that upon closure of lid 504, the respective dialysis lines will be maintained within first window 506 and second window 508.

Housing 502 may include horizontally extending platform 511 disposed therein that divides the interior of housing 502 upper chamber 501 and lower chamber 503. For example, the connectors and associated dialysis lines may be positioned within upper chamber 501, while the moving mechanisms and electronic controls of case 200 may be disposed within lower chamber 503. Platform 511 may include fixed platform 510 that is fixed relative to housing 502, and an opening sized and shaped to receive at least a portion of movable platform 530 therethrough, such that movable platform 530 may be actuated to move relative to platform 511, and accordingly, fixed platform 510. The upper surface of fixed platform 510 may include slot 512 configured to releasably receive connector type C4 therein, and further may include cover 505 configured to be removably disposed over slot 512 to thereby maintain and protect connector type C4 within slot 512 during operation of case 500. Slot 512 may include cavity 514 sized and shaped to receive lip 64 of connector type C4 and retainer gear 570 therein in a manner that prevents axial movement of connector type C4 relative to slot 512, and accordingly, fixed platform 510. For example, the proximal wall of cavity 514 may abut against the proximal side of lip 64 to thereby prevent proximal movement of connector type C4 relative to slot 512. Retainer gears 570 may be constructed similar to retainer gear 370, as described above.

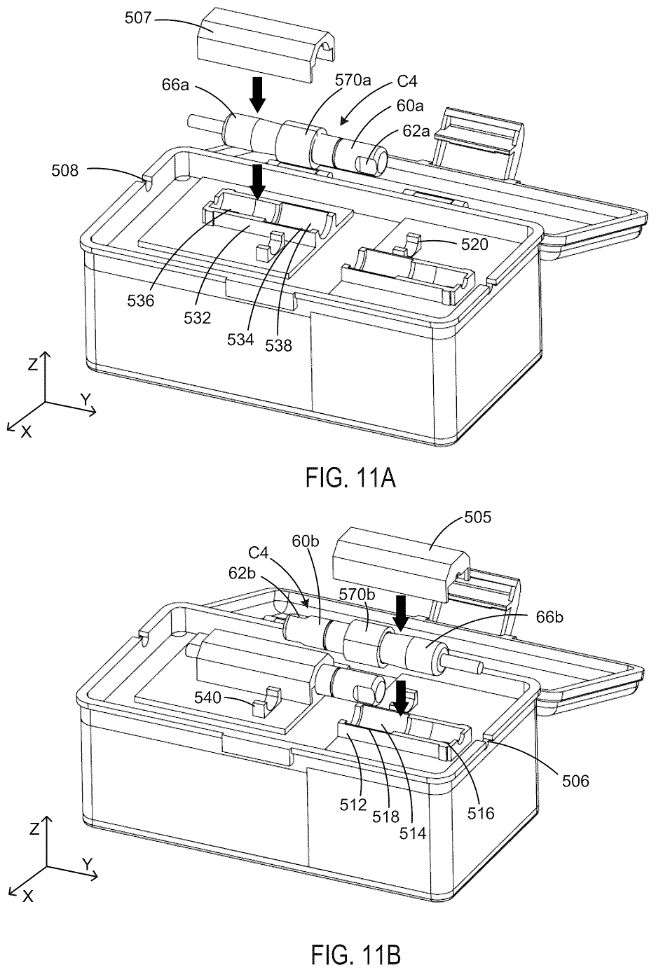

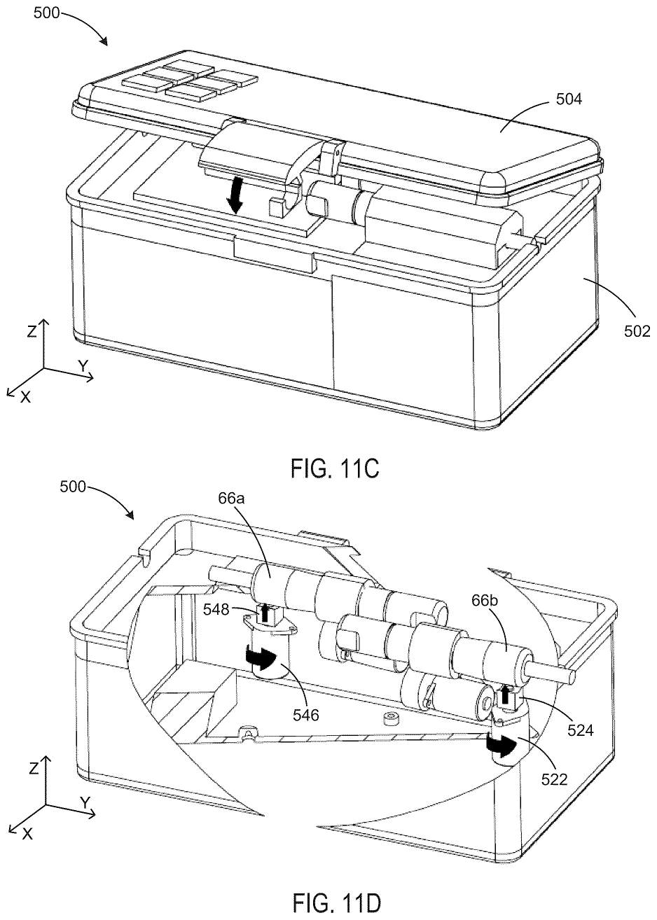

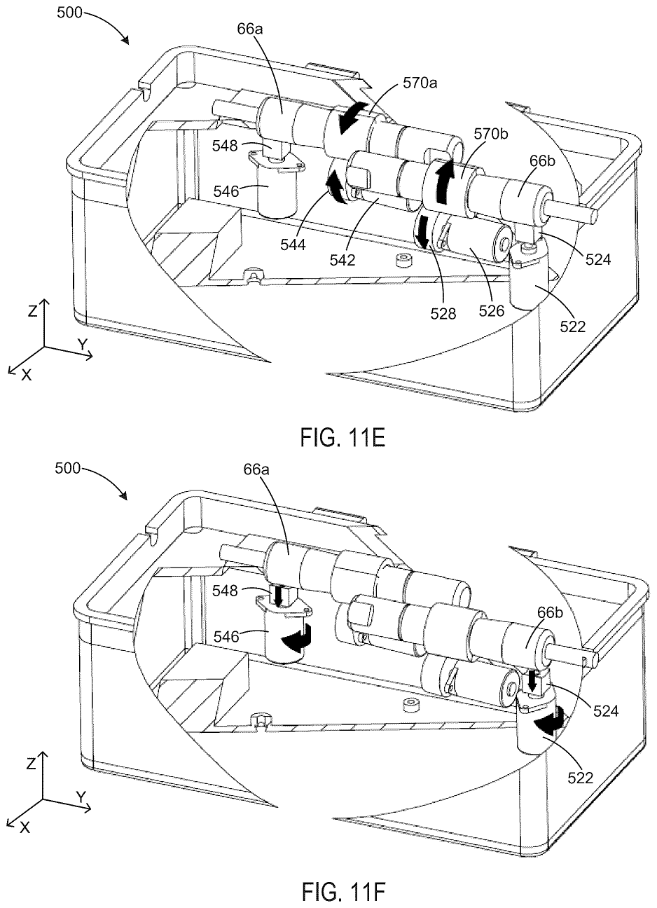

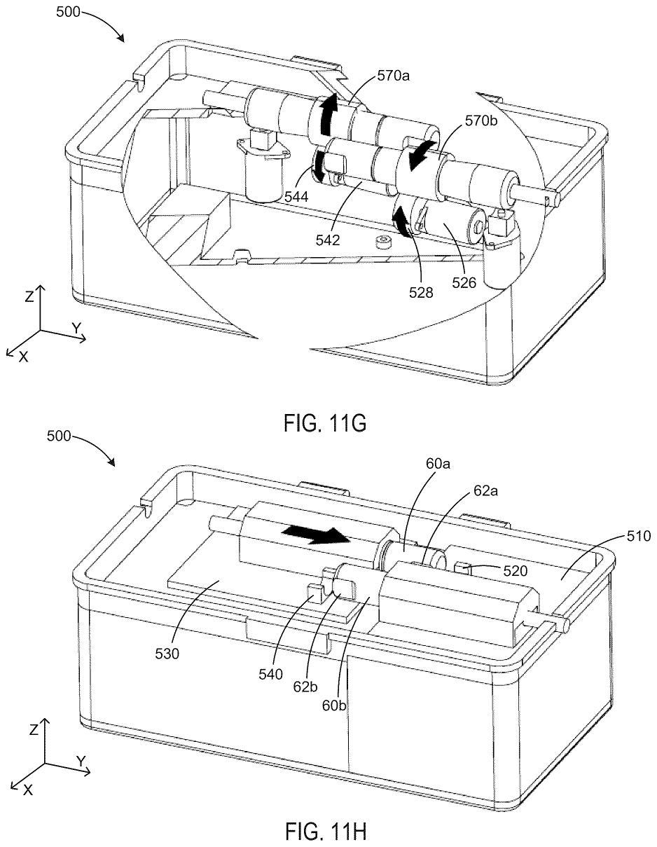

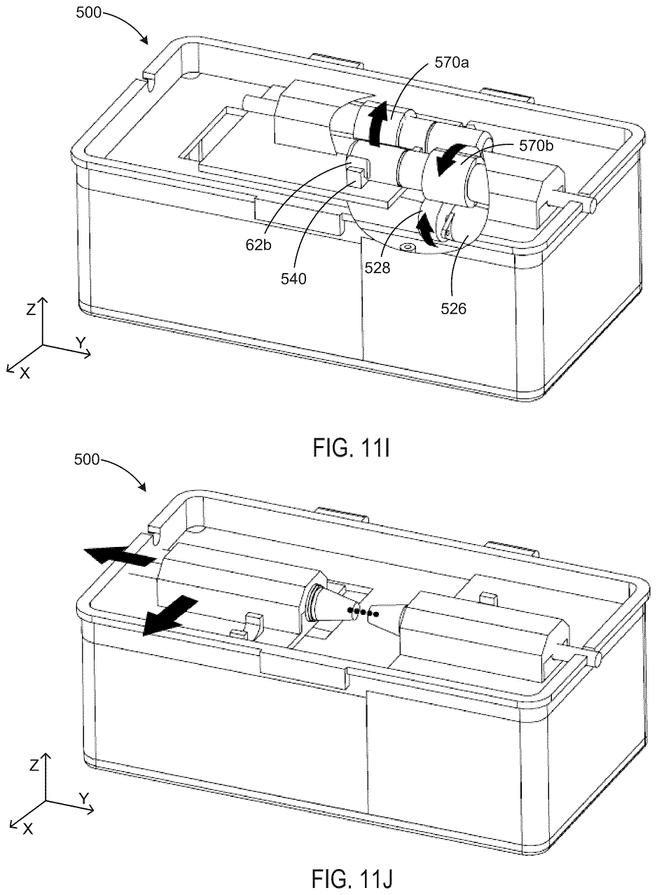

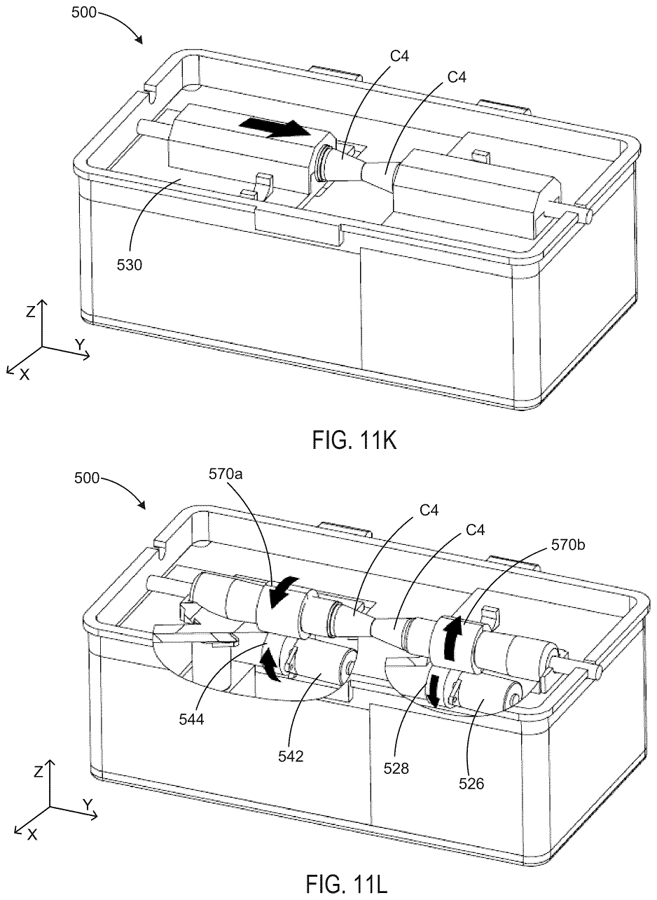

As shown in FIGS. 11B and 11D, slot 512 further may include channel 516 sized and shaped to permit lock block 524 to pass therethrough for engagement with lock portion 66b of connector type C4. As shown in FIG. 11D, lock block 524 may be operatively coupled to motor 522, such that upon actuation of motor 522, lock block 524 may be configured to move vertically between a first position where lock block 524 is not engaged with lock portion 66b of connector type C4, and a second position where lock block 524 is engaged with lock portion 66b of connector type C4, to thereby maintain the angular orientation of lock portion 66b relative to slot 512. For example, the interior of lock block 524 may include a threaded surface configured to envelop the threaded axel of motor 522, such that upon rotation of the threaded axel via motor 522, lock block 524 may slide along the threaded axel within channel 516 between the first and second positions. Accordingly, channel 516 may be positioned along slot 512 such that channel 516, and accordingly lock block 524, are aligned with lock portion 66b of connector type C4 when connector type C4 is disposed within slot 512.

In addition, as shown in FIGS. 11B and 11E, slot 512 further may include opening 518 sized and shaped to permit at least a portion of angled, rotatable drive gear 528 to extend therethrough for engagement with retainer gear 570b. As shown in FIG. 11E, drive gear 528 may be operatively coupled to motor 526, such that upon actuation of motor 526, drive gear 528 may be configured to rotate about its longitudinal axis, at least partially through opening 518 of slot 512. Like motor 328 and drive gear 329 of case 300, motor 526 and drive gear 528 may be coupled to the bottom surface of fixed platform 510 at angle relative to slot 512, such that drive gear 528 contacts retainer gear 570b through opening 518 at an incline angle. Accordingly, upon rotation of drive gear 528 via motor 526, the engagement between drive gear 528 and retaining gear 570b causes rotational and linear movement of retainer gear 570b, and accordingly, connector type C4 relative to slot 512. Thus, rotation of drive gear 528 in a first direction (e.g., clockwise) causes rotation of retainer gear 570b in a second direction (e.g., counter-clockwise) opposite the first direction and linear movement retainer gear 570b in a distal direction relative to slot 512, and rotation of drive gear 528 in the second direction (e.g., counter-clockwise) causes rotation of retainer gear 570b in the first direction (e.g., clockwise) opposite the second direction and linear movement retainer gear 570b in a proximal direction relative to slot 512. In addition, fixed platform 510 may include cap holder 520 having a cavity having a geometry sized and shaped to receive gripping portions 62b of cap portion 60b of connector type C4 in a manner such that, when gripping portions 62b is received within cap holder 520, rotation between cap portion 60b and cap holder 520 is inhibited.

Referring now to FIGS. 10B and 10C, movable platform 530 is provided. As shown in FIGS. 10B and 10C, the upper surface of movable platform 530 may include slot 532 configured to releasably receive connector type C4 therein, and further may include cover 507 configured to be removably disposed over slot 532 to thereby maintain and protect connector type C4 within slot 532 during operation of case 500. Slot 532 may be structurally identical to 512. For example, cavity 534 sized and shaped to receive lip 64a of connector type C4 and retainer gear 570a corresponds with cavity 514, channel 536 sized and shaped to permit lock block 548 operatively coupled to motor 546 to pass therethrough for engagement with lock portion 66a of connector type C4 corresponds with channel 516, and opening 538 sized and shaped to permit at least a portion of angled, rotatable drive gear 544 operatively coupled to motor 542 to extend therethrough for engagement with retainer gear 570a corresponds with opening 518. In addition, movable platform 530 may include cap holder 540 having a cavity having a geometry sized and shaped to receive gripping portions 62a of cap portion 60a of connector type C4 in a manner such that, when gripping portions 62a is received within cap holder 540, rotation between cap portion 60a and cap holder 540 is inhibited.

Movable platform 530 may be configured to be movable relative to platform 511, and accordingly, fixed platform 510, e.g., along the horizontal X-axis and horizontal Y-axis, via first and second slide tables 550a, 550b. First and second slide tables 550a, 550b may be constructed similar to first and second slide tables 250a, 250b of case 200. For example, slide tables 550a, 550b having frames 556a, 556b, support rods 558a, 560a, 558b, 560b, motors 552a, 552b, threaded axels 554a, 554b, and slide blocks 562a, 562b correspond with slide tables 250a, 250b having frames 256a, 256b, support rods 258a, 260a, 258b, 260b, motors 252a, 252b, threaded axels 254a, 254b, and slide blocks 262a, 262b.

Referring now to FIGS. 11A to 11L, exemplary method steps for automated connection of two dialysis lines, e.g., a cycler drain line and a drain bag line, each having connector type C4, is provided. As shown in FIG. 11A, the user may insert first connector C4 into slot 532 of movable platform 530 such that retainer gear 570a is disposed within cavity 534 and gripping portions 62a of cap portion 60a is aligned with cap holder 520 of fixed platform 510. In addition, the user may position the tubing of the dialysis line within window 508. As shown in FIG. 11B, the user may insert second connector C4 into slot 512 of fixed platform 510 such that retainer gear 570b is disposed within cavity 514 and gripping portions 62b of cap portion 60b is aligned with cap holder 540 of movable platform 530. In addition, the user may position the tubing of the dialysis line within window 506. The user may then close lid 504 of case 500, as shown in FIG. 11C. No further actions by the user are required for the automated connection of the two connectors C4, and accordingly via case 500 beyond this stage.

As shown in FIG. 11D, motor 522 may be automatically actuated in a first direction to cause lock block 524 to slide from its first disengaged position along the vertical Z-axis within channel 516 to its second engaged position where it engages lock portion 66b of second connector C4 with sufficient friction such that lock portion 66b is not rotatable relative to lock block 524, e.g., upon rotation of retainer gear 570b. In addition, motor 544 may be automatically actuated in a first direction to cause lock block 546 to slide from its first disengaged position along the vertical Z-axis within channel 536 to its second engaged position where it engages lock portion 66a of first connector C4 with sufficient friction such that lock portion 66a is not rotatable relative to lock block 546, e.g., upon rotation of retainer gear 570a. Motors 522 and 544 may be actuated simultaneously.

As shown in FIG. 11E, motor 526 may then be automatically actuated in a first direction to cause drive gear 528 to rotate in the first direction, such that the engagement between drive gear 528 and retainer gear 570b at the incline angle causes retainer gear 570b, and accordingly, second connector C4 to rotate in a second direction opposite the first direction of rotation of drive gear 528 while lock portion 66b remains stationary due to its engagement with lock block 524, while simultaneously moving linearly along the Y-axis away from fixed platform 510, to thereby cause lock portion 66b to transition from its locked state to its unlocked state where the twist lock of second connector C4 permits fluid flow therethrough. In addition, motor 542 may then be automatically actuated in a first direction to cause drive gear 544 to rotate in the first direction, such that the engagement between drive gear 544 and retainer gear 570a at the incline angle causes retainer gear 570a, and accordingly, first connector C4 to rotate in a second direction opposite the first direction of rotation of drive gear 544 while lock portion 66a remains stationary due to its engagement with lock block 548, while simultaneously moving linearly along the Y-axis away toward fixed platform 510, to thereby cause lock portion 66a to transition from its locked state to its unlocked state where the twist lock of first connector C4 permits fluid flow therethrough. Motors 526 and 542 may be actuated simultaneously.

As shown in FIG. 11F, motor 522 may then be automatically actuated in a second direction to cause lock block 524 to slide from its second engaged position along the vertical Z-axis within channel 516 to its first disengaged position where it disengages lock portion 66b of second connector C4, and motor 546 may then be automatically actuated in a second direction to cause lock block 548 to slide from its second engaged position along the vertical Z-axis within channel 536 to its first disengaged position where it disengages lock portion 66a of first connector C4. As shown in FIG. 11G, motor 526 may then be automatically actuated in the second direction to cause drive gear 528 to rotate in the second direction, such that the engagement between drive gear 528 and retainer gear 570b at the incline angle causes retainer gear 570b, and accordingly, second connector C4 to rotate in the first direction opposite the second direction of rotation of drive gear 528 while simultaneously moving linearly along the Y-axis toward fixed platform 510 back to its initial position along the Y-axis. In addition, motor 542 may then be automatically actuated in the second direction to cause drive gear 544 to rotate in the second direction, such that the engagement between drive gear 544 and retainer gear 570a at the incline angle causes retainer gear 570a, and accordingly, first connector C4 to rotate in the first direction opposite the second direction of rotation of drive gear 544 while simultaneously moving linearly along the Y-axis away from fixed platform 510 back to its initial position along the Y-axis. Motors 526 and 542 may be actuated simultaneously.

Movable platform 530 may then be automatically moved in a first direction from a first position along the horizontal Y-axis to a second position along the horizontal Y-axis (e.g., via slide table 550a), as shown in FIG. 11H, such that gripping portions 62a of cap portion 60a is inserted into cap holder 520 of fixed platform 510 and gripping portions 62b of cap portion 60b is inserted into cap holder 540 of movable platform 530, as shown in FIG. 11I. As shown in FIG. 11I, motor 526 may then be automatically actuated again in the second direction to cause drive gear 528 to rotate in the second direction, such that the engagement between drive gear 528 and retainer gear 570b at the incline angle causes retainer gear 570b, and accordingly, second connector C4 to rotate in the first direction opposite the second direction of rotation of drive gear 528 while simultaneously moving linearly along the Y-axis further towards fixed platform 510, and while cap portion 60b remains stationary due to the engagement between gripping portions 62b and cap holder 540, thereby unscrewing and removing cap portion 60b from second connector C4. In addition, motor 542 may then be automatically actuated again in the second direction to cause drive gear 544 to rotate in the second direction, such that the engagement between drive gear 544 and retainer gear 570a at the incline angle causes retainer gear 570a, and accordingly, first connector C4 to rotate in the first direction opposite the second direction of rotation of drive gear 544 while simultaneously moving linearly along the Y-axis further away from fixed platform 510, and while cap portion 60a remains stationary due to the engagement between gripping portions 62a and cap holder 520, thereby unscrewing and removing cap portion 60a from first connector C4. Motors 526 and 542 may be actuated simultaneously.

Next, as shown in FIG. 11J, with the respective caps removed, movable platform 530 may then be automatically moved in a second direction from the second position along the horizontal Y-axis back to the first position along the horizontal Y-axis (e.g., via slide table 550a), and then automatically moved in a first direction from a first position along the horizontal X-axis to a second position along the horizontal X-axis (e.g., via slide table 550b), to thereby align first connector C4 coaxially with second connector C4. As shown in FIG. 11K, movable platform 530 may then be automatically moved in the first direction from the first position along the horizontal Y-axis to a third position along the horizontal Y-axis toward fixed platform 510 (e.g., via slide table 550a) such that first connector C4 is engaged with second connector C4. In some embodiments, the third position along the horizontal Y-axis may be the same as the first position along the horizontal Y-axis.

Next, as shown in FIG. 11L, motor 526 may then be automatically actuated in the first direction to cause drive gear 528 to rotate in the first direction, such that the engagement between drive gear 528 and retainer gear 570b at the incline angle causes retainer gear 570b, and accordingly, second connector C4 to rotate in the second direction opposite the first direction of rotation of drive gear 528, while simultaneously moving linearly along the Y-axis away from fixed platform 510, and motor 542 may then be automatically actuated in the first direction to cause drive gear 544 to rotate in the first direction, such that the engagement between drive gear 544 and retainer gear 570a at the incline angle causes retainer gear 570a, and accordingly, first connector C4 to rotate in the second direction opposite the first direction of rotation of drive gear 544, while simultaneously moving linearly along the Y-axis towards fixed platform 510, thereby tightly connecting first connector C4 to second connector C4, e.g., via a screw type engagement between first connector C4 and second connector C4. In some embodiments, both first and second connectors C4 may include double-start threads, such that each may only need to rotate 180 degrees for full attachment and/or removal.

Moreover, automated dialysis line connection system 100 may include one or more sensors operatively coupled to one or more controllers of the system, and configured to transmit electrical signals therebetween. For example, the case configured for coupling a cycler patient line to the transfer set may include a sensor for measuring the presence/level of liquid within the cycler patient line, e.g., a light sensor, which may be indicative of the completion of the priming phase. For example, the controller may be programmed to, after the cycler patient line is clamped (FIG. 5E), determine the presence of fluid within the cycler patient line following the priming phase prior to initiating the automated cap removal (FIG. 5F).

In addition, the case configured for fluidically coupling a cycler drain line to a drain bag line may include a sensor for detecting the fluid flow velocity in the drain bag line, such that the controller may be programmed to stop the drainage cycle when the flow in the drain bag line falls below a predetermined threshold, e.g., stops. Moreover, the controller may be programmed to calculate how much dialysate still remains in the patient's abdomen, e.g., at least partially based on the fluid flow velocity in the drain bag line, which may be used to provide feedback to the cycler to initiate the next cycle of infusion of dialysate into the patient, without disrupting the patient. For example, with knowledge of the amount of dialysate still remaining in the patient at the end of a first infusion cycle, in the next infusion cycle, the controller may cause the cycler to infuse the intended “normal” amount of dialysate minus the amount of dialysate still remaining in the patient at the end of the first infusion cycle to thereby avoid overfilling the patient's abdominal cavity during the next infusion cycle. Alternatively, without a sensor for detecting the fluid flow velocity in the drain bag line, if the flow in the drain bag line stops before the full drainage of the fluid in the patient's abdomen, the cycler may sound an alarm, requiring the patient to manually intervene to address this issue, which may be disruptive to a sleeping patient.

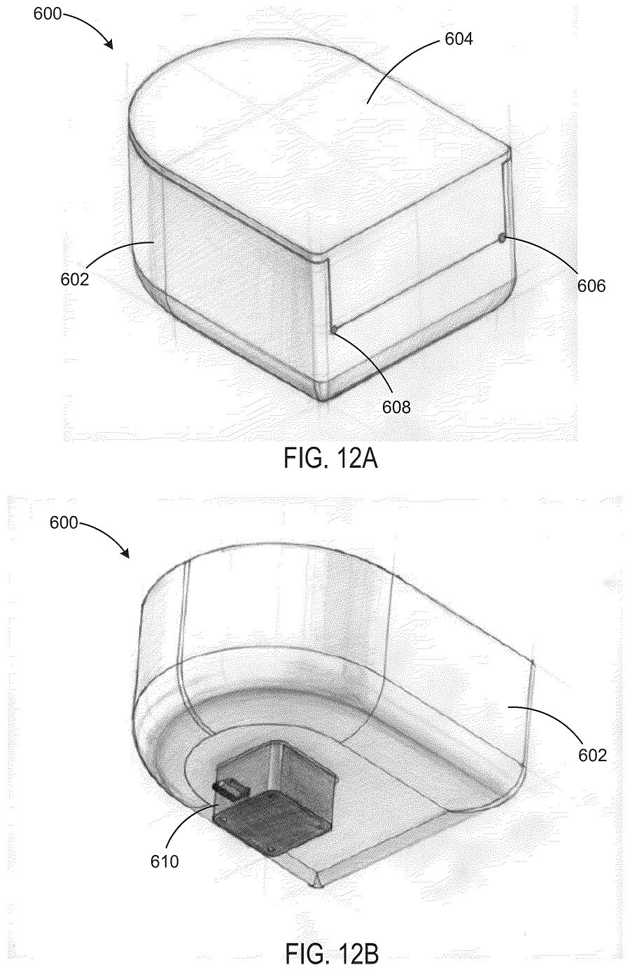

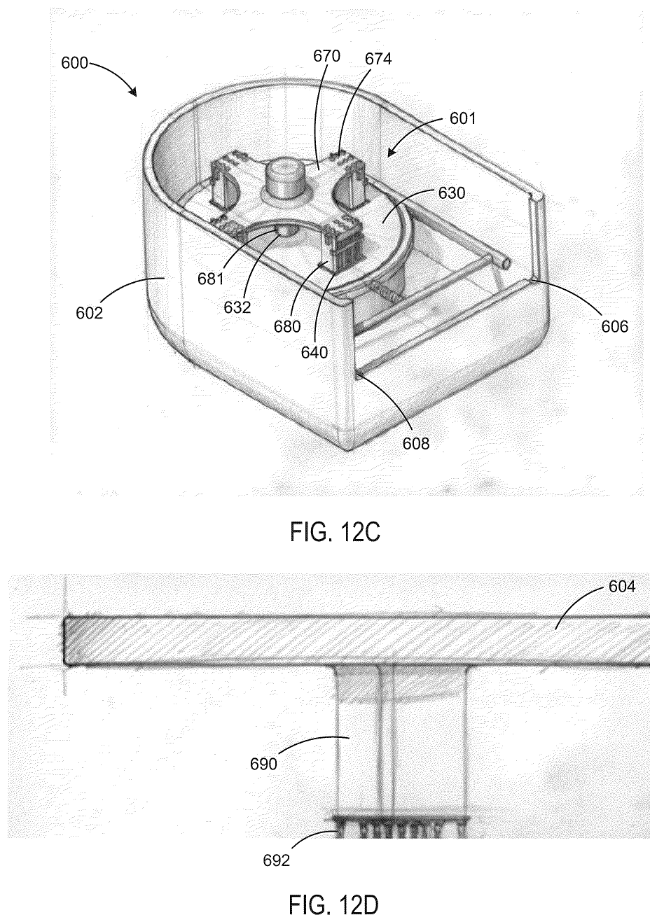

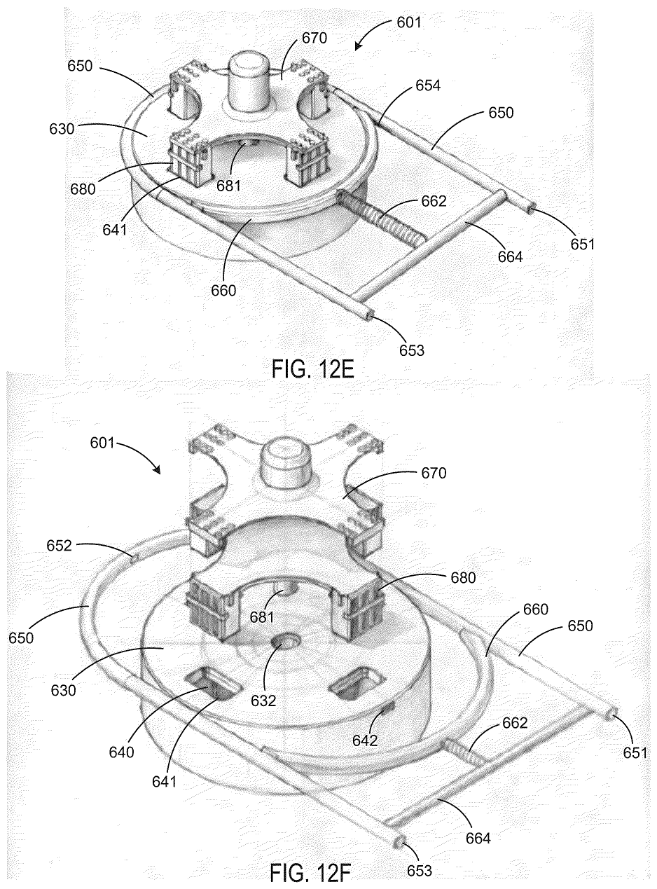

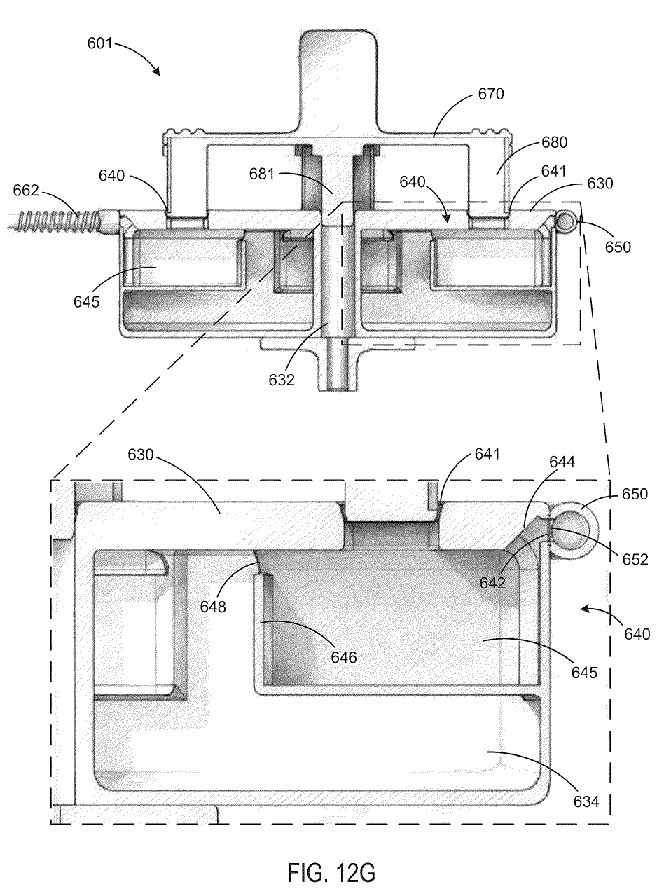

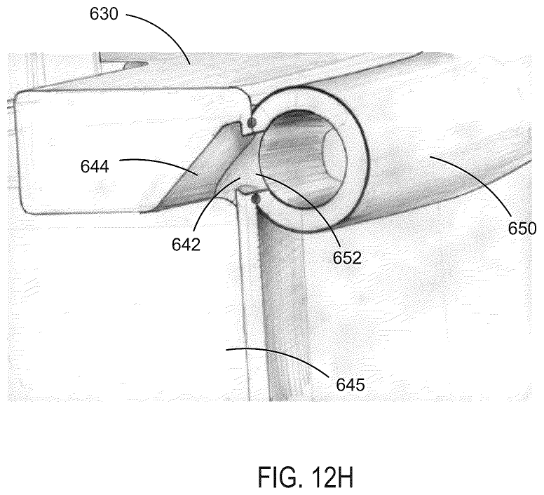

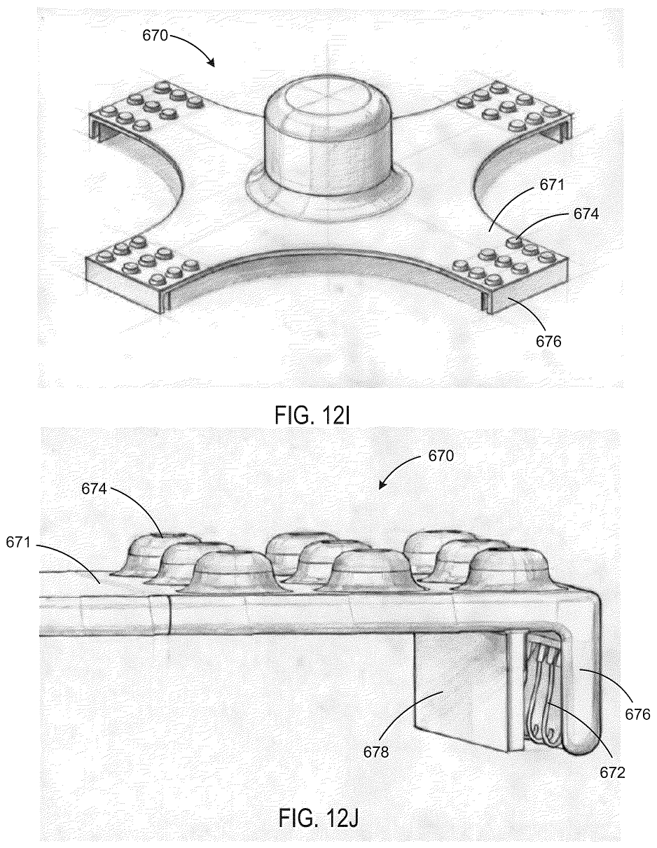

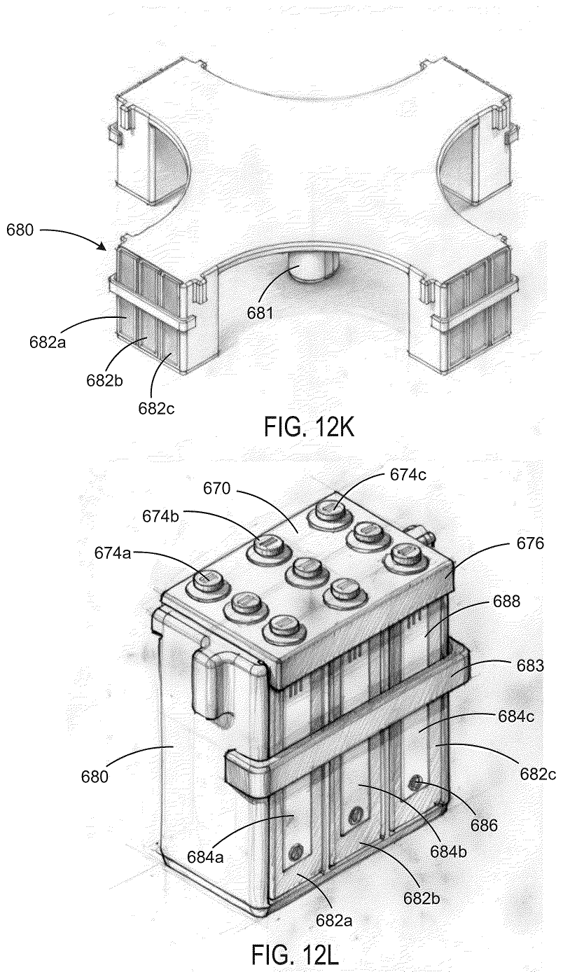

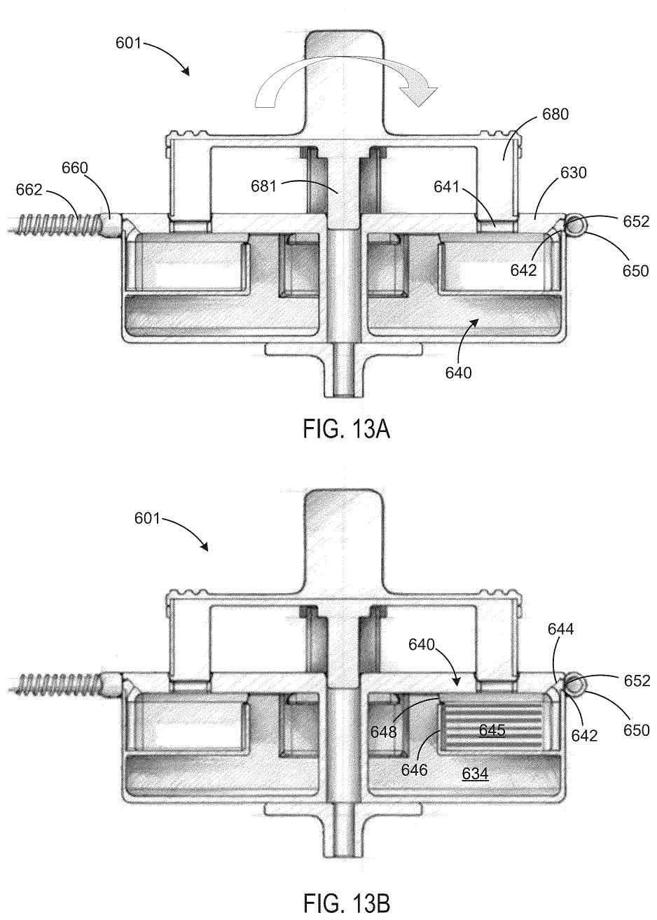

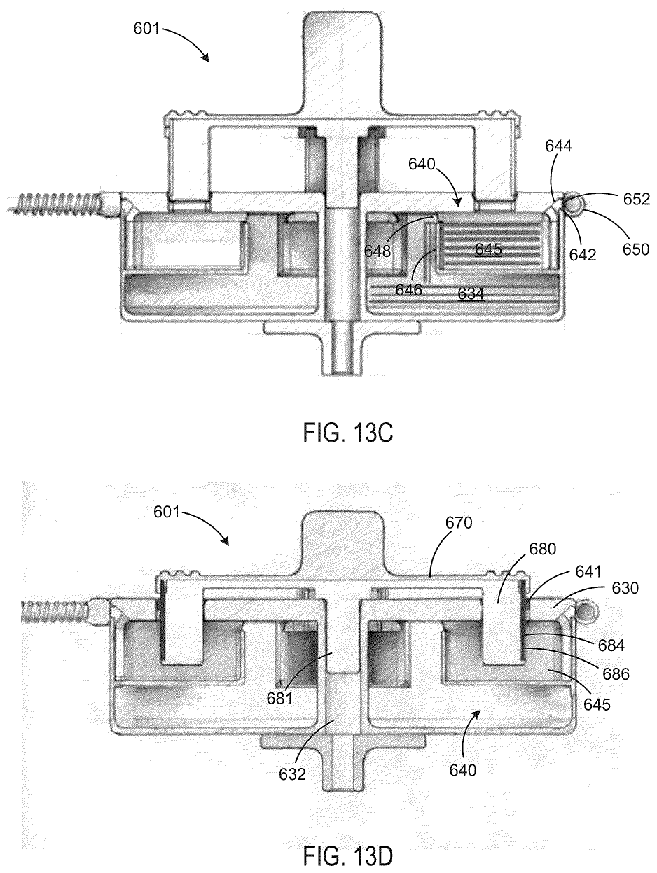

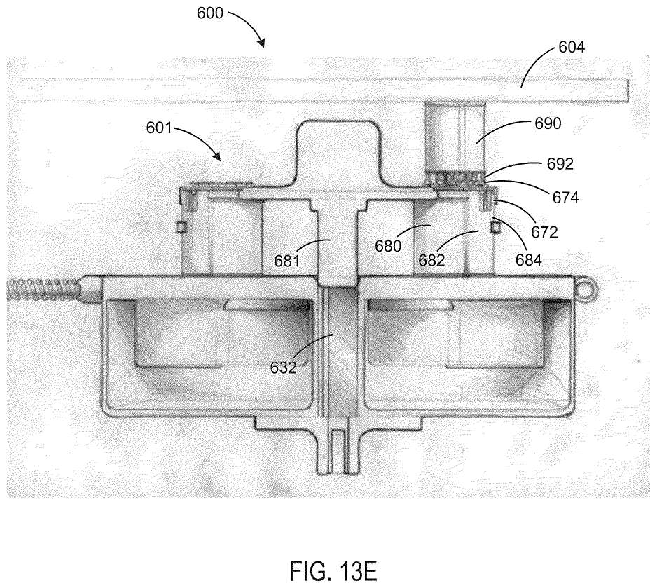

In addition, the case configured for coupling a cycler drain line to a drain bag line may include a sensor for measuring glucose concentration in the drained fluid. For example, the dialysate glucose concentration will gradually decrease due to absorption of glucose into the patient's bloodstream when inside the patient's peritoneal cavity, which may reduce fluid removal on dialysis. Accordingly, the controller may be programmed to cause drainage of fluid from the patient before the sensed dialysate glucose concentration within the patient drops below a predetermined threshold, thereby optimizing fluid removal. As another example, the case configured for coupling a cycler drain line to a drain bag line further may include a sensor for measuring creatinine levels, to thereby provide real time information on the clearance of creatinine during peritoneal dialysis. For example, creatinine leaves the patient's bloodstream to enter the dialysate during peritoneal dialysis, such that the longer the fluid remains in the peritoneal cavity, the more clearance of creatinine from the patient's bloodstream may be achieved. However, the longer dwell time may reduce fluid removal. Accordingly, the controller may be programmed to cause drainage of fluid from the patient when the sensed creatinine levels in the fluid within the patient reaches a predetermined threshold, thereby optimizing drainage based on the balance of fluid removal and efficiency of creatinine clearance. In some embodiments, automated sampling and monitoring of drained fluid for measuring, e.g., glucose, blood urea nitrogen (BUN), and/or creatinine concentration levels in the drained fluid may be provided via a designated case, as described in further detail below with regard to FIGS. 12A to 13E.

An exemplary workflow of the automated dialysis line connection system is provided. For example, the user first turns on the cycler, loads the cassette onto the cycler, and then turns on the automated dialysis line connection system. The user may then position the dialysis bags and drain bag in their allocated slots, and position the line connectors of the various dialysis lines in the appropriate cases. Once the proper dialysis lines and connectors have been properly positioned within the appropriate cases, as described above, the user may perform a single action to initiate the automated connection and optional automated clamping system. For example, upon commencement of the automated system, the automated connections of the cycler solution lines with the bag lines, the automated connection of the cycler drain line with the drain bag line, and the automated clamping of the unused cycler solution lines may occur simultaneously or sequentially. However, the automated connection of the cycler outlet line with the transfer set may not be commenced until confirmation that the priming phase, e.g., to remove any air bubbles within the dialysis lines, is complete. Accordingly, the automated dialysis line connection system may be configured to communicate with the cycler to thereby transmit a signal to the cycler to begin the priming phase, and to receive a signal indicative of the completion of the priming phase. Upon completion of the automated connections, the system further may transmit a signal to the cycler to initiate the dialysis treatment.

In some embodiments, the controller of the automated dialysis line connection system may be programmed to initiate the automated connection of the cycler patient line with the transfer set after a predetermined time period selected to ensure that the priming phase has been completed. Additionally, or alternatively, the information generated by the sensor for measuring the presence/level of liquid within the cycler patient line may be indicative of when the priming phase is complete, such that the controller may be programmed to initiate the automated connection of the cycler patient line with the transfer set when the sensed liquid level reaches a predetermined threshold.

Referring now to FIGS. 12A to 12L, an exemplary case for real-time, automated effluent sampling of fluid exiting the patient's body (e.g., dialysate effluent) via the drain line of the dialysis circuit during an automated peritoneal dialysis (APD) therapy for measuring one or more solute concentration levels thereof is provided. For example, the automated effluent sampling system may be fluidically coupled to the drain line in a manner preserving continuous gravity-driven flow to the drain bag or drain line extension tubing while enabling precise, controlled diversion of small effluent volumes for analysis. Case 600 may permit up to four discrete, single-use measurements throughout a typical peritoneal dialysis treatment session (e.g., one per sampling well and corresponding test strip holder), thereby enabling dynamic profiling of effluent solute concentrations. Moreover, case 600 may be used as a standalone device that may be used with any dialysis system for sampling and measuring dialysate effluent solute concentrations therein and/or may be operatively integrated with automated dialysis line connection system 100, as described in further detail below. For example, in some embodiments, case 600 may be used instead of case 500, as shown in FIG. 1, such that case 600 may be fluidically coupled to cycler drain line ML5 at one end, and to the drain bag (e.g., via drain bag line DL) or a drain line extension tubing, as desired for the ADP treatment.

As shown in FIG. 12A, case 600 may include housing 602, and lid 604 configured to enclose housing 602. Housing 602 may define an interior cavity sized and shaped to house automated effluent sampling system 601 therein, as shown in FIG. 12C. In addition, as shown in FIGS. 12A and 12C, case 600 may include first window 606 and second window 608, e.g., along a rear of housing 602, each sized and shaped to receive the tubing of a respective dialysis line therein in a manner that does not disrupt fluid flow therethrough, such that upon closure of lid 604, the respective dialysis lines will be maintained within first window 606 and second window 608. For example, first window 606 may be sized and shaped to receive cycler drain line ML5 therethrough and second window 608 may be sized and shaped to receive drain bag line DL therethrough. Alternatively, in some embodiments, first window 606 may be sized and shaped to receive a first portion of drain bag line DL therethrough (e.g., the portion of drain bag line DL fluidically coupled to cycler drain line ML5) and second window 608 may be sized and shaped to receive a second portion of drain bag line DL therethrough (e.g., the portion of drain bag line DL fluidically coupled to drain bag 112) or, alternatively, to a drain line extension tubing (e.g., for drainage to a location other than drain bag 112). Closing lid 604 will block pathogens from contacting the dialysis lines and connectors, thereby maintaining sterility.

Moreover, case 600 may include an integrated linear-rotary motor, e.g., linear-rotary motor 610, disposed on the underside of housing 602, as shown in FIG. 12B. Linear-rotary motor 610 may be operatively coupled to the controller of automated dialysis line connection system 100 for transmitting and receiving electrical signals therebetween, and may be configured to independently provide motion in two axes, e.g., rotary motion and linear motion. For example, as shown in FIG. 12C, the shaft of linear-rotary motor 610 may extend vertically upward through a central channel of a fluid collection chamber (e.g., channel 632 of fluid collection chamber 630) disposed within housing 602, and may be actuated to provide rotary motion of fluid collection chamber 630 relative to housing 602, and further may be independently actuated to provide linear motion of a test strip holder (e.g., test strip holders 680) in the vertical direction, e.g., via central column 681, for sampling dialysate effluent, as described in further detail below.

Accordingly, linear-rotary motor 610 may be actuated to linearly move test strip holders 680 vertically between a sampling configuration where test strip holders 680 are inserted into respective sampling wells (e.g., sampling wells 640) of fluid collection chamber 630, such that one or more solute concentration test strips disposed within test strip holders 680 may be submerged in dialysate effluent collected within the fluid collection chamber, and a monitoring configuration where test strip holders 680 are in electrical communication with one or more sensors for measuring the solute concentration levels of dialysate effluent via the test strips, as described in further detail below. For example, test strip holders 680 may be removably coupled to an interposer (e.g., L-shaped interposer 670) having a target pad (e.g., target pad 674) disposed on the top surface thereof and in electrical communication with the test strips within test strip holder 680 such that, in the monitoring configuration, target pad 674 is in electrical contact with one or more sensors disposed on the underside of lid 604 to thereby electrically connect the test strips to the sensors. In some embodiments, interposer 670 and test strip holders 680 may be linearly moved to an intermediate vertical position (e.g., default position) where test strip holders 680 are not disposed/submerged within the respective sampling wells, and where target pad 674 is not in electrical contact with the one or more sensors.

Accordingly, as shown in FIG. 12D, case 600 may include one or more sensors (e.g., sensor 690) coupled to the underside of lid 604, e.g., in alignment with one or more corresponding test strip holders 680, and configured to obtain electrical signals generated by the test strips, e.g., via target pad 674, as described in further detail below. For example, sensor 690 may comprise a horizontal array of pogo-pins (e.g., pogo-pin array 692) extending downward therefrom to facilitate and maintain electrical contact with target pad 674 of interposer 670 and, accordingly, electrically connect sensor 690 to the test strips within test strip holder 680 in the monitoring configuration. Pogo-pin array 692 may be spring loaded to thereby maintain constant electrical contact with target pad 674, e.g., in the event of uneven pressure contact. Preferably, pogo-pin array 692 are arranged in a parallel array with wider spacing to ensure reliable contact and provide adequate spring force.

Referring now to FIGS. 12E to 12L, an exemplary automated effluent sampling system of case 600 is provided. FIG. 12E shows the components of automated effluent sampling system 601 assembled together in the monitoring configuration (with sensor 690 not shown for brevity), and FIG. 12F shows an exploded view of the components of automated effluent sampling system 601. As shown in FIGS. 12E and 12F, automated effluent sampling system 601 may include fluid collection chamber 630, interposer 670, and test strip holder 680, which may be coaxially locked together, such that they are rotated together as a unit, e.g., upon actuation of motor 610. Moreover, interposer 670 and test strip holder 680 may be removably coupled together, such that they are linearly moved together as a unit (e.g., between the sampling and monitoring configurations) relative to and independent of fluid collection chamber 630, e.g., via central column 681 upon actuation of motor 610.

As shown in FIGS. 12E and 12F, automated effluent sampling system 601 may include effluent tube 650 configured to wrap at least partially around the outer circumference of fluid collection chamber 630 (e.g., along 180 degrees), and having inlet 651 configured to be fluidically coupled to a drain line through which dialysis effluent flows (e.g., cycler drain line ML5) and outlet 653 configured to be fluidically coupled to the drain bag (e.g., drain bag 112 via drain bag line DL) or, alternatively, to a drain line extension tubing for drainage elsewhere other than a drain bag. For example, effluent tube 650 may comprise a first portion that extends longitudinally from inlet 651 towards fluid collection chamber 630, a second curved portion having a curved profile corresponding to the curvature of the sidewall of fluid collection chamber 630, and a third portion that extends longitudinally from fluid collection chamber 630 towards outlet 653.

As shown in FIG. 12F, at least the curved portion of effluent tube 650 may comprise sampling port 652 sized and shaped to permit effluent fluid to flow therethrough when unobstructed. For example, effluent fluid may flow from within the lumen of effluent tube 650 and into a sampling well of fluid collection chamber 630 (e.g., sampling well 640) via a corresponding fluid inlet (e.g., lateral inlet 642) of the sampling well when sampling port 652 is sealingly and fluidically coupled to the lateral inlet, as described in further detail below. Preferably, sampling port 652 may have a rectangular profile, whereby its long axis is oriented tangentially along the length of effluent tube 650 (e.g., parallel with the longitudinal axis of effluent tube 650) to thereby facilitate fluid flow from effluent tube 650 to fluid collection chamber 630. Moreover, fluid flow may not be permitted through sampling port 652 when sampling port 652 is obstructed, e.g., sealed against the sidewall of fluid collection chamber 630 when sampling port 652 is not circumferentially aligned with lateral inlet 642.

Moreover, automated effluent sampling system 601 may comprise sliding lock 660 configured to transition between an unlocked state where sliding lock 660 is disengaged from fluid collection chamber 630, and a locked state where sliding lock 660 applies a force against fluid collection chamber 630 to thereby prevent rotation of fluid collection chamber 630 and facilitate fluidic sealing of the curved portion of effluent tube 650 against fluid collection chamber 630, e.g., when sampling port 652 and lateral inlet 642 are aligned to thereby permit flow of effluent fluid from effluent tube 650 into the corresponding sampling well of fluid collection chamber 630. For example, sliding lock 660 may have a curved profile (e.g., a semicircular shape) that corresponds to the curvature of the sidewall of fluid collection chamber 630.

As shown in FIG. 12E, the longitudinally extending portions of effluent tube 650 may each comprise a longitudinally extending track (e.g., track 654) configured to guide movement of sliding lock 660 between the locked and unlocked states. Sliding lock 660 may be actuated to transition between the locked and unlocked states via a motor (not shown). In addition, spring 662 attached perpendicularly to cross bar 664 extending horizontally between and the longitudinally extending portions of effluent tube 650, as shown in FIG. 12F, may apply a consistent axial force to sliding lock 660 to facilitate fluidic sealing of the curved portion of effluent tube 650 against fluid collection chamber 630, as described above.

FIG. 12G is a cross-sectional view of automated effluent sampling system 601 when effluent tube 650 is sealed against fluid collection chamber 630 (e.g., via sliding lock 660) and interposer 670 and test trip holder 680 are in the monitoring state. Fluid collection chamber 630 may comprise a number of sampling wells (e.g., sampling wells 640) that corresponds with the number of test trip holders 680. For example, fluid collection chamber 630 may have four sampling wells 640 equally spaced 90 degrees apart and aligned with the four test trip holders 680, as shown in FIGS. 12E and 12F. As will be understood by a person having ordinary skill in the art, automated effluent sampling system 601 may have less or more than four test strip holders and sampling wells, e.g., one, two, three, five, or more test strip holders and sampling wells.

Each sampling well 640 may define an internal cavity sized and shaped to collect a precise optimal predetermined volume of effluent fluid within collection zone 645, while diverting excess effluent fluid (e.g., overflow) to waste reservoir 634. For example, as shown in FIG. 12G, each sampling well 640 may comprise lateral inlet 642 in fluidic communication with collection zone 645 and configured to be aligned with sampling port 652 of effluent tube 650, e.g., upon rotation of fluid collection chamber 630 via motor 610, to thereby fluidically couple effluent tube 650 and collection zone 645 of sampling well 640 and permit effluent fluid to flow from effluent tube 650 into sampling well 640, e.g., via hydrostatic pressure. Sampling well 640 may comprise deflector plate 644 having an angled profile configured to direct effluent fluid from effluent tube 650 into collection zone 645 when lateral inlet 642 is aligned with and sealingly and fluidically coupled to sampling port 652, as shown in FIG. 12H. For example, deflector plate 644 may be configured to span the full width of the respective sampling well.

Moreover, as shown in FIG. 12G, each sampling well 640 may comprise overflow weir 646 that extends vertically upward from a bottom of collection zone 645 to thereby at least partially define a sidewall of collection zone 645, and which further defines overflow channel 648 at the upper portion of collection zone 645. Overflow channel 648 may be in fluidic communication with waste reservoir 634 to thereby permit excess effluent fluid to overflow from collection zone 645 to waste reservoir 634, as necessary. In some embodiments, waste reservoir 634 may be disposed at least partially beneath collection zone 645, as shown in FIG. 12G. In addition, waste reservoir 634 may be emptied manually by the user, as necessary. Accordingly, the geometry (e.g., height) of overflow weir 646 may be selected to define the predetermined maximum volume of collection zone 645. In addition, each sampling well 640 may comprise inlet 641 sized and shaped to receive a corresponding test strip holder 680 therethrough, e.g., upon linear motion of transposer 670 and test strip holder 680 via motor 610 and central column 681, as described above. Accordingly, in the sampling configuration, test strip holders 680 may be disposed within collection zone 645 of sampling wells 640 to thereby submerge at least a portion of the test strips of test strip holders 680 within the effluent fluid collected within collection zone 645.

As described above, the shaft of linear-rotary motor 610 may be mechanically coupled to fluid collection chamber 630 and to central column 681 of test strip holder 680, which extends at least partially through central channel 632 of fluid collection chamber 630 in a coaxially locked manner, as shown in FIG. 12G, and which in turn may be mechanically coupled to interposer 670, such that actuation of motor 610 may cause synchronous rotation of fluid collection chamber 630, test strip holder 680, and interposer 670. Accordingly, each test strip holder 680 may be permanently aligned with a corresponding inlet 641 of a respective sampling well 640. Further, as the shaft of linear-rotary motor 610 may be mechanically coupled to central column 681 of test strip holders 680, which in turn may be mechanically coupled to interposer 670, actuation of motor 610 may cause synchronous linear movement of test strip holders 680 and interposer 670 relative to and independent of fluid collection chamber 630 (e.g., when interposer 670 is removably coupled to test strip holders 680). In addition, interposer 670 may be moved linearly relative to test strip holders 680 when interposer 670 is not coupled to test strip holders 680, e.g., for cleaning/maintenance of interposer 670 and test strip holders 680.

As discussed above, interposer 670 and test strip holder 680 may be coupled together, such that they are moved linearly together independent of fluid collection chamber 630, e.g., upon actuation of motor 610 via central column 681. As shown in FIG. 12I, interposer 670 may comprise a number of branches corresponding to the number of branches of test strip holder 680, as shown in FIG. 12K, e.g., four branches 671 extending radially outward and equally spaced apart (e.g., by 90 degrees). Each branch 671 of interposer 670 may comprise an L-shaped profile, and may be configured to be aligned with and mechanically coupled to the corresponding branches of test strip holder 680 (e.g., via a snap fit connection).

As shown in FIG. 12J, the end regions of each branch 671 of interposer 670 may comprise an L-shaped profile defined by lip portion 676 extending perpendicularly downward from branch 671. Each branch 671 further may include inner protrusion 678 extending perpendicularly downward from branch 671, parallel with lip portion 676, to thereby define a gap therebetween. The gap may be sized and shaped to receive at least an upper portion of a corresponding test strip holder 680 to thereby facilitate mechanical coupling of test strip holder 680 to interposer 670, e.g., via a snap fit connection. As shown in FIG. 12J, each branch 671 of interposer 670 may comprise a plurality of spring fingers 672 (e.g., disposed on an inner side of lip portion 676) configured to press tightly against the test strips disposed within test strip holder 680 (e.g., against contact pad 688 of test strips 684, as described in further detail below with regard to FIG. 12L). Moreover, spring fingers 672 may be electrically connected to target pad 674 disposed on the upper surface of interposer 670, to thereby electrically connect the test strips to pogo-pin array 692 of sensor 690 in the monitoring configuration, as described above.

As shown in FIG. 12K, automated effluent sampling system 601 may include four test strip holders 680, each configured to house a set of test strips (e.g., three test strips) within a dedicated test strip compartment (e.g., test strip compartments 682a, 682b, 682c, collectively referred to herein as test strip compartments 682), which may be separated from one another via insulating spacers. As shown in FIG. 12L, each test strip compartment 682a, 682b, 682c may be configured to receive a dedicated test strip (e.g., glucose test strip 684a, creatinine test strip 684b, or blood urea nitrogen (BUN) test strip 684c) therein in parallel, such that reactive zone 686 of each test strip is positioned towards a bottom portion of the respective test strip compartment, and contact pad 688 of each test strip is positioned towards an upper portion of the respective test strip compartment in electrical contact with corresponding spring fingers 672, as described above. For example, the user may insert the end of the respective test strip having contact pad 688 upward through retainer 683 until it is sandwiched by spring fingers 672, such that the end of the respective test strip with reactive zone 686 is disposed at the lower portion of test strip holder 680 (e.g., below retainer 683), as shown in FIG. 12L. Moreover, as shown in FIG. 12L when test strip holder 680 is coupled to interposer 670, a corresponding set of contacts of target pad 674 (e.g., contacts 674a, 674b, 674c) may be disposed over and aligned with a corresponding test strip compartment 682a, 682b, 682c, respectively, while spring fingers 672 are pressed tightly against the respective contact pads 688 of test strips 684a, 684b, 684c to thereby ensure constant contact therebetween.

Referring now to FIGS. 13A to 13E, an exemplary method for automated effluent sampling of dialysate effluent using automated effluent sampling system 601 of case 600 is provided. First, the user may insert a set of test strips (e.g., glucose test strip 684a, creatinine test strip 684b, BUN test strip 684c) into the corresponding test strip compartments (e.g., test strip compartments 682a, 682b, 682c) of one or more of the four test strip holders 680. For example, when all four test strip holders 680 receive a complete set of test strips, 12 test strips will be used, e.g., four of each of glucose test strip 684a, creatinine test strip 684b, and BUN test strip 684c. Moreover, the user may connect a first end of effluent tube 650 (e.g., inlet 651) to a drain line (e.g., cycler drain line ML5) and the other end of effluent tube 650 (e.g., outlet 653) to the drain bag (e.g., drain bag 112 via drain bag line DL) or, alternatively, to a drain line extension tubing.

As shown in FIG. 13A, linear-rotary motor 610 may be actuated to cause rotation of fluid collection chamber 630 and, accordingly test strip holder 680 and interposer 670 via central channel 632, until lateral inlet 642 of sampling well 640 is positioned in alignment with sampling port 652 of effluent tube 650. Preferably, at this stage, test strip holders 680 and interposer 670 are in a first raised position where test strip holders 680 are not disposed within sampling wells 640 of fluid collection chamber 630. In some embodiments, the first raised position (e.g., the default position) may be lower than the monitoring configuration, such that contact pad 674 of interposer 670 is not in electrical contact with pogo-pin array 692 of sensor 690. Alternatively, the first raised position may be the monitoring configuration. Sliding lock 660 may then be actuated to transition to the locked state, as shown in FIG. 13A, where sliding lock 660 presses tightly against fluid collection chamber 630, which in turn causes the curved portion of effluent tube 650 to press tightly against the other side of fluid collection chamber 630, to thereby create a fluid tight seal between lateral inlet 642 and sampling port 652. As described above, spring 662 of sliding lock 660 further may apply a spring force to facilitate sealing of effluent tube 650 against fluid collection chamber 630.

Accordingly, when sampling port 652 is unobstructed and in fluid communication with lateral inlet 642, hydrostatic pressure may cause effluent fluid to flow from effluent tube 650 into collection zone 645 of sampling well 640 via sampling port 652 and lateral inlet 642, as shown in FIG. 13B. As described above, deflector plate 644 may facilitate/guide effluent fluid flow from effluent tube 650 into collection zone 645, e.g., by preventing effluent fluid from splashing outside of sampling well 640. Linear-rotary motor 610 may be instructed (e.g., via the controller of system 100) to maintain fluid collection chamber 630 in this stationary position for at least a predetermined time period to thereby permit effluent fluid to fill collection zone 645, as shown in FIG. 13B. As described above, any excess effluent fluid may overflow into waste reservoir 634 via overflow channel 648 defined by overflow weir 646, as shown in FIG. 13C.

After the predetermined time period, sliding lock 660 may be transitioned to the unlocked state where sliding lock 660 disengages fluid collection chamber 630, thereby loosening the seal between sampling port 652 and lateral inlet 642, and permitting rotation of fluid collection chamber 630. Linear-rotary motor 610 may be actuated to rotate fluid collection chamber 630 to an angular position where sampling port 652 of effluent tube 650 is obstructed by the sidewall of fluid collection chamber 630 (e.g., sampling port 652 is not aligned with any of the lateral ports of fluid collection chamber 630), such that no effluent fluid may flow out of sampling port 652. Sliding lock 660 may be transitioned to the locked positioned to maintain sampling port 652 in the obstructed state against the sidewall of fluid collection chamber 630.

As shown in FIG. 13D, linear-rotary motor 610 may be actuated to cause test strip holders 680 and interposer 670 to move linearly downward to a second lowered position relative to fluid collection chamber 630 to thereby descend test strip holders 680 into corresponding sampling wells 640 of fluid collection chamber 630 via corresponding inlets 641, e.g., the sampling configuration. Accordingly, in the sampling configuration, reactive zones 686 of each test strip 684 within each test strip holder 680 will be immersed in the effluent fluid collected within collection zone 645 of sampling well 640. Linear-rotary motor 610 may be instructed (e.g., via the controller of system 100) to maintain test strip holders 680 in the sampling configuration for at least a predetermined dwell time period to thereby permit test strips 684 to complete a chemical reaction with the effluent fluid.

After the predetermined dwell time period, linear-rotary motor 610 may then be actuated in the opposite direction to cause test strip holders 680 and interposer 670 to move linearly upward to a third raised position relative to fluid collection chamber 630 to thereby ascend test strip holders 680 out of sampling wells 640, e.g., the monitoring configuration, such that test strip holders 680 and, accordingly test strips 684 are no longer submerged in effluent fluid within collection zone 645, and contact pad 674 of interposer 670 are in electrical contact with pogo-pin array 692 of sensor 690, as shown in FIG. 13E. As described above, pogo-pin array 692 may be spring-loaded to thereby maintain a strong electrical connection between sensor 690 and contact pad 674 of interposer 670, e.g., even if contact pad 674 of interposer 670 is not evenly pressed against pogo-pin array 692.

Accordingly, sensor 690 may generate and transmit data indicative of the electrical signals received from test strip holders 680 (e.g., via spring fingers 672, contact pad 674, and pogo-pin array 692), which are indicative of the corresponding solute concentration levels (e.g., glucose, BUN, creatinine) as measured by the corresponding test strips 684, to an external computing device (e.g., the controller of system 100 or another external computing device) programmed to record, process, and analyze the data to thereby measure the corresponding solute concentration levels of the effluent fluid. As described above, data indicative of the measured solute concentration levels may then be processed by the controller of system 100 to determine whether the measure solute concentration levels are within respective predetermined acceptable ranges. For example, the measured glucose concentration is correlated with ultrafiltration and net fluid removal, while the measured BUN and creatinine concentrations are correlated with solute clearance. Accordingly, the controller of system 100 may use this information from a previous cycle to dynamically adjust the peritoneal dialysis treatment by causing the cycler to initiate drainage of fluid either earlier or later during a subsequent cycle.

Linear-rotary motor 610 may then be actuated to cause test strip holders 680 and interposer 670 to move linearly downward to the second lowered position relative to fluid collection chamber 630, such that contact pad 674 of interposer 670 is no longer in electrical contact with pogo-pin array 692 of sensor 690, and rotation of fluid collection chamber 630 is permitted. Accordingly, the steps above may be repeated to fill the collection zone of a subsequent sampling well 640 of fluid collection chamber 630 with effluent fluid from effluent tube 650 for sampling and monitoring. For example, linear-rotary motor 610 may then be actuated to cause further rotation of fluid collection chamber 630 to thereby align lateral inlet 642 of another sampling well of fluid collection chamber 630 with sampling port 652 of effluent tube 650, and permit effluent fluid from effluent tube 650 to fill the collection zone of that sampling well. This process may be repeated until all of the sampling wells have been utilized.

Referring now to FIGS. 14A to 14I, an exemplary compact dispensing system for automated storage and dispensing of dialysate bags used in an automated peritoneal dialysis (APD) treatment is provided. Each APD treatment session typically requires multiple dialysate bags, often with different concentrations tailored to the patient's prescription. The compact automated storage and dispensing system may include a central main assembly and a plurality of modular magazines (e.g., three modular magazines) that may each be detachable and transportable, allowing for convenient loading and storage of dialysate bags therein. Thus, the compact automated storage and dispensing system may reduce patients'physical strain associated with each APD treatment, while minimizing space requirement for dialysate bags storage. Accordingly, the compact system may particularly be suitable for space-constrained environments such as small bedrooms or apartments and for dialysis patients with limited physical strength or mobility. The system may be used as a standalone device that may be used with any dialysis treatment system and/or may be integrated with any of the connection cases and dialysate samplers of automated dialysis line connection system 100, as described above.

As shown in FIGS. 14A and 14B, compact automated storage and dispensing system 700 may include a plurality of modular magazine carts (e.g., mobile columns 710a, 710b, 710c, collectively referred to herein as mobile columns 710), each sized and shaped to receive a stack of a predetermined number of dialysate bags therein for storage and automated dispensing for use during an APD treatment, and transfer module 730 configured to automatically receive and dispense dialysis bags received from mobile columns 710 for use during the APD treatment. For example, transfer module 730 may be supported via a plurality of vertical stands 702 defining a plurality of docking stations 704 therebetween (e.g., three docking stations), each docking station 704 sized and shaped to receive a mobile column therein, such that mobile columns 710 may be disposed underneath transfer module 730 for coordination of retrieval and dispensing of bags during operation, as described in further detail below.

Compact automated storage and dispensing system 700 may be integrated with automated dialysis line connection system 100, as shown in FIG. 14A, where the connection cases (e.g., cases 200, 300, 500) and optionally, the dialysate sampler (not shown) may be coupled to transfer module 730 to provide easy access thereto for automated coupling with the dialysate bags dispensed via transfer module 730 for use during the APD treatment. In some embodiments, compact automated storage and dispensing system 700 may be a standalone device, as shown in FIG. 14B, that may be used with any dialysis treatment system. As shown in FIGS. 14A and 14B, compact automated storage and dispensing system 700 may include a graphical user interface (e.g., GUI 701) configured to be operatively coupled to the controllers of each of mobile columns 710, for electrical communication therebetween, as described in further detail below. GUI 701 may be disposed on or, otherwise, integrated with transfer module 730.

As shown in FIG. 14C, each of mobile columns 710a, 710b, 710c may be removable from the respective docking station (e.g., docking stations 704a, 704b, 704c, collectively referred to herein as docking stations 704) of system 700 and transportable for convenient loading and storage of dialysate bags therein. For example, as shown in FIGS. 14D and 14E, each mobile column 710 may comprise a plurality of swivel wheels 714 configured to provide mobility to mobile column 710, and further may comprise handle 718 configured to facilitate movement of mobile column 710 by the user. In some embodiments, handle 718 may be retractable between a collapsed state (e.g., for storage), and an extended state, as shown in FIG. 14E, for efficient gripping by the user during mobility. In addition, each mobile column 710 may comprise body 712 defining an internal compartment sized and shaped to receive a stack of a predetermined number of dialysate bags (e.g., five dialysate bags), e.g., in a predefined aligned manner.

As shown in FIG. 14E, mobile column 710 may comprise controller 711 (e.g., a touchscreen display) configured to receive and display user input indicative of, for example, the dialysate concentrations of the dialysate bags (e.g., dialysate bag 110) loaded therein by the user, which may be different from the dialysate bags stored in the other mobile columns, as well as the quantity of dialysate bags stored therein. Moreover, mobile column 710 may comprise connector port 716, as shown in FIG. 14D, for electrical connection with a corresponding connector port of GUI 701 of transfer module 730, to thereby permit data transfer therebetween. Accordingly, GUI 701 further may display information indicative of the dialysate concentrations of the dialysate bags stored within each of mobile columns 710, as well as the quantity of dialysate bags.

FIG. 14F shows the interior of body 712 of mobile column 710 and, particularly, the loading and dispensing mechanism disposed therein. As shown in FIG. 14F, mobile column 710 may comprise electronics box 721 having control circuitry and memory, e.g., for electrical communication between controller 711 and GUI 701, as well as an internal battery configured to power the electronic components of mobile column 710, such as motor 722, as described in further detail below. For example, the internal battery of electronics box 721 may be charged with power received via connector port 716. Moreover, mobile column 710 may comprise motor 722 operatively coupled to ball screw shaft 724 extending vertically upward therefrom, the ball screw shaft 724 slidably coupled to shelf 720 via nut 726 (e.g., a threaded nut) mounted to shelf 720. Shelf 720 may be sized and shaped to support a plurality of dialysate bags thereon within body 712 in a manner such that motion of the dialysate bags (other than the ascending and descending motion provided via shelf 720) is minimized.

Accordingly, motor 722 may be actuated to cause rotation of ball screw shaft 724 in a first rotational direction, thereby causing linear movement of nut 726 and, accordingly, shelf 720 along ball screw shaft 724 in a first vertical direction (e.g., upward), and further may be actuated to cause rotation of ball screw shaft 724 in a second rotational direction opposite to the first rotational direction, thereby causing linear movement of nut 726 and, accordingly, shelf 720 along ball screw shaft 724 in a second vertical direction opposite to the first vertical direction (e.g., downward). In addition, as shown in FIG. 14F, mobile column 710 may comprise sensor 728 (e.g., an infrared sensor) disposed at an upper portion of mobile column 710 and configured to detect if the top-most slot for the dialysate bag within body 712 is occupied or not. For example, if no dialysate bag is detected in the top-most slot within body 712 via sensor 728, motor 722 may be actuated to ascend shelf 720 to a vertical position where a dialysate bag is detected within the top-most slot. Further, if shelf 720 is ascended to the top-most vertical position where a dialysate bag would otherwise be detected, if present, and no dialysate bag is detected, the system may determine that the mobile column is empty of dialysate bags. Sensor 728 may be operatively coupled to electronics box 721 for transmitting data to controller 711 and/or GUI 701 for coordinating dispensing of the dialysate bags stored therein. Accordingly, controller 711 and/or GUI 701 may be programmed to provide an alert (e.g., an audible and/or visual alert) to thereby notify the user when a particular mobile column is empty and needs to be refilled.

Referring now to FIGS. 14G to 14I, transfer module 730 is provided in further detail. As shown in FIG. 14G, transfer module 730 may comprise a top surface (e.g., left top surface 732a and right top surface 732b) sized and shaped to receive dialysate bags dispensed from mobile columns 710 thereon. The top surface of transfer module 730 may comprise access window 734, which may be centrally disposed between left top surface 732a and right top surface 732b, as shown in FIG. 14G, and which may be sized and shaped to permit a dialysate bag to be advanced therethrough. Moreover, transfer module 730 may include retractable cover 736 configured to be actuated to transition between an open state where passage through access window 734 is permitted, and a closed state where access window 734 is obstructed by cover 736. For example, retractable cover 736 may configured to slide horizontally relative to access window 734, e.g., via a dedicated motor and ball screw assembly (not shown). Additionally, transfer module 730 further may include one or more sensors (e.g., left load cell 738a disposed on left top surface 732a and right load cell 738b disposed on right top surface 732b) configured to detect a weight thereon to determine whether a dialysate bag is currently disposed on left top surface 732a and/or right top surface 732b. As shown in FIG. 14G, GUI 701, e.g., a OLED touchscreen device, may be disposed on the top surface of transfer module 730 to provide easy access by a user, e.g., for receiving user input commands to control operation of system 700, mobile columns 710, and/or transfer module 730, as well as easy observation of data displayed thereon.

FIGS. 14H and 14I show the interior of transfer module 730 and, particularly, the dispensing mechanism disposed therein. As shown in FIGS. 14H and 14I, transfer module 730 may comprise shelf 740 sized and shaped to support a dialysate bag thereon, left push arm 742 configured to push a dialysate bag in the rightward horizontal direction, and right push arm 744 configured to push a dialysate bag in the leftward horizontal direction. Shelf 740 may be configured to be moved in two axes, e.g., linear horizontal motion and linear vertical motion. For example, shelf 740 may be mounted to nut 750 (e.g., a threaded nut), which may be slidably coupled to vertical ball screw shaft 748 operatively coupled to and extending vertically from motor 746. Accordingly, motor 746 may be actuated (e.g., via the controller of GUI 701) to cause rotation of ball screw shaft 748 in a first rotational direction, thereby causing linear movement of nut 750 and, accordingly, shelf 740 along ball screw shaft 748 in a first vertical direction (e.g., upward), and further may be actuated to cause rotation of ball screw shaft 748 in a second rotational direction opposite to the first rotational direction, thereby causing linear movement of nut 750 and, accordingly, shelf 740 along ball screw shaft 748 in a second vertical direction opposite to the first vertical direction (e.g., downward). For example, shelf 740 may be moved along the vertical direction between a first loading position where shelf 740 is vertically aligned with the bottom surface of a dialysate bag dispensed from a respective mobile column 710 (e.g., above the upper end of the respective mobile column 710), and a second dispensing position where shelf 740 is vertically aligned/flushed with the top surface of transfer module 730, such that the dialysate bag disposed thereon is advanced through access window 734 (e.g., when cover 736 is in the open state) and in line with left top surface 732a and right top surface 732b.

For linear horizontal motion, the shelf assembly comprising shelf 740, nut 750, vertical ball screw shaft 748, and motor 746 may be mounted to nut 756 (e.g., a threaded nut), which may be slidably coupled to horizontal ball screw shaft 754 operatively coupled to and extending horizontally from motor 752. Motor 752 and horizontal ball screw shaft 754 may be affixed to a wall of transfer module 730. Accordingly, motor 752 may be actuated (e.g., via the controller of GUI 701) to cause rotation of ball screw shaft 754 in a first rotational direction, thereby causing linear movement of nut 756 and, accordingly, shelf 740 along ball screw shaft 748 in a first horizontal direction (e.g., rightward), and further may be actuated to cause rotation of ball screw shaft 754 in a second rotational direction opposite to the first rotational direction, thereby causing linear movement of nut 756 and, accordingly, shelf 740 along ball screw shaft 748 in a second horizontal direction opposite to the first horizontal direction (e.g., leftward). Horizontal ball screw shaft 754 may span the entire horizontal width of transfer module 730 to thereby permit horizontal translation of shelf 740 between a leftmost horizontal position over mobile column 710a and a rightmost horizontal position over mobile column 710c.

Left push arm 742 may be configured to translate linearly along the horizontal direction relative to shelf 740. For example, left push arm 742 may be mounted to nut 762 (e.g., a threaded nut), which may be slidably coupled to horizontal ball screw shaft 760 operatively coupled to and extending horizontally from motor 758. Motor 758 and horizontal ball screw shaft 760 may be affixed to a wall of transfer module 730. Accordingly, motor 758 may be actuated (e.g., via the controller of GUI 701) to cause rotation of ball screw shaft 760 in a first rotational direction, thereby causing linear movement of nut 762 and, accordingly, left push arm 742 along ball screw shaft 760 in a first horizontal direction (e.g., rightward), and further may be actuated to cause rotation of ball screw shaft 760 in a second rotational direction opposite to the first rotational direction, thereby causing linear movement of nut 762 and, accordingly, left push arm 742 along ball screw shaft 760 in a second horizontal direction opposite to the first horizontal direction (e.g., leftward). Horizontal ball screw shaft 760 may have a length spanning across at least the width of mobile column 710a, such that left push arm 742 may be horizontally translated at least between a leftmost horizontal position to the left of the mobile column 710a and a central position to the left of mobile column 710b (e.g., left of access window 734).

Right push arm 744 similarly may be configured to translate linearly along the horizontal direction relative to shelf 740. For example, right push arm 744 may be mounted to nut 766 (e.g., a threaded nut), which may be slidably coupled to horizontal ball screw shaft 764 operatively coupled to and extending horizontally from motor 763. Motor 763 and horizontal ball screw shaft 764 may be affixed to a wall of transfer module 730. Accordingly, motor 763 may be actuated (e.g., via the controller of GUI 701) to cause rotation of ball screw shaft 764 in a first rotational direction, thereby causing linear movement of nut 766 and, accordingly, right push arm 744 along ball screw shaft 764 in a first horizontal direction (e.g., leftward), and further may be actuated to cause rotation of ball screw shaft 764 in a second rotational direction opposite to the first rotational direction, thereby causing linear movement of nut 766 and, accordingly, right push arm 744 along ball screw shaft 764 in a second horizontal direction opposite to the first horizontal direction (e.g., rightward). Horizontal ball screw shaft 764 may have a length spanning across at least the width of mobile columns 710b and 710c, such that right push arm 744 may be horizontally translated at least between a rightmost horizontal position to the right of the mobile column 710c and a central position to the left of mobile column 710b (e.g., left of access window 734). As will be understood by a person having ordinary skill in the art, the sliding mechanisms of left push arm 742 and right push arm 744 may be switched, such that right push arm 744 may be horizontally translated between the rightmost horizontal position to the right of the mobile column 710c and a central position to the right of mobile column 710b (e.g., right of access window 734), and left push arm 742 may be horizontally translated between the leftmost horizontal position to the left of the mobile column 710a and a central position to the right of mobile column 710b (e.g., right of access window 734).

In addition, as shown in FIG. 14H, transfer module 730 may comprise one or more sensors (e.g., sensors 768a, 768b, 768c, collectively referred to herein as sensors 768) configured to detect if a dialysate bag is present in a corresponding space, e.g., positioned in line with shelf 740 in the first loading position (e.g., above the upper end of the respective mobile column, as described above) for automated transfer of the dialysate bag onto shelf 740, e.g., via left push arm 742 or right push arm 744, as well as for automated transfer of the dialysate bag through access window 734 via shelf 740, as described in further detail below. For example, sensors 768 may comprise infrared sensors. Additionally, sensors 768 may be operatively coupled to controller 711 and GUI 701 for transmitting data therebetween for coordinating dispensing of the dialysate bags.

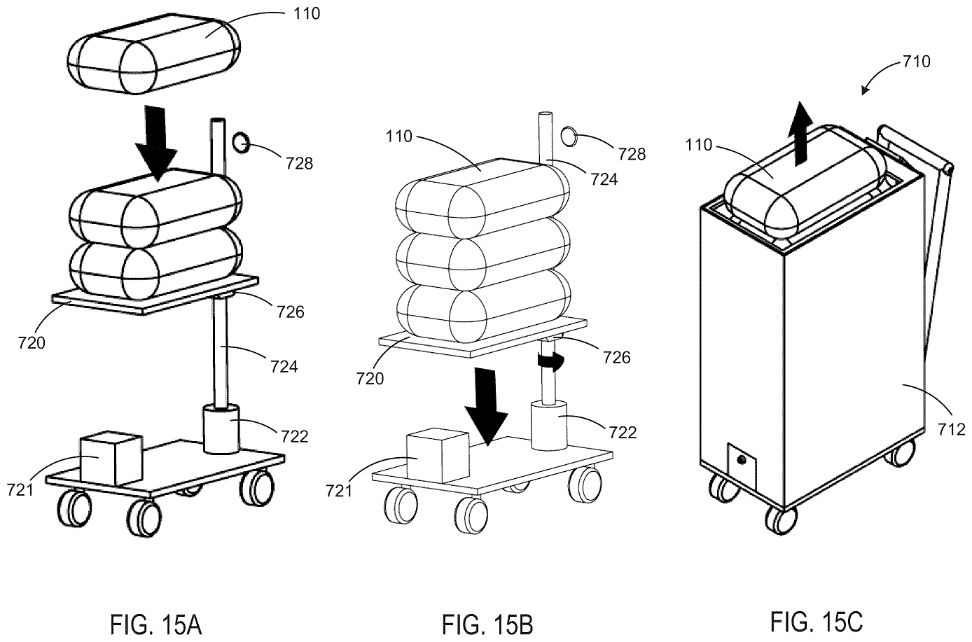

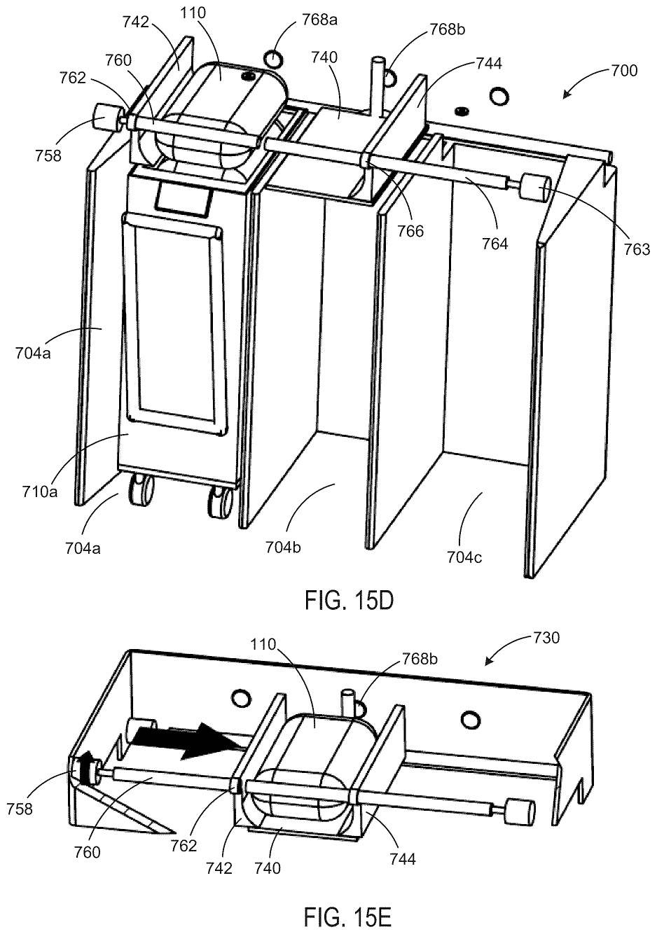

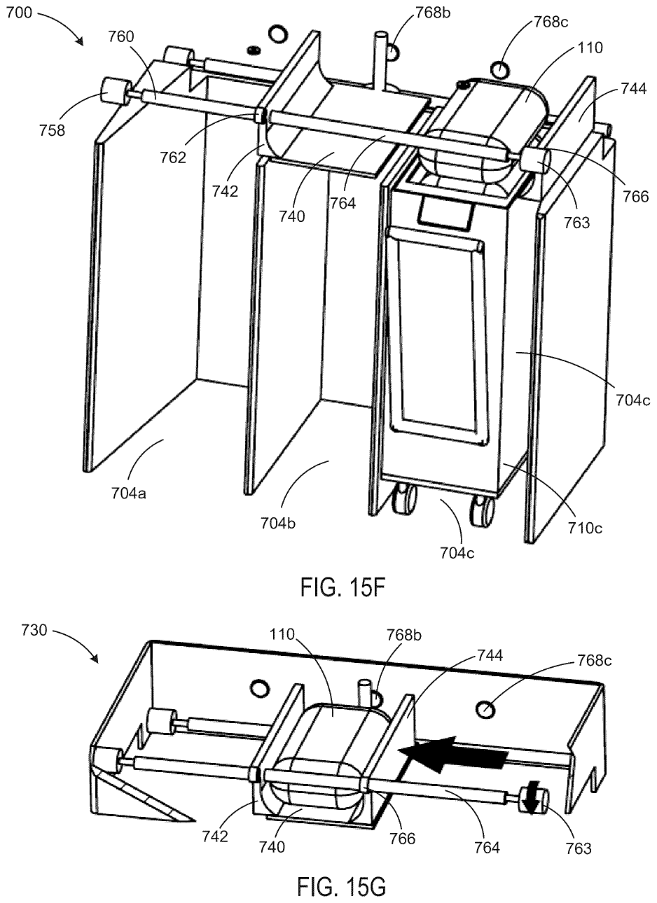

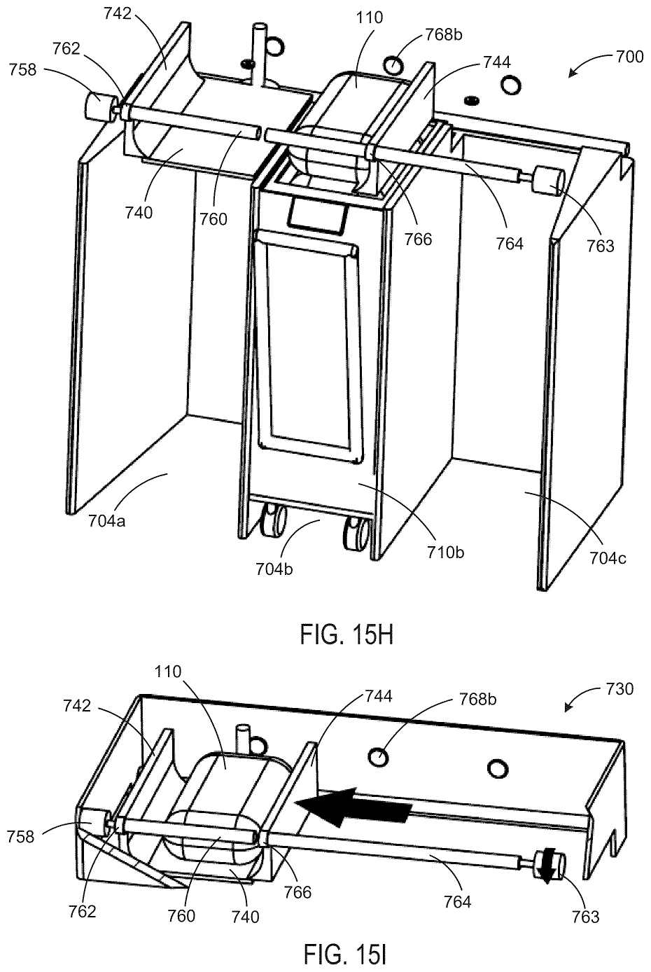

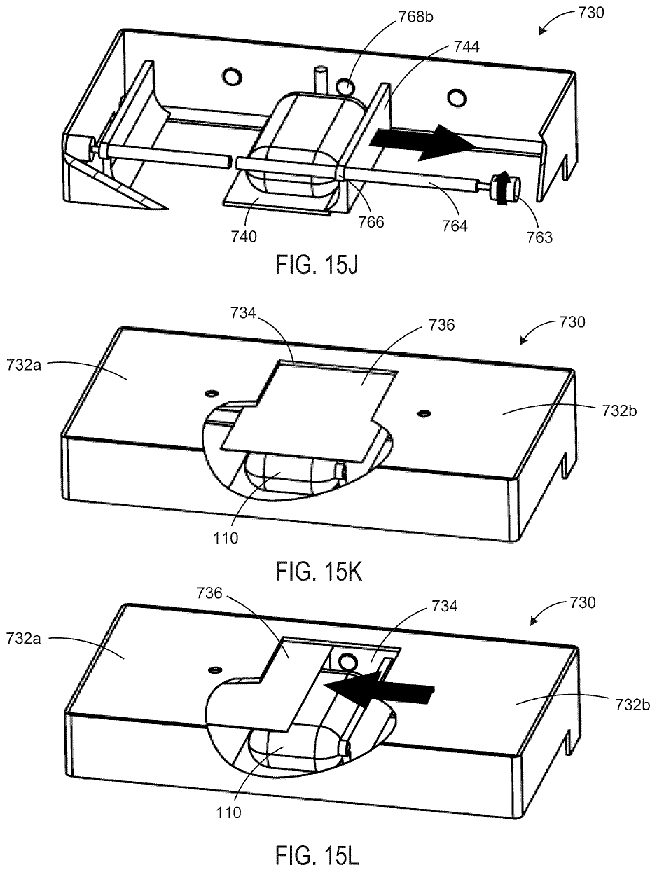

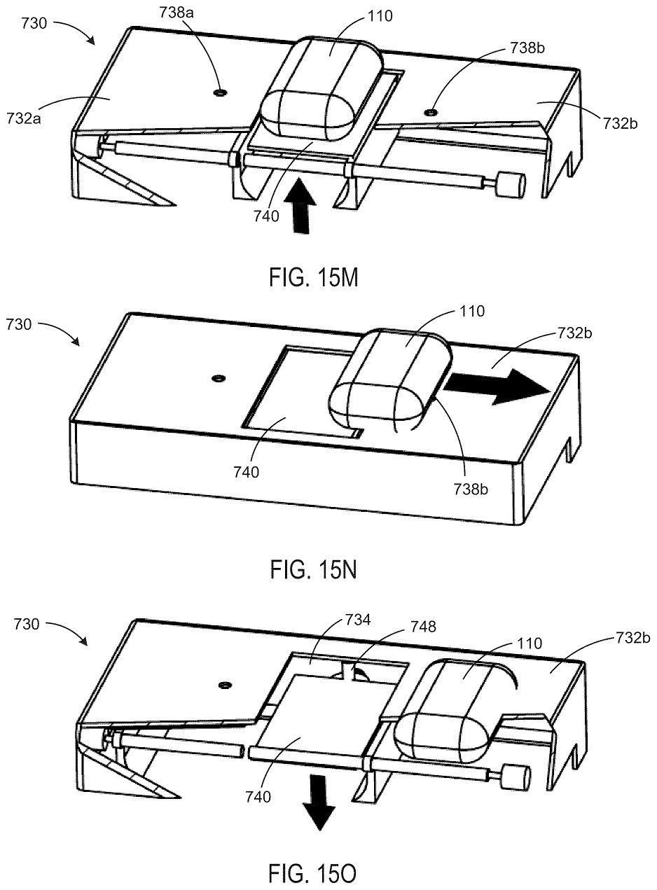

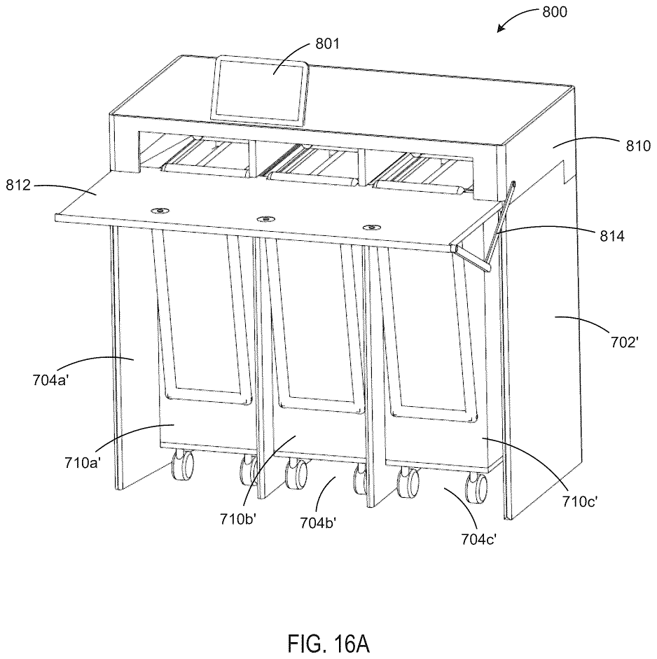

Referring now to FIGS. 15A to 15L, an exemplary method for automated loading and dispensing of dialysate bags via compact automated storage and dispensing system 700 is provided. First, to load one or more dialysate bags 110 into one or more mobile columns 710, the selected mobile column may be wheeled out of its respective docking station, such that the user may manually insert a dialysate bag through the opening at the top of the respective mobile column 710. Prior to loading mobile column 710, shelf 720 may be moved to a vertical position (e.g., via actuation of motor 722 and ball screw shaft 724) such that the top-most slot within mobile column 710 is vacant, and further that either shelf 720, or a dialysate bag disposed thereon, is in the slot immediately beneath the top-most slot, as shown in FIG. 15A. Accordingly, dialysate bag 110 may be placed within the top-most slot of mobile column 710 directly on shelf 720, e.g., if there are no other dialysate bags disposed on shelf 720, or may be placed on top of another dialysate bag that is already placed on shelf 720, as shown in FIG. 15A.

Once dialysate bag 110 is placed in the top-most slot, and detected via sensor 728, sensor 728 may transmit data indicative of the occupation of the top-most slot by dialysate bag 110 to the controller within electronics box 721, which in turn actuates motor 722 to cause rotation of ball screw shaft 724 in the second rotational direction to thereby cause linear movement of nut 726 and, accordingly, shelf 720 along ball screw shaft 724 in the downward vertical direction until sensor 728 no longer detects a dialysate bag in the top-most slot, as shown in FIG. 15B, such that there is enough space to load at least another dialysate bag within the mobile column. This process may be repeated until mobile column 710 is fully loaded with dialysate bags, and similarly may be repeated with the remaining mobile columns. The controller may track the quantity of dialysate bags loaded within the mobile column, such that it may not actuate motor 722 to descend shelf 720 if there is no more space in the mobile column (e.g., if the top-most slot is occupied with a dialysate bag, as detected by sensor 728).

Moreover, as described above, the user may provide user input indicative of the dialysate concentrations of the dialysate bags being loaded into the particular mobile column (e.g., via controller 711), such that the controller may track which dialysate bags having particular dialysate concentrations are stored within which mobile column of system 700. Accordingly, when a dialysate bag from, e.g., mobile column 710a, is desired to be dispensed, the controller of transfer module 730 may actuate motor 758 to cause rotation of ball screw shaft 760 in the second rotational direction to thereby cause linear movement of nut 762 and, accordingly, left push arm 742 along ball screw shaft 760 in the leftward horizontal direction until left push arm 742 is positioned at the leftmost horizontal position (e.g., to the left of mobile column 710a within docking station 704a), as shown in FIG. 15D. Moreover, controller of transfer module 730 simultaneously may actuate motor 763 to cause rotation of ball screw shaft 764 in the first rotational direction to thereby cause linear movement of nut 766 and, accordingly, right push arm 744 along ball screw shaft 764 in the leftward horizontal direction until right push arm 744 is positioned at a central position to the right of shelf 740, which is disposed above docking station 704b in the loading position (e.g., the default low position), as shown in FIG. 15D. Shelf 740 may be positioned in the loading position above docking station 704b and in alignment with access window 734 of transfer module 730 via actuation of motor 746 and vertical ball screw shaft 748 and/or via actuation of motor 752 and horizontal ball screw shaft 754, as described above. As shown in FIG. 15D, in the default loading position, shelf 740 may be aligned with the upper surface of mobile column 710a.

The controller of transfer module 730 may then command the controller of mobile column 710a to actuate motor 722 to cause rotation of ball screw shaft 724 in the first rotational direction to thereby cause linear movement of nut 726 and, accordingly, shelf 720 along ball screw shaft 724 in the upward vertical direction until the top-most dialysate bag 110 is ascended out of body 712 and positioned above the upper surface of the mobile column, as shown in FIG. 15C. Sensor 768a may detect the presence of dialysate bag 110 to confirm that the dialysate bag is properly positioned for loading onto shelf 740. Accordingly, as shown in FIG. 15D, the bottom surface of dialysate bag 110 may be aligned/flushed with shelf 740 in the loading position, and left push arm 742 may be positioned to the left of dialysate bag 110. Next, the controller of transfer module 730 may actuate motor 758 to cause rotation of ball screw shaft 760 in the first rotational direction to thereby cause linear movement of nut 762 and, accordingly, left push arm 742 along ball screw shaft 760 in the rightward horizontal direction until left push arm 742 is positioned at a central position to the left of shelf 740, thereby pushing dialysate bag 110 horizontally rightward over shelf 740, as shown in FIG. 15E. As shown in FIG. 15E, right push arm 744 may prevent dialysate bag 110 from being pushed too far right and falling off of shelf 740, such that dialysate bag 110 is aligned with access window 734 of transfer module 730.

Similarly, when a dialysate bag from, e.g., mobile column 710c, is desired to be dispensed, the controller of transfer module 730 may actuate motor 763 to cause rotation of ball screw shaft 764 in the second rotational direction to thereby cause linear movement of nut 766 and, accordingly, right push arm 744 along ball screw shaft 764 in the rightward horizontal direction until right push arm 744 is positioned at the rightmost horizontal position (e.g., to the right of mobile column 710c within docking station 704c), as shown in FIG. 15F. Moreover, the controller of transfer module 730 simultaneously may actuate motor 758 to cause rotation of ball screw shaft 760 in the first rotational direction to thereby cause linear movement of nut 762 and, accordingly, left push arm 742 along ball screw shaft 760 in the rightward horizontal direction until left push arm 742 is positioned at a central position to the left of shelf 740, which is disposed above docking station 704b in the default loading position, as shown in FIG. 15F. Shelf 740 may be positioned in the loading position above docking station 704b and in alignment with access window 734 of transfer module 730 via actuation of motor 746 and vertical ball screw shaft 748 and/or via actuation of motor 752 and horizontal ball screw shaft 754, as described above. As shown in FIG. 15F, in the default loading position, shelf 740 may be aligned with the upper surface of mobile column 710c.

The controller of transfer module 730 may then command the controller of mobile column 710c to actuate motor 722 to cause rotation of ball screw shaft 724 in the first rotational direction to thereby cause linear movement of nut 726 and, accordingly, shelf 720 along ball screw shaft 724 in the upward vertical direction until the top-most dialysate bag 110 is ascended out of body 712 and positioned above the upper surface of the mobile column, as shown in FIG. 15C. Sensor 768c may detect the presence of dialysate bag 110 to confirm that the dialysate bag is properly positioned for loading onto shelf 740. Accordingly, as shown in FIG. 15F, the bottom surface of dialysate bag 110 may be aligned/flushed with shelf 740 in the loading position, and right push arm 744 may be positioned to the right of dialysate bag 110. Next, the controller of transfer module 730 may actuate motor 763 to cause rotation of ball screw shaft 764 in the first rotational direction to thereby cause linear movement of nut 766 and, accordingly, right push arm 744 along ball screw shaft 764 in the leftward horizontal direction until right push arm 744 is positioned at a central position to the right of shelf 740, thereby pushing dialysate bag 110 horizontally leftward over shelf 740, as shown in FIG. 15G. As shown in FIG. 15G, left push arm 742 may prevent dialysate bag 110 from being pushed too far left and falling off of shelf 740, such that dialysate bag 110 is aligned with access window 734 of transfer module 730.

Moreover, when a dialysate bag from, e.g., mobile column 710b, is desired to be dispensed, the controller of transfer module 730 may actuate motor 758 to cause rotation of ball screw shaft 760 in the second rotational direction to thereby cause linear movement of nut 762 and, accordingly, left push arm 742 along ball screw shaft 760 in the leftward horizontal direction until left push arm 742 is positioned at the leftmost horizontal position (e.g., to the left of mobile column 710a within docking station 704a), as shown in FIG. 15H. Moreover, the controller of transfer module 730 simultaneously may actuate motor 763 to cause rotation of ball screw shaft 764 in the first rotational direction to thereby cause linear movement of nut 766 and, accordingly, right push arm 744 along ball screw shaft 764 in the leftward horizontal direction until right push arm 744 is positioned at a central position to the right of mobile column 710b within docking station 704b, as shown in FIG. 15H. Additionally, the controller of transfer module 730 simultaneously may actuate motor 752 to cause rotation of horizontal ball screw shaft 754 in the second rotational direction to thereby cause linear movement of nut 756 and, accordingly, shelf 740 along ball screw shaft 754 in the leftward horizontal direction until shelf 740 is positioned at the leftmost horizontal position (e.g., above docking station 704a), as shown in FIG. 15H, such that the opening at the top of mobile column 710b is unobstructed. As shown in FIG. 15H, in the default loading position, shelf 740 may be aligned/flushed with the upper surface of mobile column 710b.

The controller of transfer module 730 may then command the controller of mobile column 710b to actuate motor 722 to cause rotation of ball screw shaft 724 in the first rotational direction to thereby cause linear movement of nut 726 and, accordingly, shelf 720 along ball screw shaft 724 in the upward vertical direction until the top-most dialysate bag 110 is ascended out of body 712 and positioned above the upper surface of the mobile column, as shown in FIG. 15C. Sensor 768b may detect the presence of dialysate bag 110 to confirm that the dialysate bag is properly positioned for loading onto shelf 740. Accordingly, as shown in FIG. 15H, the bottom surface of dialysate bag 110 may be aligned/flushed with shelf 740 in the loading position, and right push arm 744 may be positioned to the right of dialysate bag 110. Next, the controller of transfer module 730 may actuate motor 763 to cause rotation of ball screw shaft 764 in the first rotational direction to thereby cause linear movement of nut 766 and, accordingly, right push arm 744 along ball screw shaft 764 in the leftward horizontal direction until right push arm 744 is positioned to the right of shelf 740, thereby pushing dialysate bag 110 horizontally leftward over shelf 740, as shown in FIG. 15I. As shown in FIG. 15I, left push arm 742 may prevent dialysate bag 110 from being pushed too far left and falling off of shelf 740. Sensor 768a may detect the presence of dialysate bag 110 to confirm that the dialysate bag is properly positioned on shelf 740 above docking station 704a.

The controller of transfer module 730 may then actuate motor 763 to cause rotation of ball screw shaft 764 in the second rotational direction to thereby cause linear movement of nut 766 and, accordingly, right push arm 744 along ball screw shaft 766 in the rightward horizontal direction until right push arm 744 is again positioned in the central position to the right of mobile column 710b within docking station 704b, as shown in FIG. 15J. Additionally, the controller of transfer module 730 simultaneously may actuate motor 752 to cause rotation of horizontal ball screw shaft 754 in the first rotational direction to thereby cause linear movement of nut 756 and, accordingly, shelf 740 along ball screw shaft 754 in the rightward horizontal direction until shelf 740 is positioned above mobile column 710b within docking station 704b and in alignment with access window 734. Sensor 768b may detect the presence of dialysate bag 110 to confirm that the dialysate bag is properly positioned on shelf 740 above docking station 704b for advancement through access window 734.

Next, whether the dialysate bag loaded onto shelf 740 was received from mobile column 710a, mobile column 710b, or mobile column 710c, as described above, shelf 740, which is aligned with access window 734 of transfer module 730, as shown in FIG. 15K, may be actuated to dispense dialysate bag 110 disposed thereon through access window 734 of transfer module 730. For example, as shown in FIG. 15L, cover 736 may be actuated to transition from the closed state to the open state, such that access window 734 is unobstructed. The controller of transfer module 730 may then actuate motor 746 to thereby cause rotation of ball screw shaft 748 in the first rotational direction to thereby cause linear movement of nut 750 and, accordingly, shelf 740 along ball screw shaft 748 in the upward vertical direction, such that dialysate bag 110 is ascended through access window 734 via shelf 740, as shown in FIG. 15M. Sensor 768b may detect the absence of dialysate bag 110 to confirm that the dialysate bag is no longer positioned beneath access window 734.

As shown in FIG. 15M, when shelf 740 is in the second dispensing position, shelf 740 may be vertically aligned/flushed with the top surface of transfer module 730, such that the dialysate bag disposed thereon may easily be transferred on left top surface 732a or right top surface 732b, as shown in FIG. 15N. For example, load cell 738b may detect the weight of dialysate bag 110 to confirm that the dialysate bag is properly positioned on right top surface 732b of transfer module, e.g., for automated connection with a cycler solution line via case 200, as described above. Cover 736 may then be actuated to transition from the open state to the closed state to thereby obstruct access window 734. To dispense one or more additional dialysate bags, the controller of transfer module 730 may then actuate motor 746 to thereby cause rotation of ball screw shaft 748 in the second rotational direction to thereby cause linear movement of nut 750 and, accordingly, shelf 740 along ball screw shaft 748 in the downward vertical direction, such that shelf 740 is in the default loading position, as shown in FIG. 15O. Accordingly, the processes described above may be repeated to automatically dispense another dialysate bag received from the desired mobile column through access window 734 for placement on left top surface 732a, the presence of which may be confirmed with load cell 738a, as described above.

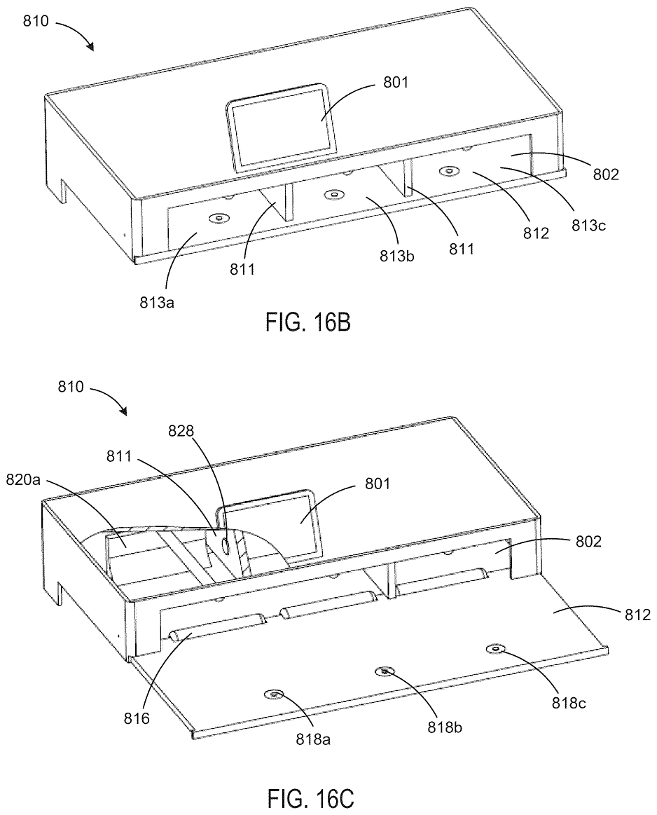

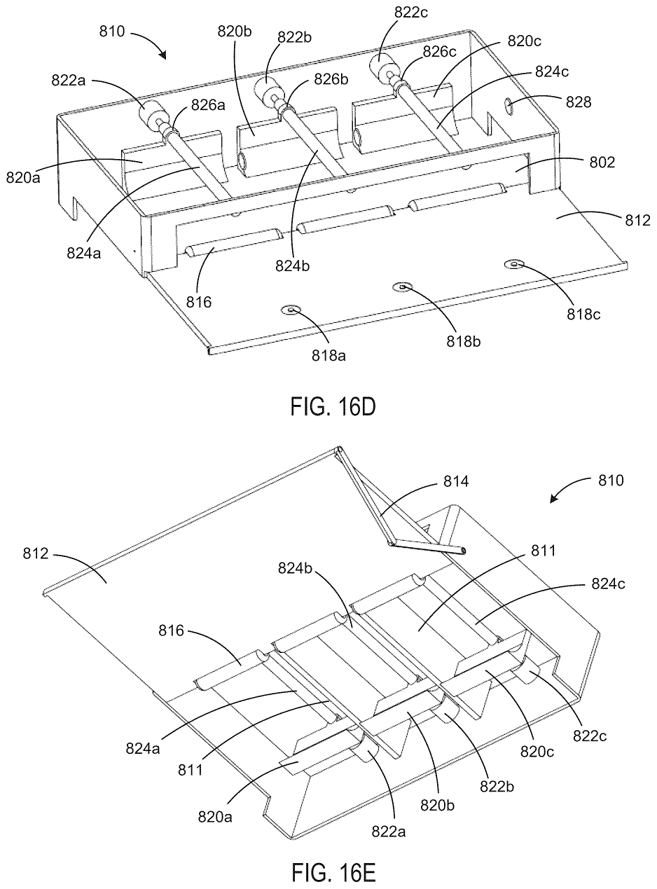

Referring now to FIGS. 16A to 16E, an alternative exemplary compact dispensing system for automated storage and dispensing of dialysate bags used in an automated peritoneal dialysis (APD) treatment is provided. As shown in FIG. 16A, compact automated storage and dispensing system 800 may be constructed similar to compact automated storage and dispensing system 700, with similar components having like-prime reference numerals. For example, plurality of vertical stands 702 defining a plurality of docking stations 704a′, 704b′, 704c′ (collectively referred to herein as docking stations 704′), each sized and shaped to removably receive mobile columns 710a′, 710b′, 710c′ (collectively referred to herein as mobile columns 710′) therein correspond with plurality of vertical stands 702, plurality of docking stations 704a, 704b, 704c, and mobile columns 710a, 710b, 710c. Like system 700, system 800 may be used as a standalone device that may be used with any dialysis treatment system and/or may be integrated with any of the connection cases (e.g., cases 200, 300, 500) and dialysate samplers (e.g., sampler case 600) of automated dialysis line connection system 100, as described above.

System 800 differs from system 700 in that system 800 comprises transfer module 810. As shown in FIGS. 16A and 16B, compact automated storage and dispensing system 800 may include a graphical user interface, e.g., GUI 801, which may be constructed similar to GUI 701. For example, GUI 801 may be configured to be operatively coupled to the controllers of each of mobile columns 710′, for electrical communication therebetween, and may be disposed on or, otherwise, integrated with transfer module 810. As shown in 16B, transfer module 810 may comprise a plurality of dividers 811 (e.g., interior walls) defining a plurality of compartments 813a, 813b, 813c (collectively referred to herein as compartments 813), each sized and shaped to receive a dialysate bag therein, e.g., when dispensed from mobile columns 710′, as described in further detail below. For example, compartments 813a, 813b, 813c may be disposed over docking stations 704a′, 704b′, 704c′, respectively, and accordingly, mobile columns 710a′, 710b′, 710c′ disposed therein.

Moreover, transfer module 810 may comprise retractable transfer shelf 812, which may be configured to transition between a retracted state, as shown in FIG. 16B, and an extended state, as shown in FIG. 16C. For example, transfer shelf 812 may be slidably coupled to transfer module 810 via a plurality of rails (not shown) affixed to transfer module 810, such that transfer shelf 812 may be pulled by the user to transition from the retracted state to the extended state, and pushed by the user to transition from the extended state to the retracted state. Moreover, the front end of the plurality of rails may have corresponding grooves configured to releasably lock transfer shelf 812 in the extended state. In some embodiments, movement of transfer shelf 812 may be driven via a motor (not shown) operatively coupled to GUI 801, such that the user may actuate movement of transfer shelf 812 by providing user input via GUI 801. In the retracted state, transfer shelf 812 may define the bottom surface of transfer module 810, and further may be disposed above the upper surfaces of mobile columns 710′ within docking stations 704′.

In the extended state, transfer shelf 812 may be configured to receive one or more dialysate bags thereon, e.g., when dispensed from mobile columns 710′ via access window 802 of transfer module 810, as described in further detail below. Accordingly, access window 802 may be sized and shaped to permit dialysate bags to be advanced therethrough, e.g., via each of compartments 813a, 813b, 813c. Moreover, when transfer shelf 812 is in the extended state, compartments 813a, 813b, 813c are in communication with mobile columns 710a′, 710b′, 710c′, such that one or more dialysate bags may be ascended into a respective compartment from a respective mobile column, as described above with respect to FIG. 15C. As shown in FIG. 16C, transfer shelf 812 further may include a plurality of rollers 816 disposed on the rear end of transfer shelf 812 and configured to facilitate transfer of the dialysate bags from compartments 813 onto transfer shelf 812. In some embodiments, transfer module may include a pair of folding arms 814, e.g., disposed on the right and left sides of transfer shelf 812, as shown in FIG. 16A. Folding arms 814 may be configured to transition between a folded state, e.g., when transfer shelf 812 is in the retracted state, and an expanded state, e.g., when transfer shelf 812 is in the extended state. In the expanded state, folding arms 814 are configured to support the weight of a plurality of dialysate bags (e.g., three dialysate bags) disposed on transfer shelf 812, as well as to prevent deflection of transfer shelf 812.

As shown FIGS. 16B and 16C, transfer shelf 812 further may include one or more sensors (e.g., load cell 818a horizontally aligned with compartment 813a, load cell 818b horizontally aligned with compartment 813b, and load cell 818a horizontally aligned with compartment 813) each configured to detect a weight thereon to determine whether a dialysate bag is currently disposed at the corresponding location on transfer shelf 812. Moreover, transfer module 810 may include a plurality of sensors 828 (e.g., infrared sensors) disposed within each of compartments 813a, 813b, 813c and configured to detect if a dialysate bag is currently disposed therein. For example, as shown in FIG. 16C, sensor 828 for detecting whether a dialysate bag is stored within compartment 813a may be disposed on divider 811, and as shown in FIG. 16D, sensor 828 for detecting whether a dialysate bag is stored within compartment 813c may be disposed on an inner wall of transfer module 810. Load cells 818a, 818b, 818c and sensors 828 may each be operatively coupled to the controllers of mobile columns 710′ and GUI 801 for transmitting data therebetween for coordinating dispensing of the dialysate bags.

As shown in FIGS. 16D and 16E, transfer module 810 may include push arms 820a, 820b, 820c (collectively referred to herein as push arms 820) slidably disposed within compartments 813a, 813b, 813c, respectively, and configured to translate linearly along the horizontal direction to push a dialysate bag horizontally from the respective compartment onto transfer shelf 812. For example, each push arm 820a, 820b, 820c may be mounted to nuts 826a, 826b, 826c (collectively referred to herein as nuts 826, e.g., threaded nuts), respectively, which may be slidably coupled to horizontal ball screw shafts 824a, 824b, 824c (collectively referred to herein as horizontal ball screw shafts 824) operatively coupled to and extending horizontally from motors 822a, 822b, 822c (collectively referred to herein as motors 822), respectively. Motors 822 and horizontal ball screw shaft 824 may be affixed to a wall of transfer module 810.

Accordingly, motors 822 may be independently actuated (e.g., via the controller of GUI 801) to cause rotation of ball screw shafts 824 in a first rotational direction, thereby causing linear movement of nuts 826 and, accordingly, push arms 820 along ball screw shafts 824 in a first horizontal direction (e.g., forward), and further may be independently actuated to cause rotation of ball screw shafts 824 in a second rotational direction opposite to the first rotational direction, thereby causing linear movement of nuts 826 and, accordingly, push arms 820 along ball screw shafts 824 in a second horizontal direction opposite to the first horizontal direction (e.g., rearward). Horizontal ball screw shafts 824 may each have a length spanning across at least the depth of transfer module 810 (e.g., from the rear end to the front end of transfer module 810), such that push arms 820 may each be horizontally translated between a rearmost horizontal position to the rear of mobile columns 710′ and a frontmost position adjacent to the front of mobile columns 710′ (e.g., adjacent to access window 802).

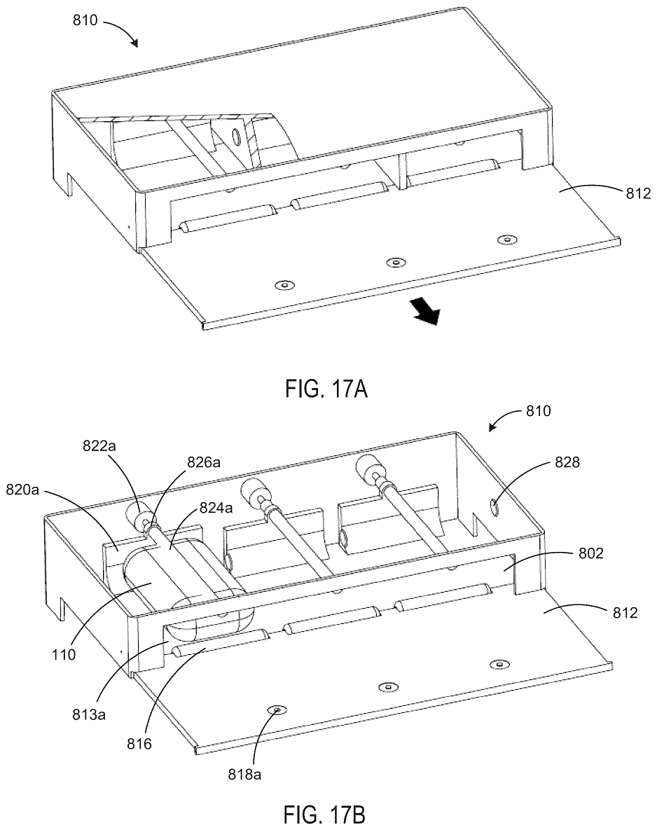

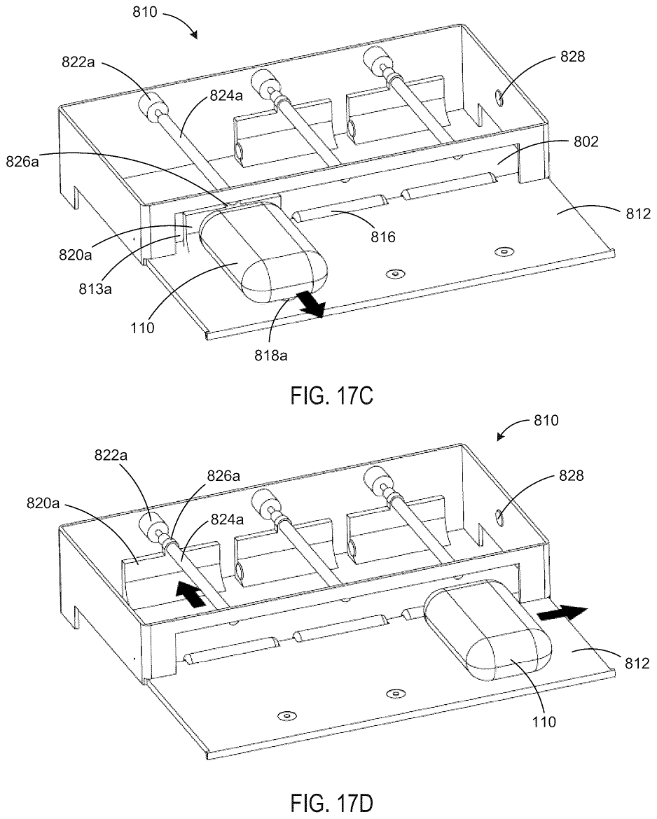

Referring now to FIGS. 17A to 17D, an exemplary method for automated loading and dispensing of dialysate bags via compact automated storage and dispensing system 800 is provided. Mobile columns 710′ may be loaded with dialysate bags and dispense dialysate bags one at a time, as described above with regard to FIGS. 15A to 15C. For example, to load one or more dialysate bags 110 into one or more mobile columns 710′, the selected mobile column may be wheeled out of its respective docking station, such that the user may manually insert a dialysate bag through the opening at the top of the respective mobile column 710′. As described above, GUI 801 may receive and display user input indicative of, e.g., dialysate concentrations of the dialysate bags loaded into the particular mobile column, as well as the quantity of dialysate bags stored within each mobile column.

To begin dispensing one or more dialysate bags from mobile columns 710′, transfer shelf 812 may be transitioned to the extended state, as shown in FIG. 17A. Accordingly, when a dialysate bag from mobile column, e.g., mobile column 710a′, is desired to be dispensed, the controller of transfer module 810 may command the controller of mobile column 710a′ to ascend the top-most dialysate bag 110 to a position above the upper surface of mobile column 710a′, as described above, such that the top-most dialysate bag 110 is disposed within compartment 813a, as shown in FIG. 17B. Sensor 828 in communication with compartment 813a may detect the presence of dialysate bag 110 to confirm that the dialysate bag is properly positioned within compartment 813a for loading onto transfer shelf 812. The controller of transfer module 810 may then actuate motor 822a to cause rotation of ball screw shaft 824a in the first rotational direction to thereby cause linear movement of nut 826a and, accordingly, push arm 820a along ball screw shaft 824a in the forward horizontal direction until push arm 824a is positioned at the frontmost horizontal position adjacent to access window 802 (e.g., to the front of mobile column 710a′), thereby pushing dialysate bag 110 horizontally forward over rollers 816 and onto transfer shelf 812, as shown in FIG. 17C. Sensor 818a may detect the presence of dialysate bag 110 to confirm that the dialysate bag is properly positioned on transfer shelf 812. The user may then manually move dialysate bag to a desired location, as shown in FIG. 17D, e.g., for automated connection to a cycler line, as described above.

Moreover, to dispense one or more additional dialysate bags from mobile column 710a′, the controller of transfer module 810 may then actuate motor 822a to thereby cause rotation of ball screw shaft 824a in the second rotational direction to thereby cause linear movement of nut 826a and, accordingly, push arm 820a along ball screw shaft 824a in the rearward horizontal direction, such that push arm 820a is in the rearmost position, as shown in FIG. 17D. Accordingly, the processes described above may be repeated to automatically dispense another dialysate bag received from any one of mobile columns 710a′, 710b′, 710c′ on transfer shelf 812. If the user requires an additional dialysate bag from the same mobile column (e.g., two bags from mobile column 710a′), and load cell 818a detects the presence of the first dispense dialysate bag thereon, GUI 801 may alert the user (e.g., via a visual and/or audible alert) to manually slide the first dispensed dialysate bag to a vacant space on transfer shelf 812, which is confirmed by the corresponding load cells.

While various illustrative embodiments of the invention are described above, it will be apparent to one skilled in the art that various changes and modifications may be made therein without departing from the invention. The appended claims are intended to cover all such changes and modifications that fall within the true scope of the invention.

What is claimed:

1. A system for automatically connecting a pair of dialysis lines of a dialysis circuit, the system comprising:
a fixed platform comprising a first slot configured to receive a first connector of a first dialysis line;
a movable platform configured to move in at least two degrees of freedom relative to the fixed platform, the movable platform comprising a second slot configured to receive a second connector of a second dialysis line;
a drive gear coupled to the fixed platform or the movable platform at an incline angle relative to the first slot or the second slot, the drive gear configured to be actuated to transmit rotary motion and linear motion to the first connector or the second connector; and
a controller operatively coupled to one or more motors of the movable platform, the controller configured to:
cause the one or more motors to move the movable platform in a first direction along a first degree of freedom to coaxially align the second connector with the first connector;
cause the one or more motors to move the movable platform in a first direction along a second degree of freedom to engage the second connector with the first connector; and
actuate the drive gear to transmit rotary motion and linear motion to the first connector or the second connector to cause the first connector or the second connector to be coupled to the second connector or the first connector, respectively.

2. The system of claim 1, further comprising:
a first slide table comprising a first stationary frame and first slide block configured to move relative to the first stationary frame along the first or second degree of freedom, the first slide block coupled to the movable platform; and
a second slide table comprising a second stationary frame and second slide block configured to move relative to the second stationary frame along the second or first degree of freedom, the second slide block coupled to the first stationary frame,
wherein the second stationary frame is fixed relative to the fixed platform.

3. The system of claim 2, wherein each of the first and second slide tables comprise:
a threaded axel extending longitudinally between first and second ends of the respective first and second stationary frames, the threaded axel operatively coupled the one or more motors of the movable platform; and
one or more support rods extending longitudinally between the first and second ends of the respective first and second stationary frames,
wherein the first and second slide blocks each comprises a threaded channel configured to slidably receive the respective threaded axel therethrough, and wherein rotation of the respective threaded axel via the one or more motors of the movable platform causes the respective first and second slide blocks to slide along the respective threaded axel and one or more support rods.

4. The system of claim 2, wherein the first slide table is offset from the second slide table by an angle of 90 degrees.

5. The system of claim 1, wherein the first connector comprises a plurality of ridges, a lip, and a removable cap comprising a pull ring,
wherein the first slot comprises a cavity configured to receive the lip in a manner that prevents axial movement between the first connector and the first slot, a plurality of grooves configured to receive at least a portion of the plurality of ridges in a manner that prevents rotation between the first connector and the first slot, and a channel configured to be aligned with a tubing of the first dialysis line when the first connector is received within the first slot, and
wherein the movable platform comprises a hook configured to be wrapped by the pull ring of the removable cap of the first connector, the system further comprising:
a clamp block operatively coupled to a clamp motor, the clamp motor configured to be actuated via the controller to move the clamp block through the channel between a first disengaged position where the clamp block is disengaged from the tubing of the first dialysis line, and a second engaged position where the clamp block engages the tubing of the first dialysis line to thereby prevent fluid flow between the first connector and the tubing of the first dialysis line.

6. The system of claim 1, wherein the first connector comprises a twistable lock, an indented retention portion, and a removable cap comprising one or more gripping portions, the system further comprising a retention gear configured to be disposed over the indented retention portion in a manner that permits transmission of rotary motion from the retention gear to the first connector.

7. The system of claim 6, wherein the first slot comprises a cavity configured to slidably receive the retainer gear therein, an opening disposed within the cavity, and a channel configured to be aligned with the twistable lock when the first connector is received within the first slot, and
wherein the movable platform comprises a cap holder configured to releasably receive the one or more gripping portions of the removable cap of the first connector, the system further comprising:
a lock block operatively coupled to a lock motor, the lock motor configured to be actuated via the controller to move the lock block through the channel between a first disengaged position where the lock block is disengaged from the twistable lock, and a second engaged position where the lock block engages the twistable lock to thereby inhibit movement between the twistable lock and the first slot,
wherein the drive gear is coupled to the fixed platform, such that the drive gear extends at least partially through the opening to contact the retainer gear when the first connector is received within the first slot to thereby transmit rotary motion to the first connector.

8. The system of claim 1, wherein the second connector comprises a flat retention portion, a lip, and a removable cap comprising a pull ring, the system further comprising a retention gear configured to be disposed over the flat retention portion in a manner that permits transmission of rotary motion and linear motion from the retention gear to the second connector.

9. The system of claim 8, wherein the second slot comprises a cavity configured to slidably receive the retainer gear therein, an opening disposed within the cavity, and a channel configured to be aligned with a tubing of the second dialysis line when the second connector is received within the second slot,
wherein the fixed platform comprises a hook configured to be wrapped by the pull ring of the removable cap of the second connector, the system further comprising:
a clamp block operatively coupled to a clamp motor, the clamp motor configured to be actuated via the controller to move the clamp block through the channel between a first disengaged position where the clamp block is disengaged from the tubing of the second dialysis line, and a second engaged position where the clamp block engages the tubing of the second dialysis line to thereby prevent fluid flow between the second connector and the tubing of the second dialysis line,
wherein the drive gear is coupled to the movable platform at the incline angle relative to the second slot, the drive gear extending at least partially through the opening to contact the retainer gear when the second connector is received within the second slot to thereby transmit rotary motion and linear motion to the second connector.

10. The system of claim 1, wherein the second connector comprises a plurality of ridges, a lip, and a removable cap comprising a pull ring,
wherein the second slot comprises a cavity comprising plurality of grooves configured to receive at least a portion of the plurality of ridges in a manner that prevents rotation between the second connector and the second slot, and a channel configured to be aligned with a tubing of the second dialysis line when the second connector is received within the second slot, and
wherein the fixed platform comprises a hook configured to be wrapped by the pull ring of the removable cap of the second connector, the system further comprising:
a clamp block operatively coupled to a clamp motor, the clamp motor configured to be actuated via the controller to move the clamp block through the channel between a first disengaged position where the clamp block is disengaged from the tubing of the second dialysis line, and a second engaged position where the clamp block engages the tubing of the second dialysis line to thereby prevent fluid flow between the second connector and the tubing of the second dialysis line.

11. The system of claim 10, wherein the movable platform further comprises a tube holder configured to releasably receive at least a portion of the tubing of the second dialysis line therein.

12. An automated dialysis system comprising:
a first case comprising the system of claim 1, wherein the first dialysis line of the first case comprises a bag line fluidically coupled to a dialysis bag comprising dialysate, and wherein the second dialysis line of the first case comprises a cycler solution line operatively coupled to a cycler;
a second case comprising the system of claim 1, wherein the first dialysis line of the second case comprises a transfer set configured to be fluidically coupled to a patient's body, and wherein the second dialysis line of the second case comprises a cycler patient line operatively coupled to the cycler; and
a third case comprising the system of claim 1, wherein the first dialysis line of the third case comprises a drain bag line fluidically coupled to a drain bag, and wherein the second dialysis line of the third case comprises a second cycler drain line operatively coupled to the cycler.

13. The system of claim 12, wherein the controller is configured to:
cause the first case to fluidically couple the bag line with the cycler solution line and cause the third case to fluidically couple the drain bag line with the cycler drain line;
cause the cycler to complete a priming process; and
cause, upon completion of the priming process, the second case to fluidically couple the transfer set with the cycler solution line.

14. The system of claim 13, wherein the controller is configured to receive a signal from the cycler indicative of the completion of the priming process.

15. The system of claim 13, further comprising:
a sensor configured to measure a fluid level within the cycler patient line,
wherein the controller is configured to determine that the priming process is complete based on the fluid level within the cycler patient line.

16. The system of claim 12, further comprising a sensor operatively coupled to the third case and configured to detect a velocity of fluid flow through the drain bag line.

17. The system of claim 16, wherein the controller is configured to cause the cycler to cease draining of fluid from the patient's body when the velocity of fluid flow through the drain bag line falls below a predetermined threshold.

18. The system of claim 16, wherein the controller is configured to:
calculate an amount of fluid drained from the patient's body at the end of a first infusion cycle based at least partially on the velocity of fluid flow through the drain bag line;
estimate an amount of fluid remaining within the patient's body at the end of the first infusion cycle based on the amount of fluid drained from the patient's body at the end of a first infusion cycle;
determine an adjusted amount of fluid to be infused into the patient's body via the transfer set based on the estimated amount of fluid remaining within the patient's body at the end of the first infusion cycle; and
cause, during a subsequent infusion cycle, the cycler to infuse the adjusted amount of fluid into the patient's body via the transfer set to thereby avoid overfilling the patient's body.

19. The system of claim 12, further comprising:
a sensor configured to measure at least one of a glucose, blood urea nitrogen (BUN), or creatinine concentration of fluid exiting the patient's body,
wherein the controller is configured to cause the cycler to initiate drainage of fluid from the patient's body via the drain bag line when the at least one of the glucose, BUN, or creatinine concentration of the fluid exiting the patient's body is within a predetermined range.

20. The system of claim 12, further comprising an automated clamping system for clamping one or more unused dialysis lines, the automated clamping system comprising:
a third slot comprising a cavity configured to receive at least a portion of a third connector of a third dialysis line, and a channel configured to be aligned with a tubing of the third dialysis line when the third connector is received within the third slot; and
a clamp block operatively coupled to a clamp motor, the clamp motor configured to be actuated via the controller to move the clamp block through the channel between a first disengaged position where the clamp block is disengaged from the tubing of the third dialysis line, and a second engaged position where the clamp block engages the tubing of the third dialysis line to thereby prevent fluid flow between the third connector and the tubing of the third dialysis line.

21. The system of claim 1, further comprising:
a first cover configured to be removably disposed over the first connector within the first slot; and
a second cover configured to be removably disposed over the second connector within the second slot.
